SEQUENCE LISTING (53 sequences)

SEQ ID NO: 1 - DNA, 810 bp, Artificial Sequence
  pCold2.3scFv (non-blocking)
atgaatcacaaagtgcatcatcatcatcatcatatcgaaggtaggcatatggagctcggtaccctcgagggatcccagatccagctggtgctgagcggccccgaactgaagaaacccggcgagagcgtcaagatctcttgtaaggccagcggctacaccttcaccacctacggcatgtcttgggtgaagcaagcccccggcaagggtttaaagtggatgggctggatcaacaccagctccggcgtgcctacatacgccgacgactttaagggtcgtttcgccttctctttagagacctccgcctccaccgcctatttacagatcaacaatttaaagaacgaggacaccgccacctacttttgcgctcgtggcggaggctactacggagcctactacttctactattggggccaaggtacaacactgaccgtgtcttctggtggcggcggcagcggcggtggcggctctggcggtggtggcagcgatattcagatgacccagtcccccgcttctttaagcgctagcgtgggagagaccgtgaccatcacttgtagaaccagcgataacatcaacagctatttagcttggtatttacagaggcaaggtaagagcccccagctgctcgtgtacaacgccaagactttaaccgagggcgtgccttctcgtttcagcggcagcggaagcggcacccagttctctttaaagattaacagcctccagcccgaggacttcggcagctactactgccagcaccactacggccctccccccacatttggcggcggcacaaagctcgaaatcaagtaatag

SEQ ID NO: 2 - protein, 268 aa, Artificial Sequence
  pCold2.3scFv (non-blocking) protein
MNHKVHHHHHHIEGRHMELGTLEGSQIQLVLSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSGGGGSGGGGSGGGGSDIQMTQSPASLSASVGETVTITCRTSDNINSYLAWYLQRQGKSPQLLVYNAKTLTEGVPSRFSGSGSGTQFSLKINSLQPEDFGSYYCQHHYGPPPTFGGGTKLEIK

SEQ ID NO: 3 - DNA, 1809 bp, Artificial Sequence
  pEE12.4/anti-Pselectin 2.3scFv-Crry (non- blocking)
atgtccgtgcccacccaagtgctgggtttattattactgtggctgaccgatgccagatgccagatccagctggtgctgagcggccccgaactgaagaaacccggcgagagcgtcaagatctcttgtaaggccagcggctacaccttcaccacctacggcatgtcttgggtgaagcaagcccccggcaagggtttaaagtggatgggctggatcaacaccagctccggcgtgcctacatacgccgacgactttaagggtcgtttcgccttctctttagagacctccgcctccaccgcctatttacagatcaacaatttaaagaacgaggacaccgccacctacttttgcgctcgtggcggaggctactacggagcctactacttctactattggggccaaggtacaacactgaccgtgtcttctggtggcggcggcagcggcggtggcggctctggcggtggtggcagcgatattcagatgacccagtcccccgcttctttaagcgctagcgtgggagagaccgtgaccatcacttgtagaaccagcgataacatcaacagctatttagcttggtatttacagaggcaaggtaagagcccccagctgctcgtgtacaacgccaagactttaaccgagggcgtgccttctcgtttcagcggcagcggaagcggcacccagttctctttaaagattaacagcctccagcccgaggacttcggcagctactactgccagcaccactacggccctccccccacatttggcggcggcacaaagctcgaaatcaagggcggaggtgggtcgggtggcggcggatcttgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattcagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatcatcgagggcaggcatcaccaccatcaccactga

SEQ ID NO: 4 - protein, 602 aa, Artificial Sequence
  pEE12.4/anti-Pselectin 2.3scFv-Crry (non- blocking) protein
MSVPTQVLGLLLLWLTDARCQIQLVLSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSGGGGSGGGGSGGGGSDIQMTQSPASLSASVGETVTITCRTSDNINSYLAWYLQRQGKSPQLLVYNAKTLTEGVPSRFSGSGSGTQFSLKINSLQPEDFGSYYCQHHYGPPPTFGGGTKLEIKGGGGSGGGGSCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSIIEGRHHHHHH

SEQ ID NO: 5 - DNA, 873 bp, Artificial Sequence
  Pselectin 2.7scFv (non-blocking)
atgcaccatcatcatcaccacatcgaaggcaggtggatatctgcagaattcgcccttcagatccagttgctgcagtctggacctgagctgaagaagcctggagagtcagtcaagatctcctgcaaggcttctgggtataccttcacaacctatggaatgagctgggtgaaacaggctccaggaaagggtttaaagtggatgggctggataaacacctcctctggagtgccaacatatgctgatgacttcaagggacggtttgccttctctttggaaacctctgccagcactgcctatttgcagatcaacaacctcaaaaatgaggacacggctacatatttctgtgcaagaggggggggctactatggtgcctactacttttactactggggccaaggcaccactctcacagtctcctcaaagggcgaattccagcacactggcggccgttactcaggaggcggtggcggctcgggtggcggcggctctacagacacactcctgctatgggtactgctgctctgggttccaggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcatga

SEQ ID NO: 6 - protein, 288 aa, Artificial Sequence
  Pselectin 2.7scFv (non-blocking) Protein
HHHHHHIEGRWISAEFALQIQLLQSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSKGEFQHTGGRYSGGGGGSGGGGSTDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 7 - DNA, 1929 bp, Artificial Sequence
  PEE12.4/Pselectin 2.7scFv-Crry (non-blocking)
atgcccatggggtctctgcaaccgctggccaccttgtacctgctggggatgctggtcgcttccgtgctagcgtggatatctgcagaattcgcccttcagatccagttgctgcagtctggacctgagctgaagaagcctggagagtcagtcaagatctcctgcaaggcttctgggtataccttcacaacctatggaatgagctgggtgaaacaggctccaggaaagggtttaaagtggatgggctggataaacacctcctctggagtgccaacatatgctgatgacttcaagggacggtttgccttctctttggaaacctctgccagcactgcctatttgcagatcaacaacctcaaaaatgaggacacggctacatatttctgtgcaagaggggggggctactatggtgcctactacttttactactggggccaaggcaccactctcacagtctcctcaaagggcgaattccagcacactggcggccgttactcaggaggcggtggcggctcgggtggcggcggctctacagacacactcctgctatgggtactgctgctctgggttccaggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaggtggtggcggttcaggcggaggtggctcttgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattcagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatcatcgagggcaggcatcaccaccatcaccactga

SEQ ID NO: 8 - protein, 642 aa, Artificial Sequence
  PEE12.4/Pselectin 2.7scFv-Crry (non-blocking) protein
MPMGSLQPLATLYLLGMLVASVLAWISAEFALQIQLLQSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSKGEFQHTGGRYSGGGGGSGGGGSTDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSGGGGSGGGGSCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSIIEGRHHHHHH

SEQ ID NO: 9 - DNA, 792 bp, Artificial Sequence
  pCold2.12scFv (blocking)
atgaatcacaaagtgcatcatcatcatcatcatatcgaaggtaggcatatggagctcggtaccctcgagggatccgaggtgaagctggtggagagcggcggaggactggtccaacctggctccagcatgaagctctcctgcaccacctccggcttcaccttctccgactactacatggcctgggtcaggcaggtccctgagaagggcctcgagtgggtggccaacatcaattacgacggcagcagcgcctactacctggactccttcaagagcaggttcaccatcagcagggacaacgagaagaacatcctctacctgcagatgtccagcctcaagagcgaggacaccgccacctattactgcgccaggggcgactggttcgtctactggggccagggcacactcgtcacagtcagcgctggaggaggaggaagcggaggaggaggctccggaggcggaggcagcgatatccagatgacccagagcccctccagcatgtccgcttccctgggagagagagtgaccatcacctgcaaggcctcccaggacatcaacagctacctgaactggttccagcagaagcccggcaagagccccaagaccctcatcttcagggccaacaggctcgtcgacggagtcccttccagattcagcggaagcggcagcggccaagactatagcctcaccatctccagcctggagttcgaagacgtcggcatctactactgcctccagtatgccgagttccccttcacctttggcagcggcaccaagctggagatcaagtgatag

SEQ ID NO: 10 - protein, 262 aa, Artificial Sequence
  pCold2.12scFv (blocking) Protein
MNHKVHHHHHHIEGRHMELGTLEGSEVKLVESGGGLVQPGSSMKLSCTTSGFTFSDYYMAWVRQVPEKGLEWVANINYDGSSAYYLDSFKSRFTISRDNEKNILYLQMSSLKSEDTATYYCARGDWFVYWGQGTLVTVSAGGGGSGGGGSGGGGSDIQMTQSPSSMSASLGERVTITCKASQDINSYLNWFQQKPGKSPKTLIFRANRLVDGVPSRFSGSGSGQDYSLTISSLEFEDVGIYYCLQYAEFPFTFGSGTKLEIK

SEQ ID NO: 11 - DNA, 1833 bp, Artificial Sequence
  pEE12.4/anti-pselectin scFv2.12-Crry (blocking)
atgcctatgggcagcctgcagcccctggctaccctgtacctgctgggaatgctggtcgcctccgtcctggcccaccatcatcatcaccacatcgaaggcagggaggtgaagctggtggagagcggcggaggactggtccaacctggctccagcatgaagctctcctgcaccacctccggcttcaccttctccgactactacatggcctgggtcaggcaggtccctgagaagggcctcgagtgggtggccaacatcaattacgacggcagcagcgcctactacctggactccttcaagagcaggttcaccatcagcagggacaacgagaagaacatcctctacctgcagatgtccagcctcaagagcgaggacaccgccacctattactgcgccaggggcgactggttcgtctactggggccagggcacactcgtcacagtcagcgctggaggaggaggaagcggaggaggaggctccggaggcggaggcagcgatatccagatgacccagagcccctccagcatgtccgcttccctgggagagagagtgaccatcacctgcaaggcctcccaggacatcaacagctacctgaactggttccagcagaagcccggcaagagccccaagaccctcatcttcagggccaacaggctcgtcgacggagtcccttccagattcagcggaagcggcagcggccaagactatagcctcaccatctccagcctggagttcgaagacgtcggcatctactactgcctccagtatgccgagttccccttcacctttggcagcggcaccaagctggagatcaagggcggaggtgggtcgggtggcggcggatcttgcccagccccatcacagcttccttctgccaaacctataaatctaactgatgaatccatgtttcccattggaacatatttgttgtatgaatgtctcccaggatatatcaagaggcagttctctatcacctgcaaacaagactcaacctggacgagtgctgaagataagtgtatacgaaaacaatgtaaaactccttcagatcctgagaatggcttggtacatgtacacacaggcattcagtttggatcccgtattaattatacttgtaatcaaggataccgcctcattggttcctcctctgctgtatgtgtcatcactgatcaaagtgttgattgggatactgaggcacctatttgtgagtggattccttgtgagatacccccaggcattcccaatggagatttcttcagttcaaccagagaagactttcattatggaatggtggttacctaccgctgcaacactgatgcgagagggaaggcgctctttaacctggtgggtgagccctccttatactgtaccagcaacgatggtgaaattggagtctggagcggccctcctcctcagtgcattgaactcaacaaatgtactcctcctccctatgttgaaaatgcagtcatgctgtctgagaacagaagcttgttttccttaagggatattgtggagtttagatgtcaccctggctttatcatgaaaggagccagcagtgtgcattgtcagtccctaaacaaatgggagccagagttaccaagctgcttcaagggagtgatatgtcgtctccctcaggagatgagtggattccagaaggggttgggaatgaaaaaagaatattattatggagagaatgtaaccttggaatgtgaggatgggtatactctagaaggcagttctcaaagccagtgccagtctgatggcagctggaatcctcttctggccaaatgtgtatctcgctcaatcatcgagggcaggcatcaccaccatcaccactga

SEQ ID NO: 12 - protein, 610 aa, Artificial Sequence
  pEE12.4/anti-pselectin scFv2.12-Crry (blocking) Protein
MPMGSLQPLATLYLLGMLVASVLAHHHHHHIEGREVKLVESGGGLVQPGSSMKLSCTTSGFTFSDYYMAWVRQVPEKGLEWVANINYDGSSAYYLDSFKSRFTISRDNEKNILYLQMSSLKSEDTATYYCARGDWFVYWGQGTLVTVSAGGGGSGGGGSGGGGSDIQMTQSPSSMSASLGERVTITCKASQDINSYLNWFQQKPGKSPKTLIFRANRLVDGVPSRFSGSGSGQDYSLTISSLEFEDVGIYYCLQYAEFPFTFGSGTKLEIKGGGGSGGGGSCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSIIEGRHHHHHH

SEQ ID NO: 13 - protein, 8 aa, Artificial Sequence
  2.3scFv HC CDR1 AA
GYTFTTYG

SEQ ID NO: 14 - DNA, 24 bp, Artificial Sequence
  2.3scFv HC CDR1 NA
ggctacaccttcaccacctacggc

SEQ ID NO: 15 - protein, 8 aa, Artificial Sequence
  2.3scFv HC CDR2 AA
INTSSGVP

SEQ ID NO: 16 - DNA, 24 bp, Artificial Sequence
  2.3scFv HC CDR2 NA
atcaacaccagctccggcgtgcct

SEQ ID NO: 17 - protein, 14 aa, Artificial Sequence
  2.3scFv HC CDR3 AA
ARGGGYYGAYYFYY

SEQ ID NO: 18 - DNA, 42 bp, Artificial Sequence
  2.3scFv HC CDR3 NA
gctcgtggcggaggctactacggagcctactacttctactat

SEQ ID NO: 19 - protein, 6 aa, Artificial Sequence
  2.3scFv LC CDR1 AA
DNINSY

SEQ ID NO: 20 - DNA, 18 bp, Artificial Sequence
  2.3scFv LC CDR1 NA
gataacatcaacagctat

SEQ ID NO: 21 - protein, 3 aa, Artificial Sequence
  2.3scFv LC CDR2 AA
NAK

SEQ ID NO: 22 - DNA, 9 bp, Artificial Sequence
  2.3scFv LC CDR2 NA
aacgccaag

SEQ ID NO: 23 - protein, 9 aa, Artificial Sequence
  2.3scFv LC CDR3 AA
QHHYGPPPT

SEQ ID NO: 24 - DNA, 27 bp, Artificial Sequence
  2.3scFv LC CDR3 NA
cagcaccactacggccctccccccaca

SEQ ID NO: 25 - DNA, 24 bp, Artificial Sequence
  2.7scFv HC CDR1 NA
gggtataccttcacaacctatgga

SEQ ID NO: 26 - DNA, 24 bp, Artificial Sequence
  2.7scFv HC CDR2 NA
ataaacacctcctctggagtgcca

SEQ ID NO: 27 - DNA, 42 bp, Artificial Sequence
  2.7scFv HC CDR3 NA
gcaagaggggggggctactatggtgcctactacttttactac

SEQ ID NO: 28 - protein, 10 aa, Artificial Sequence
  2.7scFv LC CDR1 AA
KSVSTSGYSY

SEQ ID NO: 29 - DNA, 30 bp, Artificial Sequence
  2.7scFv LC CDR1 NA
aaaagtgtcagtacatctggctatagttat

SEQ ID NO: 30 - protein, 3 aa, Artificial Sequence
  2.7scFv LC CDR2 AA
LVS

SEQ ID NO: 31 - DNA, 9 bp, Artificial Sequence
  2.7scFv LC CDR2 NA
cttgtatcc

SEQ ID NO: 32 - protein, 16 aa, Artificial Sequence
  2.7scFv LC CDR3 AA
QHIRELTRSEGGPSWK

SEQ ID NO: 33 - DNA, 48 bp, Artificial Sequence
  2.7scFv LC CDR3 NA
cagcacattagggagcttacacgttcggaggggggaccaagctggaaa

SEQ ID NO: 34 - protein, 8 aa, Artificial Sequence
  2.12scFv HC CDR1 AA
GFTFSDYY

SEQ ID NO: 35 - DNA, 24 bp, Artificial Sequence
  2.12scFv HC CDR1 NA
ggcttcaccttctccgactactac

SEQ ID NO: 36 - protein, 8 aa, Artificial Sequence
  2.12scFv HC CDR2 AA
INYDGSSA

SEQ ID NO: 37 - DNA, 24 bp, Artificial Sequence
  2.12scFv HC CDR2 NA
atcaattacgacggcagcagcgcc

SEQ ID NO: 38 - protein, 8 aa, Artificial Sequence
  2.12scFv HC CDR3 AA
ARGDWFVY

SEQ ID NO: 39 - DNA, 24 bp, Artificial Sequence
  2.12scFv HC CDR3 NA
gccaggggcgactggttcgtctac

SEQ ID NO: 40 - protein, 6 aa, Artificial Sequence
  2.12scFv LC CDR1 AA
QDINSY

SEQ ID NO: 41 - DNA, 18 bp, Artificial Sequence
  2.12scFv LC CDR1 NA
caggacatcaacagctac

SEQ ID NO: 42 - protein, 3 aa, Artificial Sequence
  2.12scFv LC CDR2 AA
RAN

SEQ ID NO: 43 - DNA, 9 bp, Artificial Sequence
  2.12scFv LC CDR2 NA
agggccaac

SEQ ID NO: 44 - protein, 9 aa, Artificial Sequence
  2.12scFv LC CDR3 AA
LQYAEFPFT

SEQ ID NO: 45 - DNA, 27 bp, Artificial Sequence
  2.12scFv LC CDR3 NA
ctccagtatgccgagttccccttcacc

SEQ ID NO: 46 - DNA, 2805 bp, Artificial Sequence
  Pselectin2.12scFv-CR1(1-10) DNA
atgcctatgggcagcctgcagcccctggctaccctgtacctgctgggaatgctggtcgcctccgtcctggcccaccatcatcatcaccacatcgaaggcagggaggtgaagctggtggagagcggcggaggactggtccaacctggctccagcatgaagctctcctgcaccacctccggcttcaccttctccgactactacatggcctgggtcaggcaggtccctgagaagggcctcgagtgggtggccaacatcaattacgacggcagcagcgcctactacctggactccttcaagagcaggttcaccatcagcagggacaacgagaagaacatcctctacctgcagatgtccagcctcaagagcgaggacaccgccacctattactgcgccaggggcgactggttcgtctactggggccagggcacactcgtcacagtcagcgctggaggaggaggaagcggaggaggaggctccggaggcggaggcagcgatatccagatgacccagagcccctccagcatgtccgcttccctgggagagagagtgaccatcacctgcaaggcctcccaggacatcaacagctacctgaactggttccagcagaagcccggcaagagccccaagaccctcatcttcagggccaacaggctcgtcgacggagtcccttccagattcagcggaagcggcagcggccaagactatagcctcaccatctccagcctggagttcgaagacgtcggcatctactactgcctccagtatgccgagttccccttcacctttggcagcggcaccaagctggagatcaagggcggaggtgggtcgggtggcggcggatctcagtgcaacgcgccggaatggctgccgtttgcgcgcccgaccaacctgaccgatgaatttgaatttccgattggcacctatctgaactatgaatgccgcccgggctatagcggccgcccgtttagcattatttgcctgaaaaacagcgtgtggaccggcgcgaaagatcgctgccgccgcaaaagctgccgcaacccgccggatccggtgaacggcatggtgcatgtgattaaaggcattcagtttggcagccagattaaatatagctgcaccaaaggctatcgcctgattggcagcagcagcgcgacctgcattattagcggcgataccgtgatttgggataacgaaaccccgatttgcgatcgcattccgtgcggcctgccgccgaccattaccaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaaaccgtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacgcggcgcattggagcaccaaaccgccgatttgccagcgcattccgtgcggcctgccgccgaccattgcgaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattatcgagggcaggcatcaccaccatcaccactga

SEQ ID NO: 47 - protein, 934 aa, Artificial Sequence
  pselectin2.12scFv-CR1(1-10) protein
MPMGSLQPLATLYLLGMLVASVLAHHHHHHIEGREVKLVESGGGLVQPGSSMKLSCTTSGFTFSDYYMAWVRQVPEKGLEWVANINYDGSSAYYLDSFKSRFTISRDNEKNILYLQMSSLKSEDTATYYCARGDWFVYWGQGTLVTVSAGGGGSGGGGSGGGGSDIQMTQSPSSMSASLGERVTITCKASQDINSYLNWFQQKPGKSPKTLIFRANRLVDGVPSRFSGSGSGQDYSLTISSLEFEDVGIYYCLQYAEFPFTFGSGTKLEIKGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIIEGRHHHHHH

SEQ ID NO: 48 - DNA, 4155 bp, Artificial Sequence
  pselectin2.12scFv-CR1(1-17) DNA
atgcctatgggcagcctgcagcccctggctaccctgtacctgctgggaatgctggtcgcctccgtcctggcccaccatcatcatcaccacatcgaaggcagggaggtgaagctggtggagagcggcggaggactggtccaacctggctccagcatgaagctctcctgcaccacctccggcttcaccttctccgactactacatggcctgggtcaggcaggtccctgagaagggcctcgagtgggtggccaacatcaattacgacggcagcagcgcctactacctggactccttcaagagcaggttcaccatcagcagggacaacgagaagaacatcctctacctgcagatgtccagcctcaagagcgaggacaccgccacctattactgcgccaggggcgactggttcgtctactggggccagggcacactcgtcacagtcagcgctggaggaggaggaagcggaggaggaggctccggaggcggaggcagcgatatccagatgacccagagcccctccagcatgtccgcttccctgggagagagagtgaccatcacctgcaaggcctcccaggacatcaacagctacctgaactggttccagcagaagcccggcaagagccccaagaccctcatcttcagggccaacaggctcgtcgacggagtcccttccagattcagcggaagcggcagcggccaagactatagcctcaccatctccagcctggagttcgaagacgtcggcatctactactgcctccagtatgccgagttccccttcacctttggcagcggcaccaagctggagatcaagggcggaggtgggtcgggtggcggcggatctcagtgcaacgcgccggaatggctgccgtttgcgcgcccgaccaacctgaccgatgaatttgaatttccgattggcacctatctgaactatgaatgccgcccgggctatagcggccgcccgtttagcattatttgcctgaaaaacagcgtgtggaccggcgcgaaagatcgctgccgccgcaaaagctgccgcaacccgccggatccggtgaacggcatggtgcatgtgattaaaggcattcagtttggcagccagattaaatatagctgcaccaaaggctatcgcctgattggcagcagcagcgcgacctgcattattagcggcgataccgtgatttgggataacgaaaccccgatttgcgatcgcattccgtgcggcctgccgccgaccattaccaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaaaccgtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacgcggcgcattggagcaccaaaccgccgatttgccagcgcattccgtgcggcctgccgccgaccattgcgaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaagcggtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacaccgcgcattggagcaccaaaccgccgatttgccagcgcattccgtgcggcctgccgccgaccattgcgaacggcgattttattagcaccaaccgcgaaaactttcattatggcagcgtggtgacctatcgctgcaacctgggcagccgcggccgcaaagtgtttgaactggtgggcgaaccgagcatttattgcaccagcaacgatgatcaggtgggcatttggagcggcccggcgccgcagtgcattattatcgagggcaggcatcaccaccatcaccactga

SEQ ID NO: 49 - protein, 1384 aa, Artificial Sequence
  pselectin2.12scFv-CR1(1-17) Protein
MPMGSLQPLATLYLLGMLVASVLAHHHHHHIEGREVKLVESGGGLVQPGSSMKLSCTTSGFTFSDYYMAWVRQVPEKGLEWVANINYDGSSAYYLDSFKSRFTISRDNEKNILYLQMSSLKSEDTATYYCARGDWFVYWGQGTLVTVSAGGGGSGGGGSGGGGSDIQMTQSPSSMSASLGERVTITCKASQDINSYLNWFQQKPGKSPKTLIFRANRLVDGVPSRFSGSGSGQDYSLTISSLEFEDVGIYYCLQYAEFPFTFGSGTKLEIKGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIIEGRHHHHHH

SEQ ID NO: 50 - DNA, 2781 bp, Artificial Sequence
  Pselectin2.3scFv-CR1(1-10) DNA
atgtccgtgcccacccaagtgctgggtttattattactgtggctgaccgatgccagatgccagatccagctggtgctgagcggccccgaactgaagaaacccggcgagagcgtcaagatctcttgtaaggccagcggctacaccttcaccacctacggcatgtcttgggtgaagcaagcccccggcaagggtttaaagtggatgggctggatcaacaccagctccggcgtgcctacatacgccgacgactttaagggtcgtttcgccttctctttagagacctccgcctccaccgcctatttacagatcaacaatttaaagaacgaggacaccgccacctacttttgcgctcgtggcggaggctactacggagcctactacttctactattggggccaaggtacaacactgaccgtgtcttctggtggcggcggcagcggcggtggcggctctggcggtggtggcagcgatattcagatgacccagtcccccgcttctttaagcgctagcgtgggagagaccgtgaccatcacttgtagaaccagcgataacatcaacagctatttagcttggtatttacagaggcaaggtaagagcccccagctgctcgtgtacaacgccaagactttaaccgagggcgtgccttctcgtttcagcggcagcggaagcggcacccagttctctttaaagattaacagcctccagcccgaggacttcggcagctactactgccagcaccactacggccctccccccacatttggcggcggcacaaagctcgaaatcaagggcggaggtgggtcgggtggcggcggatctcagtgcaacgcgccggaatggctgccgtttgcgcgcccgaccaacctgaccgatgaatttgaatttccgattggcacctatctgaactatgaatgccgcccgggctatagcggccgcccgtttagcattatttgcctgaaaaacagcgtgtggaccggcgcgaaagatcgctgccgccgcaaaagctgccgcaacccgccggatccggtgaacggcatggtgcatgtgattaaaggcattcagtttggcagccagattaaatatagctgcaccaaaggctatcgcctgattggcagcagcagcgcgacctgcattattagcggcgataccgtgatttgggataacgaaaccccgatttgcgatcgcattccgtgcggcctgccgccgaccattaccaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaaaccgtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacgcggcgcattggagcaccaaaccgccgatttgccagcgcattccgtgcggcctgccgccgaccattgcgaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattatcgagggcaggcatcaccaccatcaccactga

SEQ ID NO: 51 - protein, 926 aa, Artificial Sequence
  Pselectin2.3scFv-CR1(1-10) Protein
MSVPTQVLGLLLLWLTDARCQIQLVLSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSGGGGSGGGGSGGGGSDIQMTQSPASLSASVGETVTITCRTSDNINSYLAWYLQRQGKSPQLLVYNAKTLTEGVPSRFSGSGSGTQFSLKINSLQPEDFGSYYCQHHYGPPPTFGGGTKLEIKGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIIEGRHHHHHH

SEQ ID NO: 52 - DNA, 4131 bp, Artificial Sequence
  Pselectin2.3scFv-CR1(1-17) DNA
atgtccgtgcccacccaagtgctgggtttattattactgtggctgaccgatgccagatgccagatccagctggtgctgagcggccccgaactgaagaaacccggcgagagcgtcaagatctcttgtaaggccagcggctacaccttcaccacctacggcatgtcttgggtgaagcaagcccccggcaagggtttaaagtggatgggctggatcaacaccagctccggcgtgcctacatacgccgacgactttaagggtcgtttcgccttctctttagagacctccgcctccaccgcctatttacagatcaacaatttaaagaacgaggacaccgccacctacttttgcgctcgtggcggaggctactacggagcctactacttctactattggggccaaggtacaacactgaccgtgtcttctggtggcggcggcagcggcggtggcggctctggcggtggtggcagcgatattcagatgacccagtcccccgcttctttaagcgctagcgtgggagagaccgtgaccatcacttgtagaaccagcgataacatcaacagctatttagcttggtatttacagaggcaaggtaagagcccccagctgctcgtgtacaacgccaagactttaaccgagggcgtgccttctcgtttcagcggcagcggaagcggcacccagttctctttaaagattaacagcctccagcccgaggacttcggcagctactactgccagcaccactacggccctccccccacatttggcggcggcacaaagctcgaaatcaagggcggaggtgggtcgggtggcggcggatctcagtgcaacgcgccggaatggctgccgtttgcgcgcccgaccaacctgaccgatgaatttgaatttccgattggcacctatctgaactatgaatgccgcccgggctatagcggccgcccgtttagcattatttgcctgaaaaacagcgtgtggaccggcgcgaaagatcgctgccgccgcaaaagctgccgcaacccgccggatccggtgaacggcatggtgcatgtgattaaaggcattcagtttggcagccagattaaatatagctgcaccaaaggctatcgcctgattggcagcagcagcgcgacctgcattattagcggcgataccgtgatttgggataacgaaaccccgatttgcgatcgcattccgtgcggcctgccgccgaccattaccaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaaaccgtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacgcggcgcattggagcaccaaaccgccgatttgccagcgcattccgtgcggcctgccgccgaccattgcgaacggcgattttattagcaccaaccgcgaaaactttcattatggcagcgtggtgacctatcgctgcaacccgggcagcggcggccgcaaagtgtttgaactggtgggcgaaccgagcatttattgcaccagcaacgatgatcaggtgggcatttggagcggcccggcgccgcagtgcattattccgaacaaatgcaccccgccgaacgtggaaaacggcattctggtgagcgataaccgcagcctgtttagcctgaacgaagtggtggaatttcgctgccagccgggctttgtgatgaaaggcccgcgccgcgtgaaatgccaggcgctgaacaaatgggaaccggaactgccgagctgcagccgcgtgtgccagccgccgccggatgtgctgcatgcggaacgcacccagcgcgataaagataactttagcccgggccaggaagtgttttatagctgcgaaccgggctatgatctgcgcggcgcggcgagcatgcgctgcaccccgcagggcgattggagcccggcggcgccgacctgcgaagtgaaaagctgcgatgattttatgggccagctgctgaacggccgcgtgctgtttccggtgaacctgcagctgggcgcgaaagtggattttgtgtgcgatgaaggctttcagctgaaaggcagcagcgcgagctattgcgtgctggcgggcatggaaagcctgtggaacagcagcgtgccggtgtgcgaacagattttttgcccgagcccgccggtgattccgaacggccgccataccggcaaaccgctggaagtgtttccgtttggcaaagcggtgaactatacctgcgatccgcatccggatcgcggcaccagctttgatctgattggcgaaagcaccattcgctgcaccagcgatccgcagggcaacggcgtgtggagcagcccggcgccgcgctgcggcattctgggccattgccaggcgccggatcattttctgtttgcgaaactgaaaacccagaccaacgcgagcgattttccgattggcaccagcctgaaatatgaatgccgcccggaatattatggccgcccgtttagcattacctgcctggataacctggtgtggagcagcccgaaagatgtgtgcaaacgcaaaagctgcaaaaccccgccggatccggtgaacggcatggtgcatgtgattaccgatattcaggtgggcagccgcattaactatagctgcaccaccggccatcgcctgattggccatagcagcgcggaatgcattctgagcggcaacaccgcgcattggagcaccaaaccgccgatttgccagcgcattccgtgcggcctgccgccgaccattgcgaacggcgattttattagcaccaaccgcgaaaactttcattatggcagcgtggtgacctatcgctgcaacctgggcagccgcggccgcaaagtgtttgaactggtgggcgaaccgagcatttattgcaccagcaacgatgatcaggtgggcatttggagcggcccggcgccgcagtgcattattatcgagggcaggcatcaccaccatcaccactga

SEQ ID NO: 53 - protein, 1376 aa, Artificial Sequence
  Pselectin2.3scFv-CR1(1-17) Protein
MSVPTQVLGLLLLWLTDARCQIQLVLSGPELKKPGESVKISCKASGYTFTTYGMSWVKQAPGKGLKWMGWINTSSGVPTYADDFKGRFAFSLETSASTAYLQINNLKNEDTATYFCARGGGYYGAYYFYYWGQGTTLTVSSGGGGSGGGGSGGGGSDIQMTQSPASLSASVGETVTITCRTSDNINSYLAWYLQRQGKSPQLLVYNAKTLTEGVPSRFSGSGSGTQFSLKINSLQPEDFGSYYCQHHYGPPPTFGGGTKLEIKGGGGSGGGGSQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIIEGRHHHHHH

(19) United States
(12) Patent Application Publication
(10) Pub. No.: US 2021/0371534 A1
(43) Pub. Date: Dec. 2, 2021

(54) Recombinant Fusion Proteins Targeting P-selectin, and Methods of Use Thereof for Treating Diseases and Disorders

(71) Applicants: MUSC Foundation for Research Development, Charleston, SC (US); The United States Government as Represented by the Department of Veterans Affairs, Washington, DC (US); Universitat De Barcelona, Barcelona (ES)

(72) Inventors: Stephen Tomlinson, Charleston, SC (US); Ali Alawieh, Atlanta, GA (US); Pablo Engel, Barcelona (ES)

(21) Appl. No.: 17/335,355

(22) Filed: Jun. 1, 2021

Related U.S. Application Data
(60) Provisional application No. 63/032,934, filed on Jun. 1, 2020.
(60) Provisional application No. 63/149,725, filed on Feb. 16, 2021.

(51) Int. Cl. C07K 16/28; C07K 14/705; A61P 9/10

(52) U.S. Cl. CPC C07K 16/2854; C07K 14/70596; A61P 9/10; A61K 2039/505; C07K 2317/76; C07K 2317/622; C07K 2317/92; C07K 2319/00

(57) ABSTRACT

The present invention describes compositions and methods for targeting complement inhibition to sites of p-selectin expression, and compositions for inhibiting p-selectin and complement.

### CROSS-REFERENCE TO RELATED APPLICATIONS

This application claims priority to U.S. Provisional Patent Application No. 63/032,934, filed Jun. 1, 2020, and to U.S. Provisional Patent Application No. 63/149,725, filed Feb. 16, 2021, the contents of each of which are incorporated by reference herein in their entirety.

### STATEMENT REGARDING FEDERALLY SPONSORED RESEARCH OR DEVELOPMENT

This invention was made with government support under BX004256 awarded by the Department of Veterans Affairs and under 1U01A132894 and 1I01RX001141 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The present application hereby incorporates by reference the entire contents of the text file named “206085-0087-00US_SequenceListing.txt” in ASCII format. The text file containing the Sequence Listing of the present application was created on May 27, 2021 and is 100,749 bytes in size.

### BACKGROUND OF THE INVENTION

Reconstructive transplantation utilizing vascularized composite (VC) allografts has evolved over the past two decades and has the potential to benefit a wide range of patients including those with congenital anomalies, traumatic injuries, or those needing complex reconstruction following malignant tumor resection. Undeniably, these transplants have the capability to restore appearance, structure, and function to lost or devitalized organs while reducing the donor site morbidity of autologous free tissue transfers and alleviate the need for multiple revisions and/or hospitalizations that may occur with conventional free tissue transfers. The most common vascularized composite allotransplantation (VCA) procedures performed include hand transplantation, face transplantation, and abdominal wall transplantation (Gorantla et al., 2017, Anesthesia and Perioperative Care for Organ Transplantation, Springer New York; 539-552). To date, there have been over 100 hand transplants, 40 full or partial face transplants, and 38 full-thickness and 6 partial-thickness abdominal wall transplants worldwide (Shores et al., 2015, Plast Reconstr Surg, 135(2):351e-360e; Caterson et al., 2018, J Craniofac Surg, 29(4):820-822; Giele et al., 2016, Curr Opin Organ Transplant, 21(2):159-164). Despite many advances in reconstructive transplantation, the clinical benefit to patients remains limited due to the requirement of high-dose, lifelong, multidrug immunosuppression, initial graft injury following ischemia-reperfusion, and the life-improving rather than life-saving nature of these transplants.

The alloimmune response elicited by VC allografts is even more robust than other types of SOT due to the multiple tissue types transplanted and the high immunogenicity of the skin (Chadha et al., 2014, Curr Opin Organ Transplant. 2014; 19(6):566-572). Currently, immunosuppression in VCA is taken from experience with other SOT and there is no standardized regimen (Howsare et al., 2017, Curr Opin Organ Transplant, 22(5):463-469; Kaufman et al., 2019, Am Surg. 2019; 85(6):631-637). The majority of VCAs undergo an episode of acute rejection (Petruzzo et al., 2010, Transplantation, 90(12):1590; Fischer et al., 2014, Curr Opin Organ Transplant, 19(6):531-544). This obviates the need for continued research to optimize immunosuppressive regimens in VCA due to their unique characteristics. While many conventional immunosuppressive regimens exist that target that adaptive immune system, few therapies exist that target the initiating events of the alloimmune response, namely the innate response of complement activation to IRI.

The initial graft injury as a result of ischemia-reperfusion (TR) is an unavoidable consequence of all solid organ transplantation (SOT) and is inextricably linked to the alloimmune response. Ischemia-reperfusion injury (IRI) is initiated by circulating IgM antibodies that bind to neoepitopes or damage-associated molecular patterns (DAMPs) and activate the complement cascade (Ioannou et al., 2011, Clin Immunol, 141(1):3-14), which further primes the adaptive immune response towards the engrafted tissue (Sacks et al., 2012, Nat Rev Immunol, 12(6):431-442). Neutrophil recruitment is another important mediator of injury following IR and is facilitated by adhesion molecules, namely p-selectin, which are up-regulated by complement activation in IRI and allow for rolling of leukocytes along the endothelium (Atkinson et al., 2006, J Immunol, 177(10):7266-7274).

P-selectin is a cell surface glycoprotein that is expressed and upregulated by endothelial cells during inflammation and injury. P-selectin binds a mucin-like glycoprotein (PSGL) on the surface of myeloid cells including neutrophils and macrophages in addition to platelets. Binding then triggers the adhesion of inflammatory cells to the endothelium and subsequent activation of the cells and infiltration of local tissue. This phenomenon is implicated in several auto-immune and inflammatory diseases including among others stroke, traumatic brain injury, ischemia and reperfusion injury, and transplantation. At the same time, complement activation is a major trigger of pathology in many diseases and disease states. In fact, complement activation on the surface of inflamed endothelium triggers a wide range of immune and inflammatory cascades that includes activation of immune cells, damage to tissue, release of cytokines, and expression of P-selectin on the endothelium.

Complement is the collective term for a series of blood proteins that constitute a major effector mechanism of the immune system. The complement system plays an important role in the pathology of many autoimmune, inflammatory and ischemic diseases. Inappropriate complement activation and its deposition on host cells can lead to complement-mediated lysis and/or injury of cells and target tissues, as well as tissue destruction due to the generation of powerful mediators of inflammation. Key to the activity of the complement system is the covalent attachment of processed protein fragments derived from a serum protein, complement C3, to tissue sites of complement activation. This unusual property is due to the presence of a thioester bond in C3 that, when cleaved during C3 activation, converts C3 to a form designated C3b which can then utilize ester or amide bonds to link to cell and tissue-attached molecules. Once C3b is covalently attached, it is rapidly processed to the iC3b, C3dg and C3d forms, each of which remain covalently attached to the target tissue site. This process results in the “marking” of the tissue as one in which an inflammatory injury or other complement-related process is underway.

Complement can be activated by any of three pathways: the classical, lectin and alternative pathways. The classical pathway is activated through the binding of the complement system protein C1q to antigen-antibody complexes, pentraxins or apoptotic cells. The pentraxins include C-reactive protein and serum amyloid P component. The lectin pathway is initiated by binding of microbial carbohydrates to mannose-binding lectin or by the binding of ficolins to carbohydrates or acetylated molecules.

The alternative pathway is activated on surfaces of pathogens that have neutral or positive charge characteristics and do not express or contain complement inhibitors. This results from the process termed ‘tickover’ of C3 that occurs spontaneously, involving the interaction of conformationally altered C3 with factor B, and results in the fixation of active C3b on pathogens or other surfaces. The alternative pathway can also be initiated when certain antibodies block endogenous regulatory mechanisms, by IgA-containing immune complexes, or when expression of complement regulatory proteins is decreased. In addition, the alternative pathway is activated by a mechanism called the ‘amplification loop’ when C3b that is deposited onto targets via the classical or lectin pathway, or indeed through the tickover process itself, binds factor B. See Muller-Eberhard (1988) Ann. Rev. Biochem. 57:321. For example, Holers and colleagues have shown that the alternative pathway is amplified at sites of local injury when inflammatory cells are recruited following initial complement activation. Girardi et al, J. Clin. Invest. 2003, 1 12: 1644. Dramatic complement amplification through the alternative pathway then occurs through a mechanism that involves either the additional generation of injured cells that fix complement, local synthesis of alternative pathway components, or more likely because infiltrating inflammatory cells that carry preformed C3 and properdin initiate and/or greatly increase activation specifically at that site.

Alternative pathway amplification is initiated when circulating factor B binds to activated C3b. This complex is then cleaved by circulating factor D to yield an enzymatically active C3 convertase complex, C3bBb. C3bBb cleaves additional C3 generating C3b, which drives inflammation and also further amplifies the activation process, generating a positive feedback loop. Factor H is a key regulator (inhibitor) of the alternative complement pathway activation and initiation mechanisms that competes with factor B for binding to conformationally altered C3 in the tickover mechanism and to C3b in the amplification loop. Binding of C3b to Factor H also leads to degradation of C3b by factor I to the inactive form iC3b (also designated C3bi), thus exerting a further check on complement activation. Factor H regulates complement in the fluid phase, circulating at a plasma concentration of approximately 500 μg/ml, but its binding to cells is a regulated phenomenon enhanced by the presence of a negatively charged surface as well as fixed C3b, iC3b, C3dg or C3d. Jozsi et al, Histopathol. (2004) 19:251-258.

Complement activation, C3 fragment fixation and complement-mediated inflammation are involved in the etiology and progression of numerous diseases. The down-regulation of complement activation has been shown to be effective in treating several diseases in animal models and in ex vivo studies, including, for example, systemic lupus erythematosus and glomerulonephritis (Y. Wang et al, Proc. Nat'l Acad. Sci. USA (1996) 93:8563-8568), rheumatoid arthritis (Y. Wang et al, Proc. Nat'l Acad. Sci. USA (1995) 92:8955-8959), cardiopulmonary bypass and hemodialysis (C. S. Rinder, J. Clin. Invest. (1995) 96: 1564-1572), hyperacute rejection in organ transplantation (T. J. Kroshus et al, Transplantation (1995) 60: 1194-1202), myocardial infarction (J. W. Homeister et al, J. Immunol. (1993) 150: 1055-1064; H. F. Weisman et al, Science (1990) 249: 146-151), ischemia/reperfusion injury (E. A. Amsterdam et al, Am. J. Physiol. (1995) 268:H448-H457), antibody-mediated allograft rejection, for example, in the kidneys (J. B. Colvin, J. Am. Soc. Nephrol. (2007) 18(4): 1046-56), and adult respiratory distress syndrome (R. Rabinovici et al, J. Immunol. (1992) 149: 1744-1750).

Moreover, other inflammatory conditions and autoimmune/immune complex diseases are also closely associated with complement activation (B. P. Morgan. Eur. J. Clin. Invest. (1994) 24:219-228), including, but not limited to, thermal injury, severe asthma, anaphylactic shock, bowel inflammation, urticaria, angioedema, vasculitis, multiple sclerosis, myasthenia gravis, myocarditis, membranoproliferative glomerulonephritis, atypical hemolytic uremic syndrome, Sjogren's syndrome, renal and pulmonary ischemia/reperfusion, and other organ-specific inflammatory disorders. It is currently uncertain whether complement activation is essential to the pathogenesis and injury of all diseases in which local tissue C3 activation and inflammatory injury occurs; nevertheless, C3 fragment fixation is almost universally found as an associated event.

Previously, a range of targeted complement inhibitors have been characterized that bind locally to sites of injury and protect the inflamed tissue, however, published approaches to target complement inhibitors to the site of injury do not include inhibition of P-selectin binding to its ligand and do not use P-selectin as a targeting vehicle.

Thus, there is a need in the art for methods of treating diseases and disorders associated with P-selectin activity. The present invention provides solutions to these and other problems in the art. The disclosures of all publications, patents, patent applications and published patent applications referred to herein are hereby incorporated herein by reference in their entirety.

### BRIEF SUMMARY OF THE INVENTION

In one embodiment, the invention relates to an antibody or fragment thereof comprising a p-selectin binding domain that specifically binds to p-selectin. In one embodiment, the antibody is selected from the group consisting of a non-blocking anti-p-selectin binding antibody, and an anti-p-selectin blocking antibody.

In one embodiment, the antibody comprises at least one of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO:23. In one embodiment, the antibody comprises at least one of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32 antibody. In one embodiment, the antibody comprises at least one of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44.

In one embodiment, the antibody comprises an amino acid sequence of SEQ ID NO:2, SEQ ID NO:6 or SEQ ID NO:10. In one embodiment, the antibody comprises an amino acid sequence having at least 95% identity to SEQ ID NO:2, SEQ ID NO:6 or SEQ ID NO:10. In one embodiment, the antibody comprises an amino acid sequence comprising at least 80% of the full length sequence of SEQ ID NO:2, SEQ ID NO:6 or SEQ ID NO:10.

In one embodiment, the invention relates to a nucleic acid molecule encoding an antibody or fragment thereof comprising a p-selectin binding domain that specifically binds to p-selectin. In one embodiment, the nucleic acid molecule encodes a non-blocking anti-p-selectin binding antibody. In one embodiment, the nucleic acid molecule encodes an anti-p-selectin blocking antibody.

In one embodiment, the nucleic acid molecule encodes an antibody comprising at least one of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO:23. In one embodiment, the nucleic acid molecule encodes an antibody comprising at least one of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32 antibody. In one embodiment, the nucleic acid molecule encodes an antibody comprising at least one of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44.

In one embodiment, the nucleic acid molecule encodes an antibody comprising an amino acid sequence of SEQ ID NO:2, SEQ ID NO:6 or SEQ ID NO:10. In one embodiment, the nucleic acid molecule encodes an antibody comprising an amino acid sequence having at least 95% identity to SEQ ID NO:2, SEQ ID NO:6 or SEQ ID NO:10. In one embodiment, the nucleic acid molecule encodes an antibody comprising an amino acid sequence comprising at least 80% of the full length sequence of SEQ ID NO:2, SEQ ID NO:6 or SEQ ID NO:10.

In one embodiment, the nucleic acid molecule comprises at least one of a nucleotide sequence of SEQ ID NO:14 encoding a HC CDR1, a nucleotide sequence of SEQ ID NO:16 encoding a HC CDR2, a nucleotide sequence of SEQ ID NO:18 encoding a HC CDR3, a nucleotide sequence of SEQ ID NO:20 encoding a LC CDR1, a nucleotide sequence of SEQ ID NO:22 encoding a LC CDR2, and a nucleotide sequence of SEQ ID NO:24 encoding a LC CDR3. In one embodiment, the nucleic acid molecule comprises at least one of a nucleotide sequence of SEQ ID NO:25 encoding a HC CDR1, a nucleotide sequence of SEQ ID NO:26 encoding a HC CDR2, a nucleotide sequence of SEQ ID NO:27 encoding a HC CDR3, a nucleotide sequence of SEQ ID NO:29 encoding a LC CDR1, a nucleotide sequence of SEQ ID NO:31 encoding a LC CDR2, and a nucleotide sequence of SEQ ID NO:33 encoding a LC CDR3. In one embodiment, the nucleic acid molecule comprises at least one of a nucleotide sequence of SEQ ID NO:35 encoding a HC CDR1, a nucleotide sequence of SEQ ID NO:37 encoding a HC CDR2, a nucleotide sequence of SEQ ID NO:39 encoding a HC CDR3, a nucleotide sequence of SEQ ID NO:41 encoding a LC CDR1, a nucleotide sequence of SEQ ID NO:43 encoding a LC CDR2, and a nucleotide sequence of SEQ ID NO:45 encoding a LC CDR3.

In one embodiment, the nucleic acid molecule comprises a nucleotide sequence of SEQ ID NO:1, SEQ ID NO:5 or SEQ ID NO:9. In one embodiment, the nucleic acid molecule comprises a nucleotide sequence having at least 95% identity to SEQ ID NO:1, SEQ ID NO:5 or SEQ ID NO:9. In one embodiment, the nucleic acid molecule comprises a nucleotide sequence comprising at least 80% of the full length sequence of SEQ ID NO:1, SEQ ID NO:5 or SEQ ID NO:10.

In one embodiment, the invention relates to a fusion molecule comprising a p-selectin binding domain comprising a molecule that specifically binds to p-selectin fused to a cargo domain comprising a complement inhibitor.

In one embodiment, the molecule that specifically binds to p-selectin is a non-blocking anti-p-selectin binding antibody. In one embodiment, the molecule that specifically binds to p-selectin is an anti-p-selectin blocking antibody.

In one embodiment, the p-selectin binding domain comprises an antibody, or fragment thereof, comprising at least one of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO:23. In one embodiment, the p-selectin binding domain comprises an antibody, or fragment thereof, comprising at least one of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32 antibody. In one embodiment, the p-selectin binding domain comprises an antibody, or fragment thereof, comprising at least one of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44.

In one embodiment, the p-selectin binding domain comprises an antibody, or fragment thereof, comprising an amino acid sequence of SEQ ID NO:2, SEQ ID NO:6 or SEQ ID NO:10. In one embodiment, the p-selectin binding domain comprises an antibody, or fragment thereof, comprising an amino acid sequence having at least 95% identity to SEQ ID NO:2, SEQ ID NO:6 or SEQ ID NO:10. In one embodiment, the p-selectin binding domain comprises an antibody, or fragment thereof, comprising an amino acid sequence comprising at least 80% of the full length sequence of SEQ ID NO:2, SEQ ID NO:6 or SEQ ID NO:10.

In one embodiment, the complement inhibitory domain inhibits at least one classical complement pathway, alternative complement pathway or lectin pathway protein. In one embodiment, the complement inhibitory domain inhibits C1, manna binding lectin protease, C3 convertase, C5 convertase, or the membrane attack complex.

In one embodiment, the complement inhibitor is a protein, a peptide, a small molecule, a nucleic acid molecule, an antibody or an antibody fragment.

In one embodiment, the complement inhibitory domain comprises at least one of Factor H (FH), Decay Accelerating Factor (DAF or CD55), Membrane Cofactor Protein (MCP or CD46), Protectin (CD59), Crry (murine equivalent of MCP), Mannose-binding lectin-associated protein of 44 kDa (MAp44), Complement C3b/C4b Receptor 1 (CR1 or CD35), Complement Regulator of the Immunoglobulin Superfamily (CRIg), C4-Binding Protein (C4 bp), OMS721, Eculizumab, Ravulizumab, Coversin, CCX168, IFX 1, CCX168, AMY-101, APL-2, ACH 4471, LPN023, Cemdisiran, C1INH, LFG-316, and plasma serine proteinase inhibitor serpin, or a fragment thereof.

In one embodiment, the fusion molecule comprises an anti-p-selectin antibody, or a variant or a fragment thereof, fused to CR1.

In one embodiment, the fusion molecule comprises an amino acid sequence of SEQ ID NO:47, SEQ ID NO:49, SEQ ID NO:51, or SEQ ID NO:53. In one embodiment, the fusion molecule comprises an amino acid sequence having at least 95% identity to SEQ ID NO:47, SEQ ID NO:49, SEQ ID NO:51, or SEQ ID NO:53. In one embodiment, the fusion molecule comprises an amino acid sequence comprising at least 80% of the full length sequence of SEQ ID NO:47, SEQ ID NO:49, SEQ ID NO:51, or SEQ ID NO:53.

In one embodiment, the invention relates to a nucleic acid molecule encoding a fusion molecule comprising a p-selectin binding domain comprising a molecule that specifically binds to p-selectin fused to a cargo domain comprising a complement inhibitor. In one embodiment, the molecule that specifically binds to p-selectin is a non-blocking anti-p-selectin binding antibody. In one embodiment, the molecule that specifically binds to p-selectin is an anti-p-selectin blocking antibody.

In one embodiment, the nucleic acid molecule encodes a fusion molecule comprising a p-selectin binding domain comprising an antibody, or fragment thereof, comprising at least one of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO:23. In one embodiment, the nucleic acid molecule encodes a fusion molecule comprising a p-selectin binding domain comprising an antibody, or fragment thereof, comprising at least one of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32 antibody. In one embodiment, the nucleic acid molecule encodes a fusion molecule comprising a p-selectin binding domain comprising an antibody, or fragment thereof, comprising at least one of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44.

In one embodiment, the nucleic acid molecule encodes a fusion molecule comprising a p-selectin binding domain comprising an antibody, or fragment thereof, comprising an amino acid sequence of SEQ ID NO:2, SEQ ID NO:6 or SEQ ID NO:10. In one embodiment, the nucleic acid molecule encodes a fusion molecule comprising a p-selectin binding domain comprising an antibody, or fragment thereof, comprising an amino acid sequence having at least 95% identity to SEQ ID NO:2, SEQ ID NO:6 or SEQ ID NO:10. In one embodiment, the nucleic acid molecule encodes a fusion molecule comprising a p-selectin binding domain comprising an antibody, or fragment thereof, comprising an amino acid sequence comprising at least 80% of the full length sequence of SEQ ID NO:2, SEQ ID NO:6 or SEQ ID NO:10.

In one embodiment, the nucleic acid molecule encoding a fusion molecule comprising a p-selectin binding domain comprising an anti-p-selectin antibody, or fragment thereof, comprises at least one of a nucleotide sequence of SEQ ID NO:14 encoding a HC CDR1, a nucleotide sequence of SEQ ID NO:16 encoding a HC CDR2, a nucleotide sequence of SEQ ID NO:18 encoding a HC CDR3, a nucleotide sequence of SEQ ID NO:20 encoding a LC CDR1, a nucleotide sequence of SEQ ID NO:22 encoding a LC CDR2, and a nucleotide sequence of SEQ ID NO:24 encoding a LC CDR3. In one embodiment, the nucleic acid molecule encoding a fusion molecule comprising a p-selectin binding domain comprising an anti-p-selectin antibody, or fragment thereof, comprises at least one of a nucleotide sequence of SEQ ID NO:25 encoding a HC CDR1, a nucleotide sequence of SEQ ID NO:26 encoding a HC CDR2, a nucleotide sequence of SEQ ID NO:27 encoding a HC CDR3, a nucleotide sequence of SEQ ID NO:29 encoding a LC CDR1, a nucleotide sequence of SEQ ID NO:31 encoding a LC CDR2, and a nucleotide sequence of SEQ ID NO:33 encoding a LC CDR3. In one embodiment, the nucleic acid molecule encoding a fusion molecule comprising a p-selectin binding domain comprising an anti-p-selectin antibody, or fragment thereof, comprises at least one of a nucleotide sequence of SEQ ID NO:35 encoding a HC CDR1, a nucleotide sequence of SEQ ID NO:37 encoding a HC CDR2, a nucleotide sequence of SEQ ID NO:39 encoding a HC CDR3, a nucleotide sequence of SEQ ID NO:41 encoding a LC CDR1, a nucleotide sequence of SEQ ID NO:43 encoding a LC CDR2, and a nucleotide sequence of SEQ ID NO:45 encoding a LC CDR3.

In one embodiment, the nucleic acid molecule encoding a fusion molecule comprising a p-selectin binding domain comprising an anti-p-selectin antibody, or fragment thereof, comprises at least one of a nucleotide sequence of SEQ ID NO:1, SEQ ID NO:5 or SEQ ID NO:9. In one embodiment, the nucleic acid molecule encoding a fusion molecule comprising a p-selectin binding domain comprising an anti-p-selectin antibody, or fragment thereof, comprises at least one of a nucleotide sequence having at least 95% identity to SEQ ID NO:1, SEQ ID NO:5 or SEQ ID NO:9. In one embodiment, the nucleic acid molecule encoding a fusion molecule comprising a p-selectin binding domain comprising an anti-p-selectin antibody, or fragment thereof, comprises at least one of a nucleotide sequence comprising at least 80% of the full length sequence of SEQ ID NO:1, SEQ ID NO:5 or SEQ ID NO:9.

In one embodiment, the complement inhibitory domain of the fusion molecule inhibits at least one classical complement pathway, alternative complement pathway or lectin pathway protein. In one embodiment, the complement inhibitory domain inhibits C1, manna binding lectin protease, C3 convertase, C5 convertase, or the membrane attack complex.

In one embodiment, the complement inhibitor is a protein, a peptide, a small molecule, a nucleic acid molecule, an antibody or an antibody fragment.

In one embodiment, the complement inhibitory domain comprises at least one of Factor H (FH), Decay Accelerating Factor (DAF or CD55), Membrane Cofactor Protein (MCP or CD46), Protectin (CD59), Crry (murine equivalent of MCP), Mannose-binding lectin-associated protein of 44 kDa (MAp44), Complement C3b/C4b Receptor 1 (CR1 or CD35), Complement Regulator of the Immunoglobulin Superfamily (CRIg), C4-Binding Protein (C4 bp), OMS721, Eculizumab, Ravulizumab, Coversin, CCX168, IFX 1, CCX168, AMY-101, APL-2, ACH 4471, LPN023, Cemdisiran, C1INH, LFG-316, and plasma serine proteinase inhibitor serpin, or a fragment thereof.

In one embodiment, the nucleic acid molecule encodes a fusion molecule comprising an anti-p-selectin antibody, or a variant or a fragment thereof, fused to CR1.

In one embodiment, the nucleic acid molecule encoding the fusion molecule comprises a nucleotide sequence encoding an amino acid sequence of SEQ ID NO:47, SEQ ID NO:49, SEQ ID NO:51, or SEQ ID NO:53. In one embodiment, the nucleic acid molecule encoding the fusion molecule comprises a nucleotide sequence encoding an amino acid sequence having at least 95% identity to SEQ ID NO:47, SEQ ID NO:49, SEQ ID NO:51, or SEQ ID NO:53. In one embodiment, the nucleic acid molecule encoding the fusion molecule comprises a nucleotide sequence encoding an amino acid sequence comprising at least 80% of the full length sequence of SEQ ID NO:47, SEQ ID NO:49, SEQ ID NO:51, or SEQ ID NO:53.

In one embodiment, the nucleic acid molecule encoding the fusion molecule comprises at least one of SEQ ID NO:46, SEQ ID NO:48, SEQ ID NO: 50 and SEQ ID NO:52. In one embodiment, the nucleic acid molecule encoding the fusion molecule comprises a sequence having at least 95% identity to a nucleotide sequence of SEQ ID NO:46, SEQ ID NO:48, SEQ ID NO: 50 or SEQ ID NO:52. In one embodiment, the nucleic acid molecule encoding the fusion molecule comprises a fragment comprising at least 60% of the full length sequence of a nucleotide sequence of SEQ ID NO:46, SEQ ID NO:48, SEQ ID NO: 50 or SEQ ID NO:52.

In one embodiment, the invention relates to composition comprising an antibody or fragment thereof comprising a p-selectin binding domain that specifically binds to p-selectin. In one embodiment, the composition comprises a non-blocking anti-p-selectin binding antibody. In one embodiment, the composition comprises an anti-p-selectin blocking antibody. In one embodiment, the composition further comprises a pharmaceutically acceptable excipient.

In one embodiment, the invention relates to composition comprising a nucleic acid molecule encoding an antibody or fragment thereof comprising a p-selectin binding domain that specifically binds to p-selectin. In one embodiment, the composition comprises a nucleic acid molecule encoding a non-blocking anti-p-selectin binding antibody. In one embodiment, the composition comprises a nucleic acid molecule encoding an anti-p-selectin blocking antibody. In one embodiment, the composition further comprises a pharmaceutically acceptable excipient.

In one embodiment, the invention relates to a composition comprising a fusion molecule comprising a p-selectin binding domain comprising a molecule that specifically binds to p-selectin fused to a cargo domain comprising a complement inhibitor. In one embodiment, the molecule that specifically binds to p-selectin is a non-blocking anti-p-selectin binding antibody. In one embodiment, the molecule that specifically binds to p-selectin is an anti-p-selectin blocking antibody. In one embodiment, the composition further comprises a pharmaceutically acceptable excipient.

In one embodiment, the invention relates to a composition comprising a nucleic acid molecule encoding a fusion molecule comprising a p-selectin binding domain comprising a molecule that specifically binds to p-selectin fused to a cargo domain comprising a complement inhibitor. In one embodiment, the molecule that specifically binds to p-selectin is a non-blocking anti-p-selectin binding antibody. In one embodiment, the molecule that specifically binds to p-selectin is an anti-p-selectin blocking antibody. In one embodiment, the composition further comprises a pharmaceutically acceptable excipient.

In one embodiment, the invention relates to a method for treating a disease or disorder associated with at least one of p-selectin activity and complement signaling in a subject comprising administering to the subject a therapeutically effective amount of a composition comprising a fusion molecule comprising a p-selectin binding domain comprising a molecule that specifically binds to p-selectin fused to a cargo domain comprising a complement inhibitor, or a nucleic acid molecule encoding the same. In one embodiment, the molecule that specifically binds to p-selectin is a non-blocking anti-p-selectin binding antibody. In one embodiment, the molecule that specifically binds to p-selectin is an anti-p-selectin blocking antibody. In one embodiment, the composition further comprises a pharmaceutically acceptable excipient.

In one embodiment, the disease or disorder is ischemia, reperfusion injury, traumatic brain injury, intracranial hemorrhage, including germinal matrix hemorrhage (GMH) and intraventricular hemorrhage (IVH), post-hemorrhagic hydrocephalus (PHH), coronary artery disease, acute myocardial infarction, stroke, and peripheral artery diseases, allergy, asthma, any autoimmune diseases, celiac disease, glomerulonephritis, hepatitis, inflammatory bowel disease, transplant rejection, coagulopathies, thrombotic disorders, CNS injury, diseases of the CNS and peripheral nervous system, neurodegenerative disorders, ocular disorders, including glaucoma and age-related macular degeneration, infectious disease and pathologies of infectious disease (including but not limited to viral and bacterial infections, systemic organ involvement), blood and clotting disorders or inflammatory diseases and disorders.

### BRIEF DESCRIPTION OF THE DRAWINGS

FIG. 1A and FIG. 1B depict experimental results demonstrating blocking and non-blocking PSelscFv-Crry bind p-selectin and inhibit complement activation in a dose-dependent manner. FIG. 1A depicts exemplary experimental results of a colorimetric binding assay conducted by adding increasing doses of B.PSelscFv-Crry, NB.PSelscFv-Crry, or C3d-Crry to plate bound p-selectin and measuring the optical density at 450 nm. A dose-dependent relationship was observed in the binding of both B.PSelscFv-Crry and NB.PSelscFvCrry constructs, however no binding of C3d-Crry was observed, confirming that binding was mediated by PSelscFv-Crry. FIG. 1B depicts exemplary experimental results of a zymosan bead assay performed to test the ability of each PSelscFv-Crry construct to inhibit complement activation and was compared to a known and validated complement inhibitor (CR2-Crry). Both B.PSelscFv-Crry and NB.PSelscFv-Crry constructs inhibit complement activation in a dose-dependent relationship and have comparable inhibitory functions as CR2-Crry.

FIG. 2A through FIG. 2N depict experimental results demonstrating blocking and non-blocking PSelscFv-Crry reduces IRI in vivo. A murine hindlimb IRI model was used to evaluate the effects of blocking and non-blocking PSelscFv-Crry constructs on IRI. Sham, vehicle, and PSelscFv-Crry injections were performed via tail vein (n=4). Histopathology revealed reduced skeletal muscle injury, edema, and neutrophilic infiltrate in mouse treated with either blocking or non-blocking PSelscFv-Crry as compared to the vehicle control (PBS) and sham-injected mouse (FIG. 2A through FIG. 2F). A greater reduction in injury was seen with the blocking construct (FIG. 2C, FIG. 2D) and at higher doses of 0.5 mg (FIG. 2D, FIG. 2F) compared to 0.25 mg (FIG. 2C, FIG. 2E). Histopathology sections were then stained for C3d by immunohistochemistry to evaluate for complement deposition in each group as compared to the vehicle control (PBS) and sham-injected mice (FIG. 2G-FIG. 2L). The greatest reduction in complement deposition was observed in the B.PSelscFv-Crry group at a dose of 0.5 mg (FIG. 2J). Semi-quantitative histopathology scoring using a scale of 0-5 was performed by two separate trained pathologists and the additive values from each pathologist were graphed for each tissue section (n=5) (FIG. 2M, FIG. 2N). Note the dose-dependent reduction from injury scores in both the blocking and non-blocking PSelscFv-Crry treated animals. (p<0.001 for B.PSelscFv-Crry and p<0.01 for NB.PSelscFv-Crry as compared to controls). No injury was observed in the sham-injected mice.

FIG. 3A through FIG. 3C depicts experimental results demonstrating that NB.PSelscFv-Crry and B.PSelscFv-Crry specifically traffic to site of IRI in vivo. A biodistribution analysis was performed in a mouse hindlimb IRI model by injecting each mouse with 0.25 mg of fluorescently labeled B.PSelscFv-Crry or NB.PSelscFv-Crry following 2 hours of ischemia time. Mice were then imaged with a near infrared Maestro device at 24 hours post-administration. Both B.PSelscFv-Crry and NB.PSelscFv-Crry preferentially targeted to the injured limb as represented both FIG. 3A visually and FIG. 3B graphically (***p<0.001). to vehicle and sham. N=4/group. ***P<0.001. Two-way ANOVA. Bars=mean+/−SEM. FIG. 3C shows the quantification of signal binding at different time points after reperfusion showing that both constructs peak binding at 24 hours after reperfusion. No change in signal was observed in the vehicle group over time. Markers represent mean+/−SEM.

FIG. 4A through FIG. 4E depicts experimental results demonstrating B.PSelscFV-Crry improves hindlimb perfusion at 24 hours post-transplantation in a vascularized composite isograft (VCI) and allograft (VCA). Hindlimb VCI transplanted mice were allotted to either vehicle control (PBS) (n=14), 0.25 mg B.PselscFv-Crry (n=7) (not shown), or 0.5 mg B.PSelscFv-Crry treated groups (n=13) and perfusion was measured in the paw with conventional laser doppler (FIG. 4A). Laser speckle doppler imaging was performed for a subset of mice (vehicle control, n=5, B.PSelscFv-Crry, n=5) and representative images (FIG. 4B) along with graphical representation of paw perfusion is shown (FIG. 4C). Perfusion was significantly improved following a single postoperative administration of 0.5 mg B.PSelscFv-Crry (*p<0.05). Hindlimb VCA transplanted mice were allotted to either vehicle control (PBS) (n=3) or 0.5 mg B.PSelscFv-Crry treated groups (n=4). Representative images perfusion using laser speckle doppler imaging is shown for postoperative days 1, 5, and 9 (FIG. 4D). By postoperative day 9, one control had reached Banff clinical grade 4 rejection. Paw perfusion is also represented graphically (FIG. 4E). A single postoperative dose of 0.5 mg B.PSelscFv-Crry significantly improved hindlimb perfusion measured at day 9 post-transplantation (*p<0.05).

FIG. 5A and FIG. 5B depicts experimental results demonstrating B.PSelscFv-Crry reduces graft injury at 24 hours post-transplantation in a vascularized composite isograft (VCI). Representative histology images of muscle and vasculature from vehicle controls and 0.5 mg B.PSelscFv-Crry treated recipients are shown (FIG. 5A). Note the muscle necrosis (arrows) and neutrophil infiltrates (arrows) in controls that are largely absent in 0.5 mg B.PSelscFv-Crry treated animals. Quantification of injury using a histologic injury score on a scale from 0-4 shows a significant reduction in injury in both the muscle and skin of the 0.5 mg B.PSelscFv-Crry groups as compared to the control groups (FIG. 5B) (#p<0.05).

FIG. 6A and FIG. 6B depicts experimental results demonstrating Single dose of 0.5 mg B.PSelscFv-Crry post-transplantation improves hindlimb vascularized composite allograft (VCA) survival. Allograft survival for orthotopic hindlimb VCA mice was classified as days until Banff clinical grade 4 rejection was reached. A survival curve was generated comparing the vehicle control and 0.5 mg B.PSelscFv-Crry treated groups (FIG. 6A). A single postoperative dose of 0.5 mg B.PSelscFv-Crry significantly improved hindlimb allograft survival (p<0.05). Representative gross images of hindlimb VCA transplanted mice are shown at days 1 and 9 for both the vehicle control group and 0.5 mg B.PSelscFv-Crry group with one control at Banff clinical grade 4 rejection and one treated mouse at Banff clinical grade 1 rejection by day 9 (FIG. 6B).

FIG. 7, comprising FIG. 7A through FIG. 7F, depicts experimental results demonstrating representative images of immunohistochemical detection of myeloperoxidase (MPO) in murine hind limb muscle tissue sections after 2 hours of ischemia and 24 hours of reperfusion. No immunostaining was detected in control PBS-treated mice with isotype control Ab (FIG. 7A). Immunostaining of specimens from mice treated with different doses of Psel.B and Psel-NB mice (FIG. 7C, FIG. 7D, FIG. 7E, and FIG. 7F) showed significantly less MPO-positive cells in muscle tissue than from PBS group (FIG. 7B). The black arrows indicate examples of MPO-positive cells. Original magnification×400.

FIG. 8 depicts experimental results demonstrating a semiquantitative analysis of MPO+ cells. MPO positive cells per 400× field after hindlimb IRI and treatment with different doses of Psel-B and Psel-NB. Pairwise comparisons between hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05), hindlimb IRI+Psel.B (0.25 mg) vs. hindlimb IRI+Psel.B (0.5 mg) (p<0.05), hindlimb IRI+Psel.B (0.5 mg) vs. hindlimb IRI+Psel.NB (0.5 mg) (p<0.05) and hindlimb IRI+Psel.NB (0.25 mg) vs. hindlimb IRI+Psel.NB (0.5 mg) (p<0.05). Differences between hindlimb IRI+Psel.B (0.25 mg) vs. hindlimb IRI+Psel.NB (0.25 mg) and hindlimb IRI+PBS vs. hindlimb IRI+Psel.NB (0.25 mg) were not significant.

FIG. 9 depicts experimental results demonstrating a laser speckle blood flow analysis. Time course of the recovery of blood flow as the ratio of the ligated to non-ligated hindlimb in hindlimb IRI after treatment with different doses of Psel-B and Psel-NB at different time points after 2 hours of ischemia followed by reperfusion. At 6 hours after reperfusion pairwise comparisons between hindlimb IRI+PBS vs. hindlimb IRI+Psel.B (0.5 mg) (p<0.05) and hindlimb IRI+PBS vs. hindlimb IRI+Psel.NB (0.5 mg) (p<0.05). Differences between the other comparisons were not significant. At 24 hours after reperfusion pairwise comparisons hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05),hindlimb IRI+Psel.NB (0.25 mg) vs. hindlimb IRI+Psel.NB (0.5 mg) (p<0.05) and hindlimb IRI+Psel.B (0.25 mg) vs. hindlimb IRI+Psel.NB (0.25 mg) (p<0.05). Differences between the other comparisons were not significant.

FIG. 10 depicts experimental results demonstrating a time course of recovery of blood flow in hindlimb IRI after treatment with different doses of Psel-B and Psel-NB at different time point after 2 hours of ischemia and followed by reperfusion. At 6h after reperfusion pairwise comparisons between hindlimb IRI+PBS vs. hindlimb IRI+Psel.B (0.25 mg) (p<0.05), hindlimb IRI+PBS vs. hindlimb IRI+Psel.B (0.5 mg) (p<0.05) and hindlimb IRI+PBS vs. hindlimb IRI+Psel.NB (0.5 mg) (p<0.05). Differences between the other comparisons were not significant. At 24 hours after reperfusion pairwise comparisons hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05),hindlimb IRI+Psel.NB (0.25 mg) vs. hindlimb IRI+Psel.NB (0.5 mg) (p<0.05) and hindlimb IRI+Psel.B (0.25 mg) vs. hindlimb IRI+Psel.NB (0.25 mg) (p<0.05). Differences between the other comparisons were not significant.

FIG. 11 depicts experimental results demonstrating that bleeding time was measured in hindlimb IRI after treatment with different doses of Psel-B and Psel-NB following 2 hours of ischemia and 6 hours reperfusion. Pairwise comparisons hindlimb IRI+PBS vs. hindlimb IRI+Psel.B (0.5 mg) (p<0.05), hindlimb IRI+Psel.B (0.25 mg) vs. hindlimb IRI+Psel.B (0.5 mg) (p<0.05) and hindlimb IRI+Psel.B (0.5 mg) vs. hindlimb IRI+Psel.NB (0.5 mg) (p<0.05). Differences between the other comparisons were not significant.

FIG. 12, comprising FIG. 12A through FIG. 12C, depicts experimental results demonstrating in-vivo binding to brain after traumatic brain injury. Mice were subjected to traumatic brain injury (moderately severe injury) using the controlled cortical impact model involving the right hemisphere. At 2 hours after brain injury, either Psel2.12-Crry or Psel2.3-Crry that are fluorescently labeled were administered via tail-vein injections at 10 mg/kg dose. Brains were extracted at 24 hours and imaged ex-vivo to determine target localization. FIG. 12A depicts heatmaps of ex-vivo brains from different treatment group showing the signal of the construct in hot colors. Both Psel-Crry constructs targeted specifically to the right hemisphere (site of brain trauma) and minimal binding was observed in the contralateral hemisphere or in sham or vehicle animals. FIG. 12B and FIG. 12C are graphs which represent quantification of signal observed in FIG. 12A, and demonstrate significantly higher binding in the ipsilateral hemisphere (right) compared to left in animals subjected to brain trauma, and significantly higher binding in right hemisphere of brain trauma animals compared to sham. Similar pattern was observed for both inhibitors. N=4-5/group. Two-way ANOVA used for comparisons. ***P<0.001. Bars represent mean+/−SEM.

FIG. 13, comprising FIG. 13A through FIG. 13D, depicts experimental results demonstrating in-vivo binding to brain after stroke. Mice were subjected to stroke using the transient middle cerebral artery ischemia model involving the right hemisphere. At 2 hours after brain injury, either Psel2.12-Crry or Psel2.3-Crry that are fluorescently labeled were administered via tail-vein injections at 10 mg/kg dose. Live animal in-vivo imaging was performed at 24 hours and brains were extracted and imaged ex-vivo to determine target localization at 72 hours. FIG. 13A and FIG. 13C depict heatmaps of animals from different treatment group showing the signal of the construct in hot colors. Both Psel-Crry constructs targeted specifically to the brain (site of stroke) and minimal binding was observed in the rest of the body or in vehicle-treated animals. FIG. 13B and FIG. 13D depict heatmaps of ex-vivo brains from different treatment group showing the signal of the construct in hot colors. Both Psel-Crry constructs targeted specifically to the right hemisphere (site of brain trauma) and minimal binding was observed in the contralateral hemisphere or in sham or vehicle animals.

FIG. 14 depicts experimental results demonstrating acute neuroprotection by Psel 2.12 following stroke. Stroke was induced in mice, and animals were assessed for neurological recovery at 24 hours after stroke using the neurological deficit score (0-4) with 4 being the worst score. The score is used to mimic the deficit scores used in human stroke. Animals treated with Psel2.12-Crry had significant reduction in neurological deficit scores compared to vehicle. Mann-Whiteny test used. N=6 (vehicle), 10 (Psel2.12-Crry). *P<0.05. Median and range are shown.

FIG. 15 depicts the study design of experiments evaluate the effect of p-selectin-targeted complement inhibitors following germinal matrix hemorrhage (GMH).

FIG. 16 depicts images of histological analysis and infarct grading of GMH brains.

FIG. 17 depicts the results of example experiments investigating the rate of post-hemorrhagic hydrocephalus as assessed by Nissl histology in vehicle, P-selectin 2.12 and P-selectin 2.3 treated animals.

FIG. 18 depicts the results of example experiments, demonstrating the Nissl histology results for ventricular volume and infarct lesion.

FIG. 19 depicts the results of example experiments using ultrasonic vocalization testing (USV).

FIG. 20, comprising FIG. 20A through 15C, depicts a representative experimental evaluation of the binding affinity of mouse antigen P-Selectin to B.PselscFv-Crry and NB. PselscFv-Crry. SPR binding assays were performed to measure binding affinity KD and kinetic parameters ka, kd of B.PselscFv-Crry and NB. PselscFv-Crry. The ligand is P-Selectin-His tag (mouse) which was printed onto the chip. The analytes are B.PselscFv-Crry and NB. PselscFv-Crry. After data collection with SPR and kinetics fitting and analysis, the KD, ka, and kd were calculated. FIG. 20A depicts exemplary kinetics fitting curves of P-Selectin to B.PselscFv-Crry. FIG. 20B depicts exemplary kinetics fitting curves of P-Selectin to NB.PselscFv-Crry. FIG. 20C depicts the equilibrium dissociation constant (KD Value) of B.PselscFv-Crry as 3.33×10-7 M. (Ka=4.59×103 M-1·s-1, Kd=1.53×10-3s-1) and the equilibrium dissociation constant (KD Value) of NB. PselscFv-Crry as 6.30×10-7 M. (Ka=2.84×103 M-1 s-1,Kd=1.79×10-3s-1).

FIG. 21, comprising FIG. 21A through 16B, depicts exemplary results demonstrating that B.PSelscFv-Crry and NB.PSelscFv-Crry inhibit complement activation in a dose-dependent manner and bind human P-selectin antigen. FIG. 21A depicts exemplary results of zymosan bead assays performed to test the ability of each PSelscFv-Crry construct to inhibit complement activation. Both B.PSelscFv-Crry and NB.PSelscFv-Crry constructs inhibit complement activation in a dose-dependent relationship. FIG. 21B depicts exemplary results demonstrating a dose-dependent relationship in the binding of both B.PSelscFv-Crry and NB.PSelscFvCrry constructs to P-selectin. However, no binding of C2-Crry was observed, confirming that binding was mediated by PSelscFv-Crry.

FIG. 22, comprising FIG. 22A through 17F, depicts exemplary results demonstrating that B.PSelscFv-Crry (B.PSel) and NB.PSelscFv-Crry (NB.PSel) reduce injury associated with ischemia-reperfusion in vivo. A murine hindlimb IRI model was used to evaluate the effects of B.PSel and NB.PSel constructs on IRI. Sham (n=1), vehicle control (PBS, n=5), 0.25 mg B.PSel (n=5), 0.5 mg B.PSel (n=5), 0.25 mg NB.PSel (n=5), and 0.5 mg NB.PSel (n=5) injections were performed via tail vein. FIG. 22A depicts exemplary H&E stained tissue sections. H&E histopathology revealed reduced skeletal muscle injury, edema, and neutrophilic infiltrate in mice treated with either B.PSel or NB.PSel as compared to the vehicle control (PBS) and sham-injected mouse in a dose-dependent manner. Two separate trained pathologists performed histopathology scoring using a semi-quantitative analysis on a scale of 0-5 and the additive values from each pathologist were graphed for each H&E tissue section. FIG. 22D depicts exemplary results demonstrating a dose-dependent reduction in injury with a significant reduction in injury after administration of 0.5 mg NB.PSel (**p<0.01), 0.25 mg B.PSel (***p<0.05), and 0.5 mg B.PSel (****p<0.01). No injury was seen in the sham injected mice. FIG. 22B depicts exemplary histopathology sections that were then stained for C3d by immunohistochemistry (IHC) to evaluate for complement deposition in each group as compared to the vehicle control (PBS) and sham-injected mice. FIG. 22E depicts exemplary results demonstrating that the greatest reduction in complement deposition was observed in the 0.5 mg B.PSel group, although a dose-dependent relationship in C3d deposition was seen. Quantitative analysis was performed using ImageJ to measure the mean fluorescence intensity. Each dose of either B.PSel or NB.PSel resulted in a significant reduction in C3d deposition within the muscle following IRI (*, **, ***, ****p<0.01) as compared to the vehicle control. FIG. 22C depicts exemplary myeloperoxidase (MPO) IHC stained tissue sections. FIG. 22F depicts exemplary results demonstrating that both B.PSel and NB.PSel administration resulted in a reduction in MPO within the muscle in a dose-dependent relationship. Quantification was performed by assessing the amount of MPO-positive cells present per 400× field. A significant reduction in MPO-positive cells was seen at 0.5 mg NB.PSel (**p<0.01), 0.25 mg B.PSel (***p<0.01), and 0.5 mg B.PSel (***p<0.01) as compared to the vehicle control. No MPO-positive cells were observed in the sham-injected mice.

FIG. 23, comprising FIG. 23A through 18D, depicts exemplary results demonstrating that B.PSelscFv-Crry (B.PSel) reduce P-selectin recruitment and increased bleeding time following Hindlimb IRI. A murine hindlimb IRI model was used to evaluate the effects of B.PSel and NB.PSel constructs on P-selectin. Sham (n=5), vehicle control (PBS, n=5), 0.25 mg B.PSel (n=5), 0.5 mg B.PSel (n=5), 0.25 mg NB.PSel (n=5), and 0.5 mg NB.PSel (n=5) injections were performed via tail vein. As depicted in exemplary images of FIG. 23A, histopathology sections were stained for P-selectin by immunohistochemistry (IHC) to evaluate for deposition of P-selectin in each group as compared to the vehicle control (PBS) and sham-injected mice. FIG. 23B depicts exemplary quantitative analysis performed using ImageJ to measure the mean grey intensity. FIG. 23C and FIG. 23D depict exemplary results of tail bleeding time and bleeding volume, respectively, of mice following hindlimb IRI in the presence of absence of B.PSel and NB.PSel.

FIG. 24, comprising FIG. 24A through 19C, depict exemplary results demonstrating that B.PSelscFv-Crry (B.PSel) and NB.PSelscFv-Crry (NB.PSel) improve perfusion in hindlimb ischemia-reperfusion injury (IRI) model in vivo. Rubber band hindlimb ligation was performed of one hindlimb of each mouse and reperfusion was allowed following 2 hours of ischemic time. Immediately upon reperfusion mice were injected with either vehicle control (PBS) (n=3), NB.PSelscFv-Crry (0.25 mg (n=5) or 0.5 mg (n=5)), or B.PSelscFv-Crry (0.25 mg (n=5) or 0.5 mg (n=5)). FIG. 24A depicts representative laser speckle doppler images of each mouse with arrows indicating ligated hindlimb at various timepoints. FIG. 24B and FIG. 24C depict exemplary quantification using a point on each paw and normalizing to pre-ligation value with conventional doppler measurements (FIG. 24B) and laser speckle doppler measurements (FIG. 24C). Significant improvements in hindlimb perfusion were seen at 6 hours post-reperfusion with all treatment groups using conventional doppler measurements (p<0.05) and with administration of 0.5 mg of NB.PSel (p<0.05), 0.25 mg B.PSel (p<0.01), and 0.5 mg B.PSel (p<0.01) using laser speckle doppler measurements. At 24 hours post-reperfusion, a significant improvement in perfusion was seen following administration with 0.5 mg NB.PSel (#p<0.05), 0.25 mg B.PSel (##p<0.01), and 0.5 mg B.PSel (###p<0.01) using conventional doppler. However, only treatments of 0.25 mg B.PSel (**p<0.01) and 0.5 mg B.PSel (***p<0.01) resulted in significant improvement in perfusion with laser speckle doppler.

FIG. 25, comprising FIG. 25A through 25B, depicts exemplary results of evaluating the serum circulatory half-life of B.PSelscFv-Crry (B.PSel) and NB.PSelscFv-Crry (NB.PSel). B.PSelscFv-Crry or NB.PSelscFv-Crry at a dose of 0.5 mg was administered i.v., and blood samples collected at indicated times for analysis of protein construct levels by anti-P-selectin ELISA. FIG. 25A and FIG. 25B depict exemplary two-phase exponential decay curves that were fitted to data for both constructs and the calculated pharmacokinetic parameters. B.PSelscFv-Crry has a fast half-life of 0.73 h and slow half-life of 28.88 h (FIG. 25A) and NB.PSelscFv-Crry has a fast half-life of 0.29 hours and slow half-life of 13.61 hours (FIG. 25B). Mean±SD, n=3.

FIG. 26, comprising FIG. 26A through 21B, depicts exemplary results demonstrating that B.PSelscFv-Crry and NB.PSelscFv-Crry specifically traffic to site of IRI in vivo. A biodistribution analysis was performed in a mouse hindlimb IRI model by injecting each mouse with 0.25 mg of fluorescently labeled B.PSelscFv-Crry or NB.PSelscFv-Crry following 2 hours of ischemia time. Mice were then imaged with a near infrared Maestro device at 24 hours post-administration. FIG. 26A and FIG. 26B depict exemplary results demonstrating that both B.PSelscFv-Crry and NB.PSelscFv-Crry preferentially targeted to the injured limb as represented both visually (FIG. 26A) and graphically (FIG. 26B; ***p<0.001).

FIG. 27, comprising FIG. 27A through FIG. 27F, depicts exemplary results demonstrating that B.PSelscFv-Crry (B.PSel) reduces graft injury, myeloperoxidase (MPO), and C3d deposition following syngeneic hindlimb transplantation at 6 and 24 hours post-transplantation in a vascularized composite isograft (VCI). Tissue sections from the thigh muscle of each mouse hindlimb were taken at either 6 hours or 24 hours post-transplant in a VCI mouse model. H&E (FIG. 27A and FIG. 27B), C3d immunohistochemistry (IHC) (FIG. 27C and FIG. 27D), and MPO IHC (FIG. 27E and FIG. 27F) staining of these tissue sections are shown with quantification. Muscle from normal B6 mice with no transplant is shown as the sham controls and muscle from VCI transplants injected with PBS at 30 minutes post-transplant with graft harvest at 6 hours and 24 hours are shown as the vehicle controls. FIG. 27A depicts exemplary results demonstrating muscle necrosis and neutrophilic infiltrates in controls that are largely absent in 0.5 mg B.PSelscFv-Crry treated animals seen with the H&E staining. FIG. 27B depicts exemplary quantification of injury using a histologic injury score on a scale from 0-4, which shows a significant reduction in injury in the 0.5 mg B.PSelscFv-Crry treated group as compared to the control group (p<0.05). FIG. 27C depicts exemplary results demonstrating a dose-dependent reduction in C3d observed with immediate post-transplant administration of B.PselscFv-Crry at both 6 hours and 24 hours. FIG. 27D depicts exemplary quantification of C3d staining performed using ImageJ to measure the mean fluorescence intensity. While there was not a significant reduction in C3d deposition at the 6 hour time point with either 0.25 mg (*p=0.36) or 0.5 mg (**p=0.09) of B.PselscFv-Crry or at the 24 hour time point with 0.25 mg B.PselscFv-Crry (***p=0.07), there was a significant reduction in C3d deposition at the 24 hour time point with the 0.5 mg B.PselscFv-Crry dose (****p<0.05). Similarly, as depicted by exemplary results in FIG. 27E, reductions in MPO positive cells were seen following B.PselscFv-Crry administration in a dose-dependent fashion at both 6 hours and 24 hours. FIG. 27F depicts exemplary MPO quantification represented as the number of MPO positive cells per 400× field magnification. A significant reduction in MPO positive cells was observed at 6 hours with 0.25 mg (*p<0.01) and 0.5 mg (**p<0.01) of B.PselscFv-Crry and at 24 hours with only the 0.5 mg dose of B.PSelscFv-Crry (****p<0.05).

FIG. 28, comprising FIG. 28A through 23C, depicts exemplary results demonstrating that B.PSelscFv-Crry improves hindlimb perfusion at 24 hours post-transplantation in a vascularized composite isograft (VCI). Hindlimb VCI transplanted mice were allotted to either vehicle control (PBS) (n=14), 0.25 mg B.PselscFv-Crry (n=7) (not shown), or 0.5 mg B.PSelscFv-Crry treated groups (n=13) and perfusion was measured in the paw with conventional doppler. Laser speckle doppler imaging was performed for a subset of mice (vehicle control, n=5, B.PSelscFv-Crry, n=5) and representative images, as depicted in FIG. 28A, along with exemplary graphical representations of paw perfusion using conventional doppler, as depicted in FIG. 28B and laser speckle doppler, as depicted in FIG. 28C, are shown. Perfusion was significantly improved at 24 hours post-transplantation following a single postoperative administration of 0.5 mg B.PSelscFv-Crry given at 30 minutes post-reperfusion (*p<0.05).

FIG. 29, comprising FIG. 29A through FIG. 29B, depicts exemplary results demonstrating that B.PSelscFv-Crry improves perfusion in vascularized composite allograft (VCA) hindlimb transplantation. Hindlimb VCA transplanted mice were allotted to either vehicle control (PBS) (n=3) or 0.5 mg B.PSelscFv-Crry treated groups (n=4). A single dose of 0.5 mg B.PSelscFv-Crry was administered at 30 minutes post-reperfusion. As depicted in FIG. 29A, laser speckle doppler imaging was performed and representative images of perfusion are shown at pre-transplantation (Pre-Txp), 30 minutes post-reperfusion, and at postoperative days 1, 7, 9, 16, and 18. As depicted in exemplary results of FIG. 29B, paw perfusion is also represented graphically as speckle perfusion index. While a single postoperative dose of 0.5 mg B.PSelscFv-Crry significantly improved hindlimb perfusion measured at day 9 post-transplantation (*p<0.05), no other time points resulted in a significant improvement in perfusion.

FIG. 30, comprising FIG. 30A through FIG. 30B, depicts exemplary results demonstrating that B.PSelscFv-Crry improves survival of hindlimb vascularized composite allografts (VCA). Allograft survival for orthotopic hindlimb VCA mice was classified as days until Banff clinical grade 4 rejection was reached. FIG. 30A depicts representative gross images of hindlimb VCA transplanted mice at pre-transplantation of the recipient (Pre-Txp), 30 minutes post-reperfusion, and at postoperative days 1, 7, 9, 16, and 18. FIG. 30B depicts an exemplary survival curve comparing the vehicle control (PBS) and 0.5 mg B.PSelscFv-Crry treated groups. A single postoperative dose of 0.5 mg B.PSelscFv-Crry significantly improved hindlimb allograft survival (p<0.05).

### DETAILED DESCRIPTION OF THE INVENTION

In one embodiment the invention relates to compositions and methods to target complement inhibition to sites of inflammation. In one embodiment the invention relates to compositions and methods to target a complement inhibitor to sites of P-selectin expression. In one embodiment, the targeting moiety consists of anti-P-selectin Ab fragments including, but not limited to, scFv, Fab, whole Ab or other derivatives, linked to a complement inhibitor. In some embodiments, the P-selectin Abs or derived scFvs may be non-blocking or may have blocking (inhibitory) P-selectin activity.

The types of compositions of the invention have wide potential application for treating injury in general including, but not limited to, ischemia reperfusion injury, inflammation, autoimmunity, alloimmunity, coagulopathies, thrombotic disorders, brain and CNS injuries, neurodegenerative conditions, cancer and any disease/condition in which the adhesion molecule P-selectin is expressed or in which blocking the otherwise normal physiological function of P-selectin provides a therapeutic effect.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

There term “in combination with” is used herein to that the indicated treatments are administered concurrently or that a first treatment is administered sequentially with one or more additional treatment.

The term “diagnosis” refers to a relative probability that a disease (e.g. an autoimmune, inflammatory autoimmune, cancer, infectious, immune, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. an autoimmune, inflammatory autoimmune, cancer, infectious, immune, or other disease), or the likely severity of the disease (e.g., duration of disease). The terms are not intended to be absolute, as will be appreciated by any one of skill in the field of medical diagnostics.

A “disease” is a state of health of an animal wherein the animal cannot maintain homeostasis, and wherein if the disease is not ameliorated then the animal's health continues to deteriorate.

The terms “effective amount” and “pharmaceutically effective amount” or “therapeutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

The term “fusion protein” used herein refers to two or more peptides, polypeptides, or proteins operably linked to each other.

An “individual” is a vertebrate, preferably a mammal, more preferably a human. Mammals include, but are not limited to, farm animals, sport animals, pets, primates, mice and rats. In some embodiments, the individual is human. In some embodiments, the individual is an individual other than human.

The term “inhibit,” as used herein, means to suppress or block an activity or function relative to a control value. Preferably, the activity is suppressed or blocked by 10% compared to a control value, more preferably by 50%, and even more preferably by 95%.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al, J. Biol. Chem. 273(52):35095-35101 (1998).

A nucleotide sequence is “operably linked” when it is placed into a functional relationship with another nucleotide sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in IX SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Twenty amino acids are commonly found in proteins. Those amino acids can be grouped into nine classes or groups based on the chemical properties of their side chains. Substitution of one amino acid residue for another within the same class or group is referred to herein as a “conservative” substitution. Conservative amino acid substitutions can frequently be made in a protein without significantly altering the conformation or function of the protein. Substitution of one amino acid residue for another from a different class or group is referred to herein as a “non-conservative” substitution. In contrast, non-conservative amino acid substitutions tend to modify conformation and function of a protein.

TABLE 1:\
Example of amino acid classification

| | |
|---|---|
| Small/Aliphatic residues: | Gly, Ala, Val, Leu, Ile |
| Cyclic Imino Acid: | Pro |
| Hydroxyl-containing Residues: | Ser, Thr |
| Acidic Residues: | Asp, Glu |
| Amide Residues | Asn, Gln |
| Basic Residues: | Lys, Arg |
| Imidazole Residue: | His |
| Aromatic Residues: | Phe, Tyr, Trp |
| Sulfur-containing Residues: | Met, Cys |

In some embodiments, the conservative amino acid substitution comprises substituting any of glycine (G), alanine (A), isoleucine (I), valine (V), and leucine (L) for any other of these aliphatic amino acids; serine (S) for threonine (T) and vice versa; aspartic acid (D) for glutamic acid (E) and vice versa; glutamine (Q) for asparagine (N) and vice versa; lysine (K) for arginine (R) and vice versa; phenylalanine (F), tyrosine (Y) and tryptophan (W) for any other of these aromatic amino acids; and methionine (M) for cysteine (C) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pKs of these two amino acid residues are not significant. Still other changes can be considered “conservative” in particular environments (see, e.g., BIOCHEMISTRY at pp. 13-15, 2nd ed. Lubert Stryer ed. (Stanford University); Henikoff et al, Proc. Nat'l Acad. Set USA (1992) 89: 10915-10919; Lei et al., J. Biol. Chem. (1995) 270(20): 1 1882-1 1886).

In some embodiments, the non-conservative amino acid substitution comprises substituting any of glycine (G), alanine (A), isoleucine (I), valine (V), and leucine (L) for any of serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of serine (S) and threonine (T) for any of glycine (G), alanine (A), isoleucine (I), valine (V), leucine (L), aspartic acid (D), glutamic acid (E), glutamine (Q), asparagine (N), lysine (K), arginine (R), phenylalanine (F), tyrosine (Y), tryptophan (W), methionine (M), cysteine (C), histidine (H) and proline (P). In some embodiments, the non-conservative amino acid substitution comprises substituting any of aspartic acid (D) and glutamic acid (E) for any of glycine (G), alanine (A), isoleucine (I), valine (V), leucine (L), serine (S), threonine (T), glutamine (Q), asparagine (N), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of glutamine (Q) and asparagine (N) for any of glycine (G), alanine (A), isoleucine (I), valine (V), leucine (L), serine (S), threonine (T), aspartic acid (D), glutamic acid (E), lysine (K), arginine (R), phenylalanine (F), tyrosine (Y), tryptophan (W), methionine (M), cysteine (C), histidine (H), and proline (P). In some embodiments, the non-conservative amino acid substitution comprises substituting any of lysine (K) and arginine (R) for any of glycine (G), alanine (A), isoleucine (I), valine (V), leucine (L), serine (S), threonine (T), aspartic acid (D), glutamic acid (E), glutamine (Q), asparagine (N), phenylalanine (F), tyrosine (Y), tryptophan (W), methionine (M), cysteine (C), histidine (H), and proline (P). In some embodiments, the non-conservative amino acid substitution comprises substituting any of phenylalanine (F), tyrosine (Y), and tryptophan (W) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), methionine (M), cysteine (C), histidine (H), and proline (P). In some embodiments, the non-conservative amino acid substitution comprises substituting any of methionine (M) and cysteine (C) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), histidine (H), and proline (P). In some embodiments, the non-conservative amino acid substitution comprises substituting histidine (H) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), and proline (P). In some embodiments, the non-conservative amino acid substitution comprises substituting proline (P) for any of glycine (G), alanine (A), isoleucine (I), valine (V), leucine (L), serine (S), threonine (T), aspartic acid (D), glutamic acid (E), glutamine (Q), asparagine (N), lysine (K), arginine (R), phenylalanine (F), tyrosine (Y), tryptophan (W), methionine (M), cysteine (C), and histidine (H).

“Polypeptide,” “peptide,” and “protein” are used herein interchangeably and mean any peptide-linked chain of amino acids, regardless of length or post-translational modification. As noted below, the polypeptides described herein can be, e.g., wild-type proteins, biologically-active fragments of the wild-type proteins, or variants of the wild-type proteins or fragments. Variants, in accordance with the disclosure, can contain amino acid substitutions, deletions, or insertions. The substitutions can be conservative or non-conservative. In some embodiments, conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Following expression, the proteins (e.g. antibodies, antigen-binding fragments thereof, conjugates, antibody-conjugates) can be isolated. The term “purified” or “isolated” as applied to any of the proteins described herein (e.g., a conjugate described herein, antibody or antigen-binding fragment thereof described herein) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A “label” or a “detectable moiety” is a composition detectable by spectroscopic, photochemical, biochemical, immunochemical, chemical, magnetic resonance imaging, or other physical means. For example, useful detectable moieties include ³²P, fluorescent dyes, electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, standard superparamagnetic iron oxide (“SSPIO”), SSPIO nanoparticle aggregates, polydisperse superparamagnetic iron oxide (“PSPIO”), PSPIO nanoparticle aggregates, monochrystalline SPIO, monochrystalline SPIO aggregates, monochrystalline iron oxide nanoparticles, monochrystalline iron oxide, other nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Detectable moieties also include any of the above compositions encapsulated in nanoparticles, particles, aggregates, coated with additional compositions, derivatized for binding to a targeting agent (e.g. antibody or antigen binding fragment). Any method known in the art for conjugating an antibody to the label may be employed, e.g., using methods described in Hermanson, Bioconjugate Techniques 1996, Academic Press, Inc., San Diego.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The term “diagnostically acceptable” is used synonymously with “physiologically acceptable” and “pharmacologically acceptable” and refers to diagnostic compositions.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The term “sub-therapeutic” as used herein means a treatment at a dose known to be less than what is known to induce a therapeutic effect.

The term “therapeutic” as used herein means a treatment and/or prophylaxis. A therapeutic effect is obtained by suppression, remission, or eradication of a disease state.

The term “therapeutic agent” use herein refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

This invention describes, in part, therapeutic compositions and methods for targeting complement inhibition to sites of inflammation using p-selectin specific binding. In certain embodiments, the composition of the invention comprises a fusion antibody comprising an anti-p-selectin domain and a complement inhibition domain. In certain embodiments, the anti-p-selectin domain comprises an antibody, or a fragment thereof, that specifically binds to p-selectin. In one embodiment, the antibody, or a fragment thereof, that specifically binds to p-selectin inhibits p-selectin activity. Such an antibody is referred to herein as a “blocking antibody.” In one embodiment, the antibody, or a fragment thereof, that specifically binds to p-selectin does not affect p-selectin activity. Such an antibody is referred to as a “non-blocking antibody.”

In some embodiments, the method comprises administering a composition comprising a p-selectin-targeted complement inhibitor to a subject in need thereof for the treatment of a disease or disorder associated with p-selectin expression. Exemplary diseases and disorders that can be treated using the p-selectin-targeted complement inhibitors of the invention include, but are not limited to, ischemia, reperfusion injury, traumatic brain injury, intracranial hemorrhage, including germinal matrix hemorrhage (GMH) and intraventricular hemorrhage (IVH), post-hemorrhagic hydrocephalus (PHH), coronary artery disease, acute myocardial infarction, stroke, and peripheral artery diseases, allergy, asthma, any autoimmune diseases, celiac disease, glomerulonephritis, hepatitis, inflammatory bowel disease, transplant rejection, coagulopathies, thrombotic disorders, CNS injury, diseases of the CNS and peripheral nervous system, neurodegenerative disorders, ocular disorders, including glaucoma and age-related macular degeneration, infectious disease and pathologies of infectious disease (including but not limited to viral and bacterial infections, systemic organ involvement), blood and clotting disorders and inflammatory diseases and disorders.

In one embodiment, the present invention relates to a composition used to treat a subject that has suffered an ischemia, reperfusion injury, traumatic brain injury, intracranial hemorrhage, including germinal matrix hemorrhage (GMH) and intraventricular hemorrhage (IVH), post-hemorrhagic hydrocephalus (PHH), coronary artery disease, acute myocardial infarction, stroke, and peripheral artery diseases, allergy, asthma, any autoimmune diseases, celiac disease, glomerulonephritis, hepatitis, inflammatory bowel disease, transplant rejection, coagulopathies, thrombotic disorders, CNS injury, diseases of the CNS and peripheral nervous system, neurodegenerative disorders, ocular disorders, including glaucoma and age-related macular degeneration, infectious disease and pathologies of infectious disease (including but not limited to viral and bacterial infections, systemic organ involvement), blood and clotting disorders and inflammatory diseases and disorders.

In one embodiment, the composition modulates signaling of a complement pathway. In some instances, complement pathway is the main pathway, an alternative pathway, or any combination thereof.

### Antibody Compositions

In some embodiments, the invention relates to compositions comprising at least one antibody, or fragment thereof, specific for binding to p-selectin. In one embodiment, the antibody, or fragment thereof, may be used for site-specific delivery of a cargo molecule. For example, in some embodiments, there is provided a fusion protein comprising a p-selectin binding or blocking domain, or a fragment thereof operably linked to cargo domain comprising a cargo molecule. In some embodiments, the cargo molecule is a protein, a peptide, a nucleic acid molecule, an antibody or an antibody fragment. In some embodiments, the cargo molecule is a therapeutic molecule for the treatment of a disease or disorder. In some embodiments, the cargo domain comprises a complement inhibitor.

In one embodiment, the anti-p-selectin antibody of the invention binds to p-selectin, but does not alter the activity of p-selectin. In such an embodiment the anti-p-selectin antibody is a non-blocking antibody. In one embodiment, the fusion molecule of the invention comprises at least one antibody specific for binding to p-selectin which also inhibits the activity of p-selectin. In such an embodiment the anti-p-selectin antibody is a blocking antibody.

In one embodiment, the p-selectin binding domain comprises a non-blocking p-selectin antibody comprising at least one CDR of a heavy chain (HC) CDR1 sequence comprising SEQ ID NO:13, a HC CDR2 sequence comprising SEQ ID NO:15, a HC CDR3 sequence comprising SEQ ID NO:17, a light chain (LC) CDR1 sequence comprising SEQ ID NO:19, a LC CDR2 sequence comprising SEQ ID NO:21, and a LC CDR3 sequence comprising SEQ ID NO:23. In one embodiment, the non-blocking p-selectin antibody comprises 1, 2, 3, 4, 5, or all 6 CDRs as set forth in SEQ ID NO:13, SEQ ID NO:15, SEQ ID NO:17, SEQ ID NO:19, SEQ ID NO:21 and SEQ ID NO:23. In one embodiment, the p-selectin binding domain comprises a non-blocking p-selectin antibody comprising a heavy chain (HC) CDR1 sequence comprising SEQ ID NO:13, a HC CDR2 sequence comprising SEQ ID NO:15, a HC CDR3 sequence comprising SEQ ID NO:17, a light chain (LC) CDR1 sequence comprising SEQ ID NO:19, a LC CDR2 sequence comprising SEQ ID NO:21, and a LC CDR3 sequence comprising SEQ ID NO:23.

In one embodiment, the p-selectin binding domain comprises a non-blocking p-selectin antibody comprising at least one of an HC CDR1 sequence comprising SEQ ID NO:13, a HC CDR2 sequence comprising SEQ ID NO:15, a HC CDR3 sequence comprising SEQ ID NO:17, a LC CDR1 sequence comprising SEQ ID NO:28, a LC CDR2 sequence comprising SEQ ID NO:30, and a LC CDR3 sequence comprising SEQ ID NO:32. In one embodiment, the non-blocking p-selectin antibody comprises 1, 2, 3, 4, 5, or all 6 CDRs as set forth in SEQ ID NO: 13, SEQ ID NO:15, SEQ ID NO:17, SEQ ID NO:28, SEQ ID NO:30 and SEQ ID NO:32. In one embodiment, the p-selectin binding domain comprises a non-blocking p-selectin antibody comprising a HC CDR1 sequence comprising SEQ ID NO:13, a HC CDR2 sequence comprising SEQ ID NO:15, a HC CDR3 sequence comprising SEQ ID NO:17, a LC CDR1 sequence comprising SEQ ID NO:28, a LC CDR2 sequence comprising SEQ ID NO:30, and a LC CDR3 sequence comprising SEQ ID NO:32.

In one embodiment, the p-selectin binding domain comprises a blocking p-selectin antibody comprising at least one of a HC CDR1 sequence comprising SEQ ID NO:34, a HC CDR2 sequence comprising SEQ ID NO:36, a HC CDR3 sequence comprising SEQ ID NO:38, a LC CDR1 sequence comprising SEQ ID NO:40, a LC CDR2 sequence comprising SEQ ID NO:42, and a LC CDR3 sequence comprising SEQ ID NO:44. In one embodiment, the non-blocking p-selectin antibody comprises 1, 2, 3, 4, 5, or all 6 CDRs as set forth in SEQ ID NO:34, SEQ ID NO:36, SEQ ID NO:38, SEQ ID NO:40, SEQ ID NO:42 and SEQ ID NO:44. In one embodiment, the p-selectin binding domain comprises a blocking p-selectin antibody comprising a HC CDR1 sequence comprising SEQ ID NO:34, a HC CDR2 sequence comprising SEQ ID NO:36, a HC CDR3 sequence comprising SEQ ID NO:38, a LC CDR1 sequence comprising SEQ ID NO:40, a LC CDR2 sequence comprising SEQ ID NO:42, and a LC CDR3 sequence comprising SEQ ID NO:44.

In one embodiment, the p-selectin binding domain comprises a non-blocking p-selectin antibody comprising a sequence as set forth in SEQ ID NO:2 or SEQ ID NO:6, or a fragment or variant thereof. In one embodiment, the p-selectin binding domain comprises a p-selectin blocking antibody comprising a sequence as set forth in SEQ ID NO:10, or a fragment or variant thereof.

In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined amino acid sequence. In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of an amino acid sequence of SEQ ID NO:2, SEQ ID NO:6, or SEQ ID NO:10.

In some embodiments, a fragment of an amino acid sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of a defined amino acid sequence. In some embodiments, a fragment of an amino acid sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of SEQ ID NO:2, SEQ ID NO:6, or SEQ ID NO:10.

As used herein, the term “antibody” or “immunoglobulin” refers to proteins (including glycoproteins) of the immunoglobulin (Ig) superfamily of proteins. An antibody or immunoglobulin (Ig) molecule may be tetrameric, comprising two identical light chain polypeptides and two identical heavy chain polypeptides. The two heavy chains are linked together by disulfide bonds, and each heavy chain is linked to a light chain by a disulfide bond. Each full-length Ig molecule contains at least two binding sites for a specific target or antigen.

An anti-p-selectin blocking or non-blocking antibody, or antigen-binding fragment thereof, includes, but is not limited to a polyclonal antibody, a monoclonal fusion proteins, antibodies or fragments thereof, chimerized or chimeric fusion proteins, antibodies or fragments thereof, humanized fusion proteins, antibodies or fragments thereof, deimmunized humfusion proteins, antibodies or fragments thereof, fully humfusion proteins, antibodies or fragments thereof, single chain antibody, single chain Fv fragment (scFv), Fv, Fd fragment, Fab fragment, Fab′ fragment, F(ab′)₂ fragment, diabody or antigen-binding fragment thereof, minibody or antigen-binding fragment thereof, triabody or antigen-binding fragment thereof, domain fusion proteins, antibodies or fragments thereof, camelid fusion proteins, antibodies or fragments thereof, dromedary fusion proteins, antibodies or fragments thereof, phage-displayed fusion proteins, antibodies or fragments thereof, or antibody, or antigen-binding fragment thereof, identified with a repetitive backbone array (e.g. repetitive antigen display).

The immune system produces several different classes of Ig molecules (isotypes), including IgA, IgD, IgE, IgG, and IgM, each distinguished by the particular class of heavy chain polypeptide present: alpha (a) found in IgA, delta (δ) found in IgD, epsilon (ε) found in IgE, gamma (γ) found in IgG, and mu (μ) found in IgM. There are at least five different γ heavy chain polypeptides (isotypes) found in IgG. In contrast, there are only two light chain polypeptide isotypes, referred to as kappa (κ) and lambda (λ) chains. The distinctive characteristics of antibody isotypes are defined by sequences of the constant domains of the heavy chain.

An IgG molecule comprises two light chains (either κ or λ form) and two heavy chains (γ form) bound together by disulfide bonds. The κ and λ forms of IgG light chain each contain a domain of relatively variable amino acid sequences, called the variable region (variously referred to as a “V_{L}-,” “V_{κ}-,” or “V_{λ}-region”) and a domain of relatively conserved amino acid sequences, called the constant region (CL-region). Similarly, each IgG heavy chain contains a variable region (VH-region) and one or more conserved regions: a complete IgG heavy chain contains three constant domains (“C_{H}1-,” “C_{H}2-,” and “C_{H}3-regions”) and a hinge region. Within each VL- or VH-region, hypervariable regions, also known as complementarity-determining regions (“CDR”), are interspersed between relatively conserved framework regions (“FR”). Generally, the variable region of a light or heavy chain polypeptide contains four FRs and three CDRs arranged in the following order along the polypeptide: NH₂-FR1-CDR1-FR2-CDR2-FR3-CDR3-FR4-COOH. Together the CDRs and FRs determine the three-dimensional structure of the IgG binding site and thus, the specific target protein or antigen to which that IgG molecule binds. Each IgG molecule is dimeric, able to bind two antigen molecules. Cleavage of a dimeric IgG with the protease papain produces two identical antigen-binding fragments (“Fab′”) and an “Fc” fragment or Fc domain, so named because it is readily crystallized.

As used throughout the present disclosure, the term “antibody” further refers to a whole or intact antibody (e.g., IgM, IgG, IgA, IgD, or IgE) molecule that is generated by any one of a variety of methods that are known in the art and described herein. The term “antibody” includes a polyclonal antibody, a monoclonal antibody, a chimerized or chimeric antibody, a humanized antibody, a deimmunized human antibody, and a fully human antibody. The antibody can be made in or derived from any of a variety of species, e.g., mammals such as humans, non-human primates (e.g., monkeys, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “epitope” refers to the site on a protein that is bound by an antibody. “Overlapping epitopes” include at least one (e.g., two, three, four, five, or six) common amino acid residue(s).

In one embodiment, the antibody of the invention specifically binds to p-selectin. As used herein, the terms “specific binding” or “specifically binds” refer to two molecules forming a complex that is relatively stable under physiologic conditions. Typically, binding is considered specific when the association constant (Kₐ) is higher than 10⁶ M-1. Thus, an antibody can specifically bind to a target with a Ka of at least (or greater than) 10⁶ (e.g., at least or greater than 10⁷, 10⁸, 10⁹, 10¹⁰, 10¹¹, 10¹², 10¹³, 10¹⁴, or 10¹⁵ or higher) M⁻¹.

In one embodiment, the antibody of the invention specifically binds to p-selectin.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), or enzyme-linked immunosorbent assays (ELISA). See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2nd Edition, Oxford University Press, NY, Oxford; Johne et al. (1993) J. Immunol. Meth. 160: 191-198; Jonsson et al. (1993) Ann. Biol. Clin. 51: 19-26; and Jonsson et al. (1991) Biotechniques 11:620-627. See also, U.S. Pat. No. 6,355,245.

Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

Antibodies can also be assayed using any surface plasmon resonance (SPR)-based assays known in the art for characterizing the kinetic parameters of the interaction of the antibody with its target or epitope. Any SPR instrument commercially available including, but not limited to, BIAcore Instruments (Biacore AB; Uppsala, Sweden); IAsys instruments (Affinity Sensors; Franklin, Mass.); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Tex.) can be used in the methods described herein. See, e.g., Mullett et al. (2000) Methods 22: 77-91; Dong et al. (2002) Reviews in Mol Biotech 82: 303-323; Fivash et al. (1998) Curr Opin Biotechnol 9: 97-101; and Rich et al. (2000) Curr Opin Biotechnol 11:54-61.

The antibodies and fragments thereof can be, in some embodiments, “chimeric.” Chimeric antibodies and antigen-binding fragments thereof comprise portions from two or more different species (e.g., mouse and human). Chimeric antibodies can be produced with mouse variable regions of desired specificity spliced onto human constant domain gene segments (see, for example, U.S. Pat. No. 4,816,567). In this manner, non-human antibodies can be modified to make them more suitable for human clinical application (e.g., methods for treating or preventing a complement associated disorder in a human subject).

The monoclonal antibodies of the present disclosure include “humanized” forms of the non-human (e.g., mouse) antibodies. Humanized or CDR-grafted mAbs are particularly useful as therapeutic agents for humans because they are not cleared from the circulation as rapidly as mouse antibodies and do not typically provoke an adverse immune reaction. Methods of preparing humanized antibodies are generally well known in the art. For example, humanization can be essentially performed following the method of Winter and co-workers (see, e.g., Jones et al. (1986) Nature 321:522-525; Riechmann et al. (1988) Nature 332:323-327; and Verhoeyen et al. (1988) Science 239: 1534-1536), by substituting rodent CDRs or CDR sequences for the corresponding sequences of a human antibody. Also see, e.g., Staelens et al. (2006) Mol Immunol 43:1243-1257. In some embodiments, humanized forms of non-human (e.g., mouse) antibodies are human antibodies (recipient antibody) in which hypervariable (CDR) region residues of the recipient antibody are replaced by hypervariable region residues from a non-human species (donor antibody) such as a mouse, rat, rabbit, or non-human primate having the desired specificity, affinity, and binding capacity. In some instances, framework region residues of the human immunoglobulin are also replaced by corresponding non-human residues (so called “back mutations”). In addition, phage display libraries can be used to vary amino acids at chosen positions within the antibody sequence. The properties of a humanized antibody are also affected by the choice of the human framework. Furthermore, humanized and chimerized antibodies can be modified to comprise residues that are not found in the recipient antibody or in the donor antibody in order to further improve antibody properties, such as, for example, affinity or effector function.

Fully human antibodies are also provided in the disclosure. The term “human antibody” includes antibodies having variable and constant regions (if present) derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences (i.e., humanized antibodies). Fully human or human antibodies may be derived from transgenic mice carrying human antibody genes (carrying the variable (V), diversity (D), joining (J), and constant (C) exons) or from human cells. For example, it is now possible to produce transgenic animals (e.g., mice) that are capable, upon immunization, of producing a full repertoire of human antibodies in the absence of endogenous immunoglobulin production. (See, e.g., Jakobovits et al. (1993) Proc. Natl. Acad. Sci. USA 90:2551; Jakobovits et al. (1993) Nature 362:255-258; Bruggemann et al. (1993) Year in Immunol. 7:33; and Duchosal et al. (1992) Nature 355:258.) Transgenic mice strains can be engineered to contain gene sequences from unrearranged human immunoglobulin genes. The human sequences may code for both the heavy and light chains of human antibodies and would function correctly in the mice, undergoing rearrangement to provide a wide antibody repertoire similar to that in humans. The transgenic mice can be immunized with the target protein (to create a diverse array of specific antibodies and their encoding RNA. Nucleic acids encoding the antibody chain components of such antibodies may then be cloned from the animal into a display vector. Typically, separate populations of nucleic acids encoding heavy and light chain sequences are cloned, and the separate populations then recombined on insertion into the vector, such that any given copy of the vector receives a random combination of a heavy and a light chain. The vector is designed to express antibody chains so that they can be assembled and displayed on the outer surface of a display package containing the vector. For example, antibody chains can be expressed as fusion proteins with a phage coat protein from the outer surface of the phage. Thereafter, display packages can be screened for display of antibodies binding to a target.

Thus, in some embodiments, the disclosure provides, e.g., humanized, deimmunized or primatized antibodies comprising one or more of the complementarity determining regions (CDRs) of the mouse monoclonal antibodies described herein, which retain the ability (e.g., at least 50, 60, 70, 80, 90, or 100%, or even greater than 100%) of the mouse monoclonal antibody counterpart to bind to its antigen.

In addition, human antibodies can be derived from phage-display libraries (Hoogenboom et al. (1991) J. Mol. Biol. 227:381; Marks et al. (1991) J. Mol. Biol, 222:581-597; and Vaughan et al. (1996) Nature Biotech 14:309 (1996)). Synthetic phage libraries can be created which use randomized combinations of synthetic human antibody V-regions. By selection on antigen fully human antibodies can be made in which the V-regions are very human-like in nature. See, e.g., U.S. Pat. Nos. 6,794,132, 6,680,209, 4,634,666, and Ostberg et al. (1983), Hybridoma 2:361-367, the contents of each of which are incorporated herein by reference in their entirety.

For the generation of human antibodies, also see Mendez et al. (1998) Nature Genetics 15: 146-156 and Green and Jakobovits (1998) J. Exp. Med. 188:483-495, the disclosures of which are hereby incorporated by reference in their entirety. Human antibodies are further discussed and delineated in U.S. Pat. Nos. 5,939,598; 6,673,986; 6,114,598; 6,075,181; 6,162,963; 6,150,584; 6,713,610; and 6,657, 103 as well as U.S. Patent Application Publication Nos. 2003-0229905 A1, 2004-0010810 A1, US 2004-0093622 A1, 2006-0040363 A1, 2005-0054055 A1, 2005-0076395 A1, and 2005-0287630 A1. See also International Publication Nos. WO 94/02602, WO 96/34096, and WO 98/24893, and European Patent No. EP 0 463 151 B1. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in, e.g., U.S. Pat. Nos. 5,545,807; 5,545,806; 5,625,825; 5,625, 126; 5,633,425; 5,661,016; 5,770,429; 5,789,650; and U.S. Pat. Nos. 5,814,318; 5,591,669; 5,612,205; 5,721,367; 5,789,215; 5,643,763; 5,569,825; 5,877,397; 6,300,129; 5,874,299; 6,255,458; and 7,041,871, the disclosures of which are hereby incorporated by reference. See also European Patent No. 0 546 073 B1, International Patent Publication Nos. WO 92/03918, WO 92/22645, WO 92/22647, WO 92/22670, WO 93/12227, WO 94/00569, WO 94/25585, WO 96/14436, WO 97/13852, and WO 98/24884, the disclosures of each of which are hereby incorporated by reference in their entirety. See further Taylor et al. (1992) Nucleic Acids Res. 20: 6287; Chen et al. (1993) Int. Immunol. 5: 647; Tuaillon et al. (1993) Proc. Natl. Acad. Sci. USA 90: 3720-4; Choi et al. (1993) Nature Genetics 4: 1 17; Lonberg et al. (1994) Nature 368: 856-859; Taylor et al. (1994) International Immunology 6: 579-591; Tuaillon et al. (1995) J. Immunol. 154: 6453-65; Fishwild et al. (1996) Nature Biotechnology 14: 845; and Tuaillon et al. (2000) Eur. J. Immunol. 10: 2998-3005, the disclosures of each of which are hereby incorporated by reference in their entirety.

In some embodiments, de-immunized antibodies or antigen-binding fragments thereof are provided. De-immunized antibodies or antigen-binding fragments thereof are antibodies that have been modified so as to render the antibody or antigen-binding fragment thereof non-immunogenic, or less immunogenic, to a given species (e.g., to a human). De-immunization can be achieved by modifying the fusion proteins, antibodies or fragments thereof utilizing any of a variety of techniques known to those skilled in the art (see, e.g., PCT Publication Nos. WO 04/108158 and WO 00/34317). For example, fusion proteins, antibodies or fragments thereof may be de-immunized by identifying potential T cell epitopes and/or B cell epitopes within the amino acid sequence of the fusion proteins, antibodies or fragments thereof and removing one or more of the potential T cell epitopes and/or B cell epitopes from the fusion proteins, antibodies or fragments thereof, for example, using recombinant techniques. The modified antibody or antigen-binding fragment thereof may then optionally be produced and tested to identify antibodies or antigen-binding fragments thereof that have retained one or more desired biological activities, such as, for example, binding affinity, but have reduced immunogenicity. Methods for identifying potential T cell epitopes and/or B cell epitopes may be carried out using techniques known in the art, such as, for example, computational methods (see e.g., PCT Publication No. WO 02/069232), in vitro or in silico techniques, and biological assays or physical methods (such as, for example, determination of the binding of peptides to MHC molecules, determination of the binding of peptide:MHC complexes to the T cell receptors from the species to receive the fusion proteins, antibodies or fragments thereof, testing of the protein or peptide parts thereof using transgenic animals with the MHC molecules of the species to receive the antibody or antigen-binding fragment thereof, or testing with transgenic animals reconstituted with immune system cells from the species to receive the fusion proteins, antibodies or fragments thereof, etc.). In various embodiments, the de-immunized antibodies described herein include de-immunized antigen-binding fragments, Fab, Fv, scFv, Fab′ and F(ab′)₂, monoclonal antibodies, murine antibodies, engineered antibodies (such as, for example, chimeric, single chain, CDR-grafted, humanized, fully human antibodies, and artificially selected antibodies), synthetic antibodies and semi-synthetic antibodies.

In some embodiments, the present disclosure also provides bispecific antibodies. Bispecific antibodies are monoclonal, preferably human or humanized, antibodies that have binding specificities for at least two different antigens. For example, in one embodiment, a bispecific antibody of the invention comprises one domain with a binding specificity for p-selectin, and one domain with a binding specificity for a complement regulatory protein.

Methods for making bispecific antibodies are within the purview of those skilled in the art. Traditionally, the recombinant production of bispecific antibodies is based on the co-expression of two immunoglobulin heavy-chain/light-chain pairs, where the two heavy chain/light-chain pairs have different specificities (Milstein and Cuello (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, e.g., Suresh et al. (1986) Methods in Enzymology 121:210; PCT Publication No. WO 96/27011; Brennan et al. (1985) Science 229:81; Shalaby et al, J Exp Med (1992) 175:217-225; Kostelny et al. (1992) J Immunol 148(5): 1547-1553; Hollinger et al. (1993) Proc Natl Acad Sci USA 90:6444-6448; Gruber et al. (1994) J Immunol 152:5368; and Tutt et al. (1991) J Immunol 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5): 1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10): 1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-1g) molecules described in Wu et al. (2007) Nat Biotechnol 25(11): 1290-1297. The DVD-1g molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715.

The disclosure also provides camelid or dromedary antibodies (e.g., antibodies derived from Camelus bactrianus, Calelus dromaderius, or Lama paccos). Such antibodies, unlike the typical two-chain (fragment) or four-chain (whole antibody) antibodies from most mammals, generally lack light chains. See U.S. Pat. No. 5,759,808; Stijlemans et al. (2004) J Biol Chem 279: 1256-1261; Dumoulin et al. (2003) Nature 424:783-788; and Pleschberger et al. (2003) Bioconjugate Chem 14:440-448.

Engineered libraries of camelid antibodies and antibody fragments are commercially available, for example, from Ablynx (Ghent, Belgium). As with other antibodies of non-human origin, an amino acid sequence of a camelid antibody can be altered recombinantly to obtain a sequence that more closely resembles a human sequence, i.e., the nanobody can be “humanized” to thereby further reduce the potential immunogenicity of the antibody.

In some embodiments, the present disclosure also provides antibodies, or antigen-binding fragments thereof, which are variants of a peptide, protein or antibody described herein. In some embodiments, such a variant peptide, protein or antibody maintains the binding or inhibitory ability of the parent peptide, protein or antibody. Methods to prepare variants of known proteins, peptides or antibodies are known in the art. In some embodiments, such a variant comprises at least a single amino acid substitution, deletion, insertion, or other modification. In some embodiments, fusion proteins, antibodies or fragments thereof described herein comprises two or more (e.g. 2, 3, 4, 5, 6, 7, 8, 9, 10, 11, 12, 13, 14, 15, 16, 17, 18, 19, 20 or more) amino acid modifications (e.g., amino acid substitutions, deletions, or additions). In some embodiments, fusion proteins, antibodies or fragments thereof described herein does not contain an amino acid modification in a CDR. In some embodiments, fusion proteins, antibodies or fragments thereof described herein does contain one or more (e.g. 1, 2, 3, 4, 5, 6, 7, 8, 9, 10, 11, 12, 13, 14, 15, 16, 17, 18, 19, or 20) amino acid modifications in a CDR.

As used herein, the term “antibody fragment”, “antigen-binding fragment”, “antigen binding fragment”, or similar terms refer to fragment of an antibody that retains the ability to bind to an antigen wherein the antigen binding fragment may optionally include additional compositions not part of the original antibody (e.g. different framework regions or mutations) as well as the fragment(s) from the original antibody. Examples include, but are not limited to, a single chain antibody, a single chain Fv fragment (scFv), an Fd fragment, an Fab fragment, an Fab′ fragment, or an F(ab′)2 fragment. An scFv fragment is a single polypeptide chain that includes both the heavy and light chain variable regions of the antibody from which the scFv is derived. In addition, diabodies (Poljak (1994) Structure 2(12): 1121-1123; Hudson et al. (1999) J. Immunol. Methods 23(1-2): 177-189, the disclosures of each of which are incorporated herein by reference in their entirety), minibodies, triabodies (Schoonooghe et al. (2009) BMC Biotechnol 9:70), and domain antibodies (also known as “heavy chain immunoglobulins” or camelids; Holt et al. (2003) Trends Biotechnol 21(1 1):484-490), (the disclosures of each of which are incorporated herein by reference in their entirety) that bind to a complement component protein can be incorporated into the compositions, and used in the methods, described herein. In some embodiments, any of the antigen binding fragments described herein may be included under “antigen binding fragment thereof or equivalent terms, when referring to fragments related to an antibody, whether such fragments were actually derived from the antibody or are antigen binding fragments that bind the same epitope or an overlapping epitope or an epitope contained in the antibody's epitope. An antigen binding fragment thereof may include antigen-binding fragments that bind the same, or overlapping, antigen as the original antibody and wherein the antigen binding fragment includes a portion (e.g. one or more CDRs, one or more variable regions, etc.) that is a fragment of the original antibody.

In some embodiments, the antibodies described herein comprise an altered or mutated sequence that leads to altered stability or half-life compared to parent antibodies. This includes, for example, an increased stability or half-life for higher affinity or longer clearance time in vitro or in vivo, or a decreased stability or half-life for lower affinity or quicker removal. Additionally, the antibodies described herein may contain one or more (e.g. 1, 2, 3, 4, 5, 6, 7, 8, 9, 10, 11, 12, 13, 14, 15, 16, 17, 18, 19, or 20) amino acid substitutions, deletions, or insertions that result in altered post-translational modifications, including, for example, an altered glycosylation pattern (e.g., the addition of one or more sugar components, the loss of one or more sugar components, or a change in composition of one or more sugar components.

In some embodiments, the antibodies described herein comprise reduced (e.g. or no) effector function. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

Antibodies with altered or no effector functions may be generated by engineering or producing antibodies with variant constant, Fc, or heavy chain regions; recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g., glycosylation patterns, may be achieved by manipulating the cell culture and expression conditions by which the antibody is produced. Suitable methods for introducing one or more substitutions, additions, or deletions into an Fc region of an antibody are well known in the art and include, e.g., standard DNA mutagenesis techniques as described in, e.g., Sambrook et al. (1989) “Molecular Cloning: A Laboratory Manual, 2nd Edition,” Cold Spring Harbor Laboratory Press, Cold Spring Harbor, N.Y.; Harlow and Lane (1988), supra; Borrebaek (1992), supra; Johne et al. (1993), supra; PCT publication no. WO 06/53301; and U.S. Pat. No. 7,704,497.

### Nucleic Acid Molecules

Provided herein are polynucleotides that encode the antibodies or fragments thereof of the invention. Such polynucleotide may also be used for delivery and expression of molecule. For example, in some embodiments, there is provided a polynucleotide encoding a fusion protein comprising a p-selectin binding or blocking domain, or a fragment thereof, as described herein, operably linked to cargo domain comprising a nucleotide sequence encoding a cargo molecule, as described herein. In some embodiments, the cargo molecule is a protein, a peptide, a nucleic acid molecule, an antibody or an antibody fragment. In some embodiments, the cargo molecule is a therapeutic molecule for the treatment of a disease or disorder. In some embodiments, the cargo domain comprises a complement inhibitor. In some embodiments, the polynucleotide also comprises a sequence encoding a signal peptide operably linked at the 5′ end of the encoding sequence. In some embodiments, the polynucleotide also comprises a sequence encoding a linker sequence.

In one embodiment, the nucleic acid molecule comprises a nucleotide sequence that encodes a non-blocking p-selectin antibody comprising at least one of a heavy chain (HC) CDR1 sequence comprising SEQ ID NO:13, a HC CDR2 sequence comprising SEQ ID NO:15, a HC CDR3 sequence comprising SEQ ID NO:17, a light chain (LC) CDR1 sequence comprising SEQ ID NO:19, a LC CDR2 sequence comprising SEQ ID NO:21, and a LC CDR3 sequence comprising SEQ ID NO:23. In one embodiment, the nucleotide sequence encodes a non-blocking p-selectin antibody comprising 1, 2, 3, 4, 5, or all 6 CDRs as set forth in SEQ ID NO:13, SEQ ID NO:15, SEQ ID NO:17, SEQ ID NO:19, SEQ ID NO:21 and SEQ ID NO:23. In one embodiment, the nucleic acid molecule comprises at least one of a nucleotide sequence comprising SEQ ID NO:14 encoding a HC CDR1, a nucleotide sequence comprising SEQ ID NO:16 encoding a HC CDR2, a nucleotide sequence comprising SEQ ID NO:18 encoding a HC CDR3, a nucleotide sequence comprising SEQ ID NO:20 encoding a LC CDR1, a nucleotide sequence comprising SEQ ID NO:22 encoding a LC CDR2, and a nucleotide sequence comprising SEQ ID NO:24 encoding a LC CDR3. In one embodiment, the nucleotide sequence comprises nucleotide sequences encoding 1, 2, 3, 4, 5, or all 6 CDRs as set forth in SEQ ID NO:14, SEQ ID NO:16, SEQ ID NO:18, SEQ ID NO:20, SEQ ID NO:22 and SEQ ID NO:24.

In one embodiment, the nucleic acid molecule comprises a nucleotide sequence that encodes a non-blocking p-selectin antibody comprising at least one of a HC CDR1 sequence comprising SEQ ID NO:13, a HC CDR2 sequence comprising SEQ ID NO:15, a HC CDR3 sequence comprising SEQ ID NO:17, a LC CDR1 sequence comprising SEQ ID NO:28, a LC CDR2 sequence comprising SEQ ID NO:30, and a LC CDR3 sequence comprising SEQ ID NO:32. In one embodiment, the nucleotide sequence encodes a non-blocking p-selectin antibody comprising 1, 2, 3, 4, 5, or all 6 CDRs as set forth in SEQ ID NO:13, SEQ ID NO:15, SEQ ID NO:17, SEQ ID NO:28, SEQ ID NO:30 and SEQ ID NO:32. In one embodiment, the nucleic acid molecule comprises at least one of a nucleotide sequence comprising SEQ ID NO:25 encoding a HC CDR1, a nucleotide sequence comprising SEQ ID NO:26 encoding a HC CDR2, a nucleotide sequence comprising SEQ ID NO:27 encoding a HC CDR3, a nucleotide sequence comprising SEQ ID NO:29 encoding a LC CDR1, a nucleotide sequence comprising SEQ ID NO:31 encoding a LC CDR2, and a nucleotide sequence comprising SEQ ID NO:33 encoding a LC CDR3. In one embodiment, the nucleotide sequence comprises nucleotide sequences encoding 1, 2, 3, 4, 5, or all 6 CDRs as set forth in SEQ ID NO:25, SEQ ID NO:26, SEQ ID NO:27, SEQ ID NO:29, SEQ ID NO:31 and SEQ ID NO:33.

In one embodiment, the nucleic acid molecule comprises a nucleotide sequence that encodes a blocking p-selectin antibody comprising at least one of a HC CDR1 sequence comprising SEQ ID NO:34, a HC CDR2 sequence comprising SEQ ID NO:36, a HC CDR3 sequence comprising SEQ ID NO:38, a LC CDR1 sequence comprising SEQ ID NO:40, a LC CDR2 sequence comprising SEQ ID NO:42, and a LC CDR3 sequence comprising SEQ ID NO:44. In one embodiment, the nucleotide sequence encodes a non-blocking p-selectin antibody comprising 1, 2, 3, 4, 5, or all 6 CDRs as set forth in SEQ ID NO:34, SEQ ID NO:36, SEQ ID NO:38, SEQ ID NO:40, SEQ ID NO:42 and SEQ ID NO:44. In one embodiment, the nucleic acid molecule comprises at least one of a nucleotide sequence comprising SEQ ID NO:35 encoding a HC CDR1, a nucleotide sequence comprising SEQ ID NO:37 encoding a HC CDR2, a nucleotide sequence comprising SEQ ID NO:39 encoding a HC CDR3, a nucleotide sequence comprising SEQ ID NO:41 encoding a LC CDR1, a nucleotide sequence comprising SEQ ID NO:43 encoding a LC CDR2, and a nucleotide sequence comprising SEQ ID NO:45 encoding a LC CDR3. In one embodiment, the nucleotide sequence comprises nucleotide sequences encoding 1, 2, 3, 4, 5, or all 6 CDRs as set forth in SEQ ID NO:35, SEQ ID NO:37, SEQ ID NO:39, SEQ ID NO:41, SEQ ID NO:43 and SEQ ID NO:45.

In some embodiments, the nucleotide sequence encodes a non-blocking anti-p-selectin antibody, or fragment thereof. In some embodiments, the nucleotide sequence encodes an amino acid sequence as set forth in SEQ ID NO:2, or SEQ ID NO:6, or a fragment or variant thereof. In some embodiments, the nucleotide sequence comprises SEQ ID NO:1 or SEQ ID NO:5 or a fragment or variant thereof.

In some embodiments, the nucleotide sequence encodes a blocking anti-p-selectin antibody, or fragment thereof. In some embodiments, the nucleotide sequence encodes an amino acid sequence as set forth in SEQ ID NO:10, or a fragment or variant thereof. In some embodiments, the nucleotide sequence comprises SEQ ID NO:9, or a fragment or variant thereof.

In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined nucleotide sequence. In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of a nucleotide sequence of SEQ ID NO:1, SEQ ID NO:5 or SEQ ID NO:9.

In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of a defined nucleotide sequence. In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of SEQ ID NO:1, SEQ ID NO:5 or SEQ ID NO:9.

Also provided are expression vectors comprising a polynucleotide described herein for expression of the proteins, peptides, antibodies, antibody fragments or fusion proteins of the invention. The expression vector can be used to direct expression of proteins, peptides, antibodies, antibody fragments or fusion proteins in vitro or in vivo. The vector may include any element to establish a conventional function of a vector, for example, promoter, terminator, selection marker, and origin of replication. The promoter can be constitutive or regulative, and is selected from, for example, promoters of genes for galactokinase (GAL1), uridylyltransferase (GALT), epimerase (GAL10), phosphoglycerate kinase (PGK), glyceraldehydes-3-phosphate dehydrogenase (GPD), alcohol dehydrogenase (ADH), and the like.

Many expression vectors are known to those of skill in the art. For example, E. coli may be transformed using pBR322, a plasmid derived from an E. coli species (Mandel et al., J. Mol. Biol., 53:154 (1970)). Plasmid pBR322 contains genes for ampicillin and tetracycline resistance, and thus provides easy means for selection. Other vectors include different features such as different promoters, which are often important in expression. For example, plasmids pKK223-3 (Pharmacia Fine Chemicals, Uppsala, Sweden), pKK233-2 (Clontech, Palo Alto, Calif., USA), and pGEM1 (Promega Biotech, Madison, Wis., USA), are all commercially available. Other vectors that can be used in the present invention include, but are not limited to, pET21a (Studier et al., Methods Enzymol., 185: 60-89 (1990)), pR1T5, and pR1T2T (Pharmacia Biotechnology), and pB0475 (Cunningham et al., Science, 243: 1330-1336 (1989); U.S. Pat. No. 5,580,723). Mammalian expression vectors may contain non-transcribed elements such as an origin of replication, promoter and enhancer, and 5′ or 3′ nontranslated sequences such as ribosome binding sites, a polyadenylation site, acceptor site and splice donor, and transcriptional termination sequences. Promoters for use in mammalian expression vectors usually are for example viral promoters such as Polyoma, Adenovirus, HTLV, Simian Virus 40 (SV 40), and human cytomegalovirus (CMV). Vectors can also be constructed using standard techniques by combining the relevant traits of the vectors described above.

Also provided are host cells (such as isolated cells, transient cell lines, and stable cell lines) for expressing the molecule described herein. The host cell may be prokaryotic or eukaryotes. Exemplary prokaryote host cells include E. coli K12 strain 294 (ATCC No. 31446), E. coli B, E. coli X1776 (ATCC No. 31537), E. coli W3110 (F-, gamma-, prototrophic/ATCC No. 27325), bacilli such as Bacillus subtilis, and other enterobacteriaceae such as Salmonella typhimurium or Serratia marcesans, and various Pseudomonas species. One suitable prokaryotic host cell is E. coli BL21 (Stratagene), which is deficient in the OmpT and Lon proteases, which may interfere with isolation of intact recombinant proteins, and useful with T7 promoter-driven vectors, such as the pET vectors. Another suitable prokaryote is E. coli W3110 (ATCC No. 27325). When expressed by prokaryotes the peptides typically contain an N-terminal methionine or a formyl methionine and are not glycosylated. In the case of fusion proteins, the N-terminal methionine or formyl methionine resides on the amino terminus of the fusion protein or the signal sequence of the fusion protein. These examples are, of course, intended to be illustrative rather than limiting.

In addition to prokaryotes, eukaryotic microbes such as filamentous fungi or yeast are suitable cloning or expression hosts for fusion-protein-encoding vectors. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC No. 16,045), K. wickeramii (ATCC No. 24,178), K. waltii (ATCC No. 56,500), K. drosophilarum (ATCC No. 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc. Natl. Acad. Sci. USA, 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc. Natl. Acad. Sci. USA, 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982). Host cells also include insect cells such as Drosophila S2 and Spodoptera Sf9, as well as plant cells.

Examples of useful mammalian host cell lines include, but are not limited to, HeLa, Chinese hamster ovary (CHO), COS-7, L cells, C127, 3T3, BHK, CHL-1, NSO, HEK293, W138, BHK, C127 or MDCK cell lines. Another exemplary mammalian cell line is CHL-1. When CHL-1 is used hygromycin is included as a eukaryotic selection marker. CHL-1 cells are derived from RPMI 7032 melanoma cells, a readily available human cell line. Cells suitable for use in this invention are commercially available from the ATCC.

In some embodiments, the host cell is a non-human host cell. In some embodiment, the host cell is a CHO cell. In some embodiments, the host cell is a 293 cell.

The molecules can be isolated by a variety of methods known in the art. In some embodiments, when the molecule is a fusion protein secreted into the growth media, the molecule can be purified directly from the media. If the fusion protein is not secreted, it is isolated from cell lysates. Cell disruption can be done by any conventional method, including freeze-thaw cycling, sonication, mechanical disruption, or use of cell lysing agents. The molecules can be obtained by various methods. These include, but are not limited to, immunoaffinity chromatography, reverse phase chromatography, cation exchange chromatography, anion exchange chromatography, hydrophobic interaction chromatography, gel filtration chromatography, and HPLC. For example, the molecule can be purified by immunoaffinity chromatography using an antibody that recognizes the targeting portion or an antibody that recognizes the inhibitor portion, or both. In some embodiments, the molecule is purified by ion change chromatography.

The peptide may or may not be properly folded when expressed as a fusion protein. These factors determine whether the fusion protein must be denatured and refolded, and if so, whether these procedures are employed before or after cleavage. When denaturing and refolding are needed, typically the peptide is treated with a chaotrope, such a guanidine HCl, and is then treated with a redox buffer, containing, for example, reduced and oxidized dithiothreitol or glutathione at the appropriate ratios, pH, and temperature, such that the peptide is refolded to its native structure.

### Fusion Molecules

In some embodiments, the invention provides a fusion molecule comprising a domain that specifically binds to p-selectin fused to a cargo domain. In one embodiment, the cargo domain comprises a protein, peptide, nucleic acid molecule, small molecule, antibody or antibody fragment for activating or inhibiting a protein or pathway. In one embodiment, the cargo domain comprises a therapeutic agent for the treatment of a disease or disorder.

In one embodiment, the invention provides a fusion molecule comprising a domain that specifically binds to p-selectin fused to a complement inhibitor domain. In some embodiments, the invention provides a fusion molecule comprising a domain that blocks p-selectin fused to a complement inhibitor domain.

A “fusion protein” as used herein refers to two or more peptides, polypeptides, or proteins operably linked to each other. In some embodiments, the p-selectin binding or blocking domain and the complement inhibitory domain are directly fused to each other. In some embodiments, the targeting portion and inhibitor portion are linked by an amino acid linker sequence. Examples of linker sequences include, but are not limited to, (Gly4Ser), (Gly4Ser)₂, (Gly4Ser)₃, (Gly3Ser)₄, (SerGly4), (SerGly4)₂, (SerGly4)₃, and (SerGly4)₄. The order of the p-selectin binding or blocking domain and the complement inhibitory domain in the fusion protein can vary. For example, in some embodiments, the C-terminus of the p-selectin binding or blocking domain is fused (directly or indirectly) to the N-terminus of the complement inhibitory domain. In some embodiments, the N-terminus of the p-selectin binding or blocking domain is fused (directly or indirectly) to the C-terminus of the complement inhibitory domain.

In some embodiments, the p-selectin binding or blocking domain and the complement inhibitory domain are linked via a chemical cross-linker. Linking of the two domains can occur on reactive groups located on the two moieties. Reactive groups that can be targeted using a crosslinker include primary amines, sulfhydryls, carbonyls, carbohydrates, and carboxylic acids, or active groups that can be added to proteins. Examples of chemical linkers are well known in the art and include, but are not limited to, bismaleimidohexane, maleimidobenzoyl-N-hydroxysuccinimide ester, NHS-Esters-Maleimide Crosslinkers such as SPDP, carbodiimide, glutaraldehyde, MBS, Sulfo-MBS, SMPB, sulfo-SMPB, GMBS, Sulfo-GMBS, EMCS, Sulfo-EMCS, imidoester crosslinkers such as DMA, DMP, DMS, DTBP, EDC and DTME.

In some embodiments, the p-selectin binding or blocking domain and the complement inhibitory domain are non-covalently linked. For example, the two domains may be brought together by two interacting bridging proteins (such as biotin and streptavidin), each linked to the p-selectin binding or blocking domain or the complement inhibitory domain.

In one embodiment, the fusion molecule of the invention comprises at least one antibody specific for binding to p-selectin. In one embodiment, the anti-p-selectin antibody of the invention binds to p-selectin, but does not alter the activity of p-selectin. In such an embodiment the anti-p-selectin antibody is a non-blocking antibody. In one embodiment, the fusion molecule of the invention comprises at least one antibody specific for binding to p-selectin which also inhibits the activity of p-selectin. In such an embodiment the anti-p-selectin antibody is a blocking antibody. In one embodiment, the p-selectin binding domain comprises a non-blocking p-selectin antibody comprising a sequence as set forth in SEQ ID NO:2 or SEQ ID NO:6, or a fragment or variant thereof. In one embodiment, the p-selectin binding domain comprises a p-selectin blocking antibody comprising a sequence as set forth in SEQ ID NO:10, or a fragment or variant thereof.

In some embodiments, the invention provides a nucleotide sequence encoding a fusion molecule of the invention comprising a p-selectin binding domain. In one embodiment, the nucleotide sequence encoding the p-selectin binding domain encodes a non-blocking p-selectin antibody comprising a sequence as set forth in SEQ ID NO:2 or SEQ ID NO:6, or a fragment or variant thereof. In one embodiment, the nucleotide sequence comprises a sequence as set forth in SEQ ID NO:1 or SEQ ID NO:5, or a fragment or variant thereof. In one embodiment, the nucleotide sequence encoding the p-selectin binding domain encodes a blocking p-selectin antibody comprising a sequence as set forth in SEQ ID NO:10, or a fragment or variant thereof. In one embodiment, the nucleotide sequence comprises a sequence as set forth in SEQ ID NO:9, or a fragment or variant thereof.

In some embodiments, an anti-p-selectin complement inhibitory fusion molecule of the invention comprises a blocking or non-blocking anti-p-selectin antibody, or fragment thereof, fused to an inhibitor of a complement protein or complement signaling. In one embodiment, the fusion molecule comprises a non-blocking anti-p-selectin antibody or fragment thereof fused to an inhibitor of the classical, alternative or lectin pathway, including, but not limited to, inhibitors of C1, manna binding lectin protease, C3 convertase, C5 convertase, the membrane attack complex. In one embodiment, the inhibitor of a complement protein or complement signaling is a protein, a peptide, a nucleic acid molecule, a small molecule, an antibody, or an antibody fragment. Exemplary inhibitors of complement include, but are not limited to, Factor H (FH), Decay Accelerating Factor (DAF or CD55), Membrane Cofactor Protein (MCP or CD46), Protectin (CD59), Crry (murine equivalent of MCP), Mannose-binding lectin-associated protein of 44 kDa (MAp44), Complement C3b/C4b Receptor 1 (CR1 or CD35), Complement Regulator of the Immunoglobulin Superfamily (CRIg), C4-Binding Protein (C4 bp), OMS721, Eculizumab, Ravulizumab, Coversin, CCX168, IFX 1, CCX168, AMY-101, APL-2, ACH 4471, LPN023, Cemdisiran, C1INH, LFG-316, and plasma serine proteinase inhibitor serpin or fragments thereof.

In some embodiments, the anti-p-selectin complement inhibitory fusion molecule of the invention comprises an anti-p-selectin antibody, or fragment thereof, fused to CR1 or a fragment thereof, factor H or a fragment thereof, DAF or a fragment thereof, C4 bp or a fragment thereof, Map44 or a fragment thereof, sMAP or a fragment thereof, CD59 or a fragment thereof, CRIg or a fragments thereof, or an antibody or fragment thereof that recognizes a complement protein or complement activation product.

In some embodiments, the anti-p-selectin complement inhibitory fusion molecule of the invention comprises a non-blocking anti-p-selectin antibody, or fragment thereof, fused to Crry. In some embodiments, the anti-p-selectin complement inhibitory fusion molecule of the invention comprises an amino acid sequence as set forth in SEQ ID NO:4 or SEQ ID NO:8, or a fragment or variant thereof.

In some embodiments, the anti-p-selectin complement inhibitory fusion molecule of the invention comprises a blocking anti-p-selectin antibody, or fragment thereof, fused to Crry. In some embodiments, the anti-p-selectin complement inhibitory fusion molecule of the invention comprises an amino acid sequence as set forth in SEQ ID NO:12, or a fragment or variant thereof.

In some embodiments, the anti-p-selectin complement inhibitory fusion molecule of the invention comprises a non-blocking anti-p-selectin antibody, or fragment thereof, fused to CR1. In some embodiments, the anti-p-selectin complement inhibitory fusion molecule of the invention comprises an amino acid sequence as set forth in SEQ ID NO:51 or SEQ ID NO:53, or a fragment or variant thereof.

In some embodiments, the anti-p-selectin complement inhibitory fusion molecule of the invention comprises a blocking anti-p-selectin antibody, or fragment thereof, fused to CR1. In some embodiments, the anti-p-selectin complement inhibitory fusion molecule of the invention comprises an amino acid sequence as set forth in SEQ ID NO:47 or SEQ ID NO:49, or a fragment or variant thereof.

In some embodiments, the invention provides a nucleotide sequence encoding an anti-p-selectin complement inhibitory fusion molecule of the invention, or a fragment thereof. In some embodiments, the nucleotide sequence encodes a blocking or non-blocking anti-p-selectin antibody, or fragment thereof, fused to an inhibitor of a complement protein or complement signaling. In one embodiment, the fusion molecule comprises a non-blocking anti-p-selectin antibody or fragment thereof fused to an inhibitor of the classical, alternative or lectin pathway, including, but not limited to, inhibitors of C1, manna binding lectin protease, C3 convertase, C5 convertase, the membrane attack complex. In one embodiment, the inhibitor of a complement protein or complement signaling is a protein, a peptide, a nucleic acid molecule, a small molecule, an antibody, or an antibody fragment.

In some embodiments, the nucleotide sequence encodes a non-blocking anti-p-selectin antibody, or fragment thereof, fused to Crry. In some embodiments, the nucleotide sequence encodes an amino acid sequence as set forth in SEQ ID NO:4, or SEQ ID NO:8, or a fragment or variant thereof. In some embodiments, the nucleotide sequence comprises SEQ ID NO:3, or SEQ ID NO:7, or a fragment or variant thereof.

In some embodiments, the nucleotide sequence encodes a blocking anti-p-selectin antibody, or fragment thereof, fused to Crry. In some embodiments, the nucleotide sequence encodes an amino acid sequence as set forth in SEQ ID NO:12, or a fragment or variant thereof. In some embodiments, the nucleotide sequence comprises SEQ ID NO:11, or a fragment or variant thereof.

In some embodiments, the nucleotide sequence encodes a non-blocking anti-p-selectin antibody, or fragment thereof, fused to CR1. In some embodiments, the nucleotide sequence encodes an amino acid sequence as set forth in SEQ ID NO:51, or SEQ ID NO:53, or a fragment or variant thereof. In some embodiments, the nucleotide sequence comprises SEQ ID NO:50, or SEQ ID NO:52, or a fragment or variant thereof.

In some embodiments, the nucleotide sequence encodes a blocking anti-p-selectin antibody, or fragment thereof, fused to CR1. In some embodiments, the nucleotide sequence encodes an amino acid sequence of SEQ ID NO:47 or SEQ ID NO:49, or a fragment or variant thereof. In some embodiments, the nucleotide sequence comprises SEQ ID NO:46 or SEQ ID NO:48, or a fragment or variant thereof.

In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined amino acid sequence. In some embodiments, a variant of an amino acid sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of an amino acid sequence of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:53. In some embodiments, the variant of the amino acid sequence comprising at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or higher identity over the full length of an amino acid sequence of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:5 comprises 100% identity to all three CDR sequences of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:53.

In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared to a defined nucleotide sequence. In some embodiments, a variant of a nucleotide sequence as described herein comprises at least about 60% identity, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over the full length of a nucleotide sequence of SEQ ID NO:1, SEQ ID NO:3, SEQ ID NO:5, SEQ ID NO:7, SEQ ID NO:9, SEQ ID NO:11, SEQ ID NO:46, SEQ ID NO:48, SEQ ID NO:50 or SEQ ID NO:52. In some embodiments, the variant of the nucleotide sequence comprising at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or higher identity over the full length of a nucleotide sequence of SEQ ID NO:1, SEQ ID NO:3, SEQ ID NO:5, SEQ ID NO:7, SEQ ID NO:9, SEQ ID NO:11, SEQ ID NO:46, SEQ ID NO:48, SEQ ID NO:50 or SEQ ID NO:52 comprises 100% identity to all three CDR sequences of SEQ ID NO:1, SEQ ID NO:3, SEQ ID NO:5, SEQ ID NO:7, SEQ ID NO:9, SEQ ID NO:11, SEQ ID NO:46, SEQ ID NO:48, SEQ ID NO:50 or SEQ ID NO:52.

In some embodiments, a fragment of an amino acid sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of a defined amino acid sequence. In some embodiments, a fragment of an amino acid sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% 94%, 95%, 96%, 97%, 98%, or 99% of the full length sequence of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:53. In some embodiments, the fragment of the amino acid sequence comprising at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:53 comprises all three CDR sequences of SEQ ID NO:2, SEQ ID NO:4, SEQ ID NO:6, SEQ ID NO:8, SEQ ID NO:10, SEQ ID NO:12, SEQ ID NO:47, SEQ ID NO:49, SEQ ID NO:51 or SEQ ID NO:53.

In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of a defined nucleotide sequence. In some embodiments, a fragment of a nucleotide sequence as described herein comprises at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of SEQ ID NO:1, SEQ ID NO:3, SEQ ID NO:5, SEQ ID NO:7, SEQ ID NO:9, SEQ ID NO:11, SEQ ID NO:46, SEQ ID NO:48, SEQ ID NO:50 or SEQ ID NO:52. In some embodiments, the fragment of the nucleotide sequence comprising at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full length sequence of SEQ ID NO:1, SEQ ID NO:3, SEQ ID NO:5, SEQ ID NO:7, SEQ ID NO:9, SEQ ID NO: 11, SEQ ID NO:46, SEQ ID NO:48, SEQ ID NO:50 or SEQ ID NO:52 comprises all three CDR sequences of SEQ ID NO:1, SEQ ID NO:3, SEQ ID NO:5, SEQ ID NO:7, SEQ ID NO:9, SEQ ID NO:11, SEQ ID NO:46, SEQ ID NO:48, SEQ ID NO:50 or SEQ ID NO:52.

### Detectable Moieties

The molecules described herein may also contain a tag or detectable moiety. This tag or detectable moiety can be fused to the C-terminus or N-terminus of the protein, peptide, antibody, antibody fragment, or fusion molecule of the invention. In some embodiments, the tag or detectable moiety can be used to facilitate protein purification. In some embodiments, the tag or detectable moiety allows for visualizatoin of the molecule using various imaging modalities.

For example, in some embodiments, MRI can be used to non-invasively acquire tissue images with high resolution. Paramagnetic agents or nanoparticles or aggregates thereof enhance signal attenuation on T2-weighted magnetic resonance images, and conjugation of such nanoparticles to binding ligands (e.g., the protein, peptide, antibody, antibody fragment, or fusion molecule of the invention) permits the detection of specific molecules at the cellular level. For example, MRI with nanoparticle detection agents can image cell migration (J. W. Bulte et al, 2001, Nat. Biotechnol. 19: 1141-1147), apoptosis (M. Zhao et al., 2001, Nat. Med. 7: 1241-1244), and can detect small foci of cancer. See e.g., Y. W. Jun et al, 2005, J. Am. Chem. Soc. 127:5732-5733; Y. M. Huh et al, 2005, J. Am. Chem. Soc. 127: 12387-12391. Contrast-enhanced MRI is well-suited for the dynamic non-invasive imaging of macromolecules or of molecular events, but it requires ligands that specifically bind to the molecule of interest. J. W. Bulte et al, 2004, NMR Biomed. 17:484-499. Fluorescent dyes and fluorophores (e.g. fluorescein, fluorescein isothiocyanate, and fluorescein derivatives) can be used to non-invasively acquire tissue images with high resolution, with for example spectrophotometry, two-photon fluorescence, two-photon laser microscropy, or fluorescence microscopy (e.g. of tissue biopsies). MRI can be used to non-invasively acquire tissue images with high resolution, with for example paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, other nanoparticle contrast agents. MRI can be used to non-invasively acquire tissue images with high resolution, with for example Gadolinium, including liposomes or other delivery vehicles containing Gadolinium chelate (“Gd-chelate”) molecules. Positron emission tomography (PET), PET/computed tomography (CT), single photon emission computed tomography (SPECT), and SPECT/CT can be used to non-invasively acquire tissue images with high resolution, with for example radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia. Ultrasound (ultrasonography) and contrast enhanced ultrasound (contrast enhanced ultrasonography) can be used to non-invasively acquire tissue images with high resolution, with for example biocolloids or microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.). X-ray imaging (radiography) or CT can be used to non-invasively acquire tissue images with high resolution, with for example iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, or gold nanoparticle aggregates. These detectable moieties capable of being measured or detected by the corresponding method are non-limiting examples of detectable moieties that can be included in or conjugated to a protein, peptide, antibody, antibody fragment, or fusion molecule of the invention.

### Pharmaceutical Compositions

In some embodiments, the present disclosure provides a pharmaceutical composition including any of the isolated fusion proteins, antibodies or fragments thereof described in this disclosure. In some embodiments, the present disclosure provides a pharmaceutical composition including a nucleic acid encoding fusion proteins, antibodies or fragments thereof described in this disclosure. In some embodiments, the present disclosure provides a pharmaceutical composition including a vector containing the nucleic acid sequence of an isolated nucleic acid encoding the fusion proteins, antibodies or fragments thereof described in this disclosure. In some embodiments, the present disclosure provides a pharmaceutical composition including a cell containing such vector described herein.

In some embodiments, the present disclosure provides a pharmaceutical composition including any of the fusion proteins, antibodies or fragments thereof, or nucleic acid molecules encoding the same, described in this disclosure and a therapeutically acceptable excipient. Suitable excipients are well known in the art and recited herein.

In another embodiment, provided herein are articles of manufacture or kits containing diagnostic compositions including an effective amount of any of the fusion proteins, antibodies or fragments thereof, or nucleic acid molecules encoding the same, and instructions for their use in the methods described herein. The diagnostic compositions may further include one or more pharmaceutically acceptable excipients formulated for administration to an individual as described herein. The kit may further include means for administration, such as a syringe, inhaler or other device useful for systemic administration or local administration.

In yet another embodiment, the disclosure features an article of manufacture including: a container including a label; and a composition including any of the fusion proteins, antibodies or fragments thereof, or nucleic acid molecules encoding the same, described herein, wherein the label indicates that the composition is to be administered to a human having, suspected of having, or at risk for developing, a complement-associated disorder, disease, or condition. The article of manufacture can include one or more additional agents.

In another aspect, the disclosure features a diagnostic or monitoring kit including: (i) any of the fusion proteins, antibodies or fragments thereof, or nucleic acid molecules encoding the same, described herein and (ii) means for delivering the fusion proteins, antibodies or fragments thereof, or nucleic acid molecules encoding the same, to a human; or (ii) any of the constructs described herein and (iv) means for delivering the construct to a human. The means can be suitable for subcutaneous delivery of the construct to the human. The means can be suitable for intraocular delivery of the construct, or the fusion proteins, antibodies or fragments thereof, to the human. The means can be suitable for intraarticular delivery of the construct, or the fusion proteins, antibodies or fragments thereof, to the human.

The pharmaceutical compositions may be suitable for a variety of modes of administration described herein, including for example systemic or localized administration. The pharmaceutical compositions can be in the form of eye drops, injectable solutions, or in a form suitable for inhalation (either through the mouth or the nose) or oral administration. The pharmaceutical compositions described herein can be packaged in single unit dosages or in multidosage forms.

In some embodiments, the pharmaceutical compositions comprise a pharmaceutically acceptable carrier suitable for administration to human. In some embodiments, the pharmaceutical compositions comprise a pharmaceutically acceptable carrier suitable for intraocular injection. In some embodiments, the pharmaceutical compositions comprise a pharmaceutically acceptable carrier suitable for topical application. In some embodiments, the pharmaceutical compositions comprise a pharmaceutically acceptable carrier suitable for intravenous injection. In some embodiments, the pharmaceutical compositions comprise and a pharmaceutically acceptable carrier suitable for injection into the arteries.

The compositions are generally formulated as sterile, substantially isotonic, and in full compliance with all Good Manufacturing Practice (GMP) regulations of the U.S. Food and Drug Administration. In some embodiments, the composition is free of pathogen. For injection, the pharmaceutical composition can be in the form of liquid solutions, for example in physiologically compatible buffers such as Hank's solution or Ringer's solution. In addition, the pharmaceutical composition can be in a solid form and redissolved or suspended immediately prior to use. Lyophilized compositions are also included.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

The present invention in some embodiments provides compositions comprising a targeted molecule and a pharmaceutically acceptable carrier suitable for administration to the eye. Such pharmaceutical carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium state, glycerol monostearate, glycerol, propylene, water, and the like. The pharmaceutical composition, if desired, can also contain minor amounts of wetting or emulsifying agents, or pH buffering agents. The molecule and other components of the composition may be encased in polymers or fibrin glues to provide controlled release of the molecule. These compositions can take the form of solutions, suspensions, emulsions, ointment, gel, or other solid or semisolid compositions, and the like. The compositions typically have a pH in the range of 4.5 to 8.0. The compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

In some embodiment, the composition is formulated in accordance with routine procedures as a pharmaceutical composition adapted for injection intravenously, intraperitoneally, or intracranially. Typically, compositions for injection are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions may further comprise additional ingredients, for example preservatives, buffers, tonicity agents, antioxidants and stabilizers, nonionic wetting or clarifying agents, viscosity-increasing agents, and the like.

Suitable preservatives for use in a solution include polyquaternium-1, benzalkonium chloride, thimerosal, chlorobutanol, methyl paraben, propyl paraben, phenylethyl alcohol, edetate disodium, sorbic acid, benzethonium chloride, and the like. Typically (but not necessarily), such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The use of viscosity enhancing agents to provide topical compositions with viscosities greater than the viscosity of simple aqueous solutions may be desirable. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

In some embodiments, there is provided a pharmaceutical composition for delivery of a nucleotide encoding the molecule. The pharmaceutical composition for gene therapy can be in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical composition can comprise one or more cells which produce the gene delivery system.

In clinical settings, a gene delivery system for a gene therapeutic can be introduced into a subject by any of a number of methods. For instance, a pharmaceutical composition of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter, See U.S. Pat. No. 5,328,470, or by stereotactic injection, Chen et al. (1994), Proc. Natl. Acad. Sci., USA 91: 3054-3057. A polynucleotide encoding a targeted inhibitor molecule can be delivered in a gene therapy construct by electroporation using techniques described, Dev et al. (1994), Cancer Treat. Rev. 20:105-115.

### Dosing

The optimal effective amount of the compositions can be determined empirically and will depend on the type and severity of the disease, route of administration, disease progression and health, mass and body area of the individual. Such determinations are within the skill of one in the art. The effective amount can also be determined based on in vitro complement activation assays. Examples of dosages of molecules which can be used for methods described herein include, but are not limited to, an effective amount within the dosage range of any of about 0.01 mg/kg to about 300 mg/kg, or within about 0.1 mg/kg to about 40 mg/kg, or with about 1 mg/kg to about 20 mg/kg, or within about 1 mg/kg to about 10 mg/kg. In some embodiments, the amount of composition administered to an individual is about 10 mg to about 500 mg per dose, including for example any of about 10 mg to about 50 mg, about 50 mg to about 100 mg, about 100 mg to about 200 mg, about 200 mg to about 300 mg, about 300 mg to about 500 mg, about 500 mg to about 1 mg, about 1 mg to about 10 mg, about 10 mg to about 50 mg, about 50 mg to about 100 mg, about 100 mg to about 200 mg, about 200 mg to about 300 mg, about 300 mg to about 400 mg, or about 400 mg to about 500 mg per dose.

The compositions may be administered in a single daily dose, or the total daily dose may be administered in divided dosages of two, three, or four times daily. The compositions can also be administered less frequently than daily, for example, six times a week, five times a week, four times a week, three times a week, twice a week, once a week, once every two weeks, once every three weeks, once a month, once every two months, once every three months, or once every six months. The compositions may also be administered in a sustained release formulation, such as in an implant which gradually releases the composition for use over a period of time, and which allows for the composition to be administered less frequently, such as once a month, once every 2-6 months, once every year, or even a single administration. The sustained release devices (such as pellets, nanoparticles, microparticles, nanospheres, microspheres, and the like) may be administered by injection or surgical implantation in various locations.

Dosage amounts and frequency will vary according the particular formulation, the dosage form, and individual patient characteristics. Generally speaking, determining the dosage amount and frequency for a particular formulation, dosage form, and individual patient characteristic can be accomplished using conventional dosing studies, coupled with appropriate diagnostics.

### Unit Dosages, Articles of Manufacture, and Kits

Also provided are unit dosage forms of compositions, each dosage containing from about 0.01 mg to about 50 mg, including for example any of about 0.1 mg to about 50 mg, about 1 mg to about 50 mg, about 5 mg to about 40 mg, about 10 mg to about 20 mg, or about 15 mg of the targeted molecule. In some embodiments, the unit dosage forms of targeted molecule composition comprise about any of 0.01 mg-0.1 mg, 0.1 mg-0.2 mg, 0.2 mg-0.25 mg, 0.25 mg-0.3 mg, 0.3 mg-0.35 mg, 0.35 mg-0.4 mg, 0.4 mg-0.5 mg, 0.5 mg-1.0 mg, 10 mg-20 mg, 20 mg-50 mg, 50 mg-80 mg, 80 mg-100 mg, 100 mg-150 mg, 150 mg-200 mg, 200 mg-250 mg, 250 mg-300 mg, 300 mg-400 mg, or 400 mg-500 mg targeted inhibitor molecule. In some embodiments, the unit dosage form comprises about 0.25 mg targeted molecule. The term “unit dosage form” refers to a physically discrete unit suitable as unitary dosages for an individual, each unit containing a predetermined quantity of active material calculated to produce the desired therapeutic effect, in association with a suitable pharmaceutical carrier, diluent, or excipient. These unit dosage forms can be stored in suitable packaging in single or multiple unit dosages and may also be further sterilized and sealed.

The present invention also provides kits comprising compositions (or unit dosages forms and/or articles of manufacture) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### Uses of Targeted Molecules and Compositions Thereof

The targeted molecules described herein can function to specifically inhibit at least one of p-selectin and complement signaling in the complement pathway and inflammatory manifestations that accompany it, such as recruitment and activation of macrophages, neutrophils, platelets, and mast cells, edema, tissue damage, and direct activation of local and endogenous cells. Therefore, in some embodiments, the invention includes methods of administering a composition comprising at least one targeted molecule described herein to specifically inhibit at least one of p-selectin signaling, complement signaling or an inflammatory manifestation associated with p-selectin signaling or complement signaling.

In some embodiments, the compositions comprising the targeted molecules described herein can be used for diagnosis, treatment or prevention of diseases or conditions that are mediated by excessive or uncontrolled activation of at least one of p-selectin and the complement system, particularly diseases or conditions mediated by excessive or uncontrolled activation of complement signaling. In some embodiments, there are provided methods of treating diseases involving local inflammation process. Exemplary diseases and disorder that can be treated using the compositions and methods of the invention include, but are not limited to ischemia, reperfusion injury, traumatic brain injury, intracranial hemorrhage, including germinal matrix hemorrhage (GMH) and intraventricular hemorrhage (IVH), post-hemorrhagic hydrocephalus (PHH), coronary artery disease, acute myocardial infarction, stroke, and peripheral artery diseases, allergy, asthma, any autoimmune diseases, celiac disease, glomerulonephritis, hepatitis, inflammatory bowel disease, transplant rejection, coagulopathies, thrombotic disorders, CNS injury, diseases of the CNS and peripheral nervous system, neurodegenerative disorders, ocular disorders, including glaucoma and age-related macular degeneration, infectious disease and pathologies of infectious disease (including but not limited to viral and bacterial infections, systemic organ involvement), blood and clotting disorders and inflammatory diseases and disorders.

In some embodiments, there is provided a method of treating a disease in which at least one of p-selectin and complement signaling is implicated in an individual, comprising administering to the individual an effective amount of a composition comprising a targeted molecule comprising: a) a targeting portion comprising an antibody or a fragment thereof, and b) an inhibitor portion comprising an inhibitor (for example a complement inhibitor) or a fragment thereof. In some embodiments, there is provided a method of inhibiting complement activation in an individual having a disease associated with complement activation, comprising administering to the individual an effective amount of a composition comprising a targeted molecule comprising: a) a targeting portion comprising an antibody or a fragment thereof, and b) an inhibitor portion comprising an inhibitor molecule or a fragment thereof. In some embodiments, there is provided a method of inhibiting inflammation in an individual having a disease associated with complement activation, comprising administering to the individual an effective amount of a composition comprising a targeted molecule comprising: a) a targeting portion comprising an antibody or a fragment thereof, and b) an inhibitor portion comprising an inhibitor or a fragment thereof.

In some embodiments, the disease to be treated is ischemia reperfusion injury. Ischemia reperfusion (I/R) injury refers to inflammatory injury to the endothelium and underlying parenchymal tissues following reperfusion of hypoxic tissues. Ischemia reperfusion injury can result in necrosis and irreversible cell injury. The complement pathway (including the alternative complement pathway) is a major mediator of IR injury. Methods provided herein are thus useful for treatment of ischemia reperfusion that occurs in any organ or tissues, such as ischemia-reperfusion injury of any transplanted organ or tissue. Other conditions and diseases in which ischemia-reperfusion injury occurs will be known to those of skill in the art.

In one aspect, the disease or disorder to be treated is germinal matrix hemorrhage (GMH) or post-hemorrhagic hydrocephalus (PHH) that may occur after GMH. GMH refers to a disease of infancy that affects neonates who are born premature or underweight. In certain instances, GMH occurs in a region of the brain near the ventricles called the subventricular zone that contains fragile blood vessels. In certain instances, once the germinal matrix hemorrhage occurs, PHH becomes a significant risk. In the acute period, PHH can occur from physical obstruction of the ventricles by blood products. However, after blood products are cleared or broken down, an inflammatory response remains and creates damage to the walls of the ventricles as well as forces increased production of cerebrospinal fluid by the choroid plexus. Chronically, there is still evidence of cyclic inflammation that results in scar formation, white matter loss, and loss of the ependymal lining within the ventricles. The result is chronic, irreversible hydrocephalus and pathologies that lead to cerebral palsy and severe neurodevelopmental delay. In one embodiment, the method comprises administering one or more compositions described herein to a subject having GMH or PHH. In one embodiment, the subject is an infant. In one embodiment, the subject is premature neonate or a neonate born underweight.

In one aspect, the present invention provides a method of treating a subject having, or who has had, an ischemic stroke, traumatic brain injury, or spinal cord injury. In certain embodiments, the method comprises administering to the subject one or more of the targeted molecules described herein. In certain embodiments, the method comprises the use of one or more targeted molecules described herein as an adjuvant therapy in combination with one or more standard therapies. For example, in certain embodiments, the one or more targeted molecules are used in combination with rehabilitation therapy.

Exemplary types of rehabilitation therapy include, but is not limited to, motor therapy, mobility training, constraint-induced therapy, range-of-motion therapy, electrical and magnetic stimulation, robot-assisted therapy, physical therapy, occupational therapy, speech therapy, cognitive therapy, visual rehabilitation and the like.

In certain aspects, the composition is administered to the subject within 1 minute, 5 minutes, 10 minutes, 15 minutes, 30 minutes, 1 hour, 2 hours, 4 hours, 6 hours, 8 hours, 12 hours, 18 hours, 1 day, 2 days, 3 days, 4 days, 5 days, 6 days, 7 days, 10 days, 2 weeks, 4 weeks, or more following the onset of ischemic injury. In certain aspects the use of the composition as an adjuvant therapy in combination with one or more other therapies increases the therapeutic window for the treatment of ischemic injury.

### Administration

The compositions described herein can be administered to an individual via any route, including, but not limited to, intravenous (e.g., by infusion pumps), intraperitoneal, intraocular, intra-arterial, intrapulmonary, oral, inhalation, intravesicular, intramuscular, intra-tracheal, subcutaneous, intrathecal, transdermal, transpleural, topical, inhalational (e.g., as mists of sprays), mucosal (such as via nasal mucosa), gastrointestinal, intraarticular, intracisternal, intraventricular, rectal (i.e., via suppository), vaginal (i.e., via pessary), intracranial, intraurethral, intrahepatic, and intratumoral. In some embodiments, the compositions are administered systemically (for example by intravenous injection). In some embodiments, the compositions are administered locally (for example by intraarterial or intraocular injection in intracerebral injection).

### Combination Therapy

In some embodiments, provided pharmaceutical formulations are administered to a subject in combination with one or more other therapeutic agents or modalities, for example, useful in the treatment of one or more diseases, disorders, or conditions treated by the relevant provided pharmaceutical formulation, so the subject is simultaneously exposed to both. In some embodiments, a composition is utilized in a pharmaceutical formulation that is separate from and distinct from the pharmaceutical formulation containing the other therapeutic agent. In some embodiments, a composition is admixed with the composition comprising the other therapeutic agent. In other words, in some embodiments, a composition is produced individually, and the composition is simply mixed with another composition comprising another therapeutic agent.

The particular combination of therapies (substances and/or procedures) to employ in a combination regimen will take into account compatibility of the desired substances and/or procedures and the desired therapeutic effect to be achieved. In some embodiments, provided formulations can be administered concurrently with, prior to, or subsequent to, one or more other therapeutic agents (e.g., desired known immunosuppressive therapeutics).

It will be appreciated that the therapies employed may achieve a desired effect for the same disorder or they may achieve different effects. In some embodiments, compositions in accordance with the invention are administered with a second therapeutic agent.

As used herein, the terms “in combination with” and “in conjunction with” mean that the provided formulation can be administered concurrently with, prior to, or subsequent to, one or more other desired therapeutics such as a rehabilitation therapy. In general, each substance will be administered at a dose and/or on a time schedule determined for that agent.

In certain embodiments, the method comprises administering one or more compositions. For example, in one embodiment, the method comprises administering a first composition comprising a fusion protein, antibody, fragment thereof, or nucleotide molecule encoding the same, and a second composition comprising a therapeutic molecule for a disease or disorder associated with inflammation. The different compositions may be administered to the subject in any order and in any suitable interval. For example, in certain embodiments, the one or more compositions are administered simultaneously or near simultaneously. In certain embodiments, the method comprises a staggered administration of the one or more compositions, where a first composition is administered and a second composition administered at some later time point. Any suitable interval of administration which produces the desired therapeutic effect may be used.

### Use in Immunoassays

In some embodiments, the p-selectin binding and p-selectin blocking proteins, peptides, antibodies, antibody fragments and fusion molecules of the invention can be used in assays in vivo or in vitro for detecting the presence of p-selectin or inhibiting p-selectin.

Exemplary assays the p-selectin binding and p-selectin blocking proteins, peptides, antibodies, antibody fragments and fusion molecules can be incorporated into include, but are not limited to, Western blot, dot blot, surface plasmon resonance methods, various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY.

Unless otherwise defined, all technical and scientific terms used herein have the same meaning as commonly understood by one of ordinary skill in the art to which this disclosure pertains. In case of conflict, the present document, including definitions, will control. Exemplary methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., compositions and methods for treating or preventing or detecting or monitoring a complement-associated disorder, will be apparent from the description, the examples, and from the claims.

### EXAMPLES

### Example 1: Novel P-Selectin Targeted Complement Inhibition Reduces Injury Following Hindlimb Ischemia/Reperfusion and Transplantation

Vascularized Composite Allotransplantation (VCA) has become a clinical reality over the past two decades due to the advent of novel immunosuppressive agents¹⁹. Despite these advances the majority of these transplants undergo an episode of acute rejection⁸. This has lead to the investigation of a variety of immunomodulatory strategies to prevent acute rejection and extend allograft survival. However, few studies have addressed mitigating the effects of ischemia-reperfusion injury (IRI), which initiates a cascade of events that leads to a robust alloimune response.

Upon harvesting, the donor VC allograft undergoes a period of cold and warm ischemia. VC allografts may be even more susceptible to IRI as compared to other solid organ transplants (SOT) due to their heterogenous nature and multiple tissue types with varying immunogenicity (Caterson et al., 2013, J Craniofac Surg, 24(1):51-56). While ischemic injury is a result of ATP-depletion and metabolic disturbances that lead to parenchymal cell death, the subsequent injury as a result of organ reperfusion occurs via a broad nonspecific innate immune response (Caterson et al., 2013, J Craniofac Surg, 24(1):51-56; The Science of Reconstructive Transplantation, link.springer.com/book/10.1007%2F978-1-4939-2071-6). Complement is the major effector of the innate immune response and exploiting this pathway as a therapeutic target is crucial in the efforts to reduce IRI. Current therapeutic approaches to reduce IRI in SOT include mainly pulsatile perfusion, preservations solutions, and antioxidants (Caterson et al., 2013, J Craniofac Surg, 24(1):51-56). Of these, few have made substantial clinical impact, though they may provide additional future avenues for treatment as part of a multifaceted approach to reduce IRI in VCA. Others have focused on complement inhibition with some success in multiple preclinical models of SOT (Grafals et al., 2019, Front Immunol, 10). For instance, a small molecule C5a receptor antagonist was successful in reducing IR mediated injury in cardiac and renal allografts in animal models (Vakeva et al., 1998, Circulation, 97(22):2259-2267; van der Pals et al., 2010, BMC Cardiovasc Disord, 10:45; Arumugam et al., 2003, Kidney Int, 63(1):134-142; De Vries et al., 2003, Transplantation, 75(3):375-382). Monoclonal antibodies against the lectin pathway and alternative pathway have also reported some success in pre-clinical cardiac and renal allografts (Schwaeble et al., 2011, Proc Natl Acad Sci USA., 108(18):7523-7528; Thurman et al., 2006, J Am Soc Nephrol JASN, 17(3):707-715). Although these studies have had promising results, systemic complement inhibition may lead to many adverse effects.

While complement inhibition has demonstrated favorable results in reducing IRI in pre-clinical animal models of SOT, complement overall carries immunoprotective effects and systemic inhibition is suboptimal. A novel targeted complement inhibitor in VCA was previously developed using CR2-mediated targeting, which binds to C3 deposition products and allows for targeting of complement inhibition at the site of complement activation (Zhu et al., 2017, Transplantation, 101(4):e75-e85). Additionally, the experiments showed that specific targeting of complement inhibition improves bioavailability and efficacy (Zhu et al., 2017, Transplantation, 101(4):e75-e85). In this study, novel p-selectin (PSel) targeted complement inhibitors (NB.PSelscFv-Crry and B.PSelscFv-Crry) were investigated, which not only inhibits complement, but targets another crucial adhesion molecule involved in the initial inflammatory response to IR, PSel.

Adhesion molecules, such as PSel, play a key role in polymorphonuclear (PMN) cell recruitment to the site of injury and are up-regulated following IRI. Complement activation represents only one of multiple pathways that contribute to the up-regulation of PSel following IR (Atkinson et al., 2006, J Immunol, 177(10):7266-7274). Upon complement activation towards ischemic tissue, C3 is cleaved into C3a and C3b. C3a, C5a, and the cytolytic membrane attack complex (MAC) induce PSel expression, while C3b binds to PSel and induces further complement activation in a positive feedback loop (Atkinson et al., 2006, J Immunol, 177(10):7266-7274). Furthermore, PSel is also expressed on platelets and allows platelet recruitment to the site of injury, which may result in thrombosis (Atkinson et al., 2006, J Immunol, 177(10):7266-7274). Others have shown that blocking PSel functions using an anti-PSel monoclonal antibody effectively reduced microvascular thromboses and improved perfusion in a model of IRI (Klintman et al., 2004, Clin Diagn Lab Immunol, 11(1):56-62). In this study, the effect of PSel blockade on hindlimb VCA perfusion is studied. It is demonstrated that early blockade of PSel with 0.5 mg B.PSelscFv-Crry lead to overall improved hindlimb perfusion by 24 hours in a isograft model (p<0.05) and by day 9 in an allograft model (p<0.05). Though improved perfusion was seen with PSel inhibition, this effect may carry an increased risk of bleeding complications. In addition to improving perfusion, blockade of PSel has also been shown to reduce IRI, which may be related to its effects on complement activation (Atkinson et al., 2006, J Immunol, 177(10):7266-7274). Infusion of an anti-p-selectin monoclonal antibody has been shown to reduce injury related to IR in a rat model of total hepatic ischemia (Garcia-Criado et al., 1995, J Am Coll Surg, 181(4):327-334). In this study, it is shown that by inhibiting the function of PSel and complement activation in a murine model of hindlimb IRI, VCI model, and VCA model, IR-associated injury was reduced, as demonstrated by a reduction in PMN infiltrates, edema, and necrosis within the IR injured tissue.

Due to the success of these experiments, the effect of dual inhibition of complement activation and PSel function on VC allograft survival was evaluated. Following a single administration of 0.5 mg B.PSelscFv-Crry immediately post-transplantation, a significant improvement in allograft survival from 10 to 14 days was observed. This correlates with previous findings that demonstrated that targeted complement inhibition with CR2-Crry improved VC allograft survival from 5.8 days without treatment to 7.4 days with CR2-Crry alone. When combined with subtherapeutic doses of cyclosporine A (CsA) survival following administration of CR2-Crry was further improved to 17.2 days as compared to 7.4 days with CsA alone (Zhu et al., 2017, Transplantation, 101(4):e75-e85). However, in the current study, B.PSelscFv-Crry alone improved graft survival to a similar degree as CR2-Crry in combination with CsA. This suggests that blockade of PSel in addition to complement may have a greater impact on the severity of IRI.

In conclusion, the novel fusion proteins presented here allowed for IRI site-specific drug delivery of a dual functioning complement and PSel inhibitor. Of the proteins tested, B.PSelscFv-Crry represents the most promising therapeutic candidate for the further development of immunosparing regimens. However, further investigation is required to determine whether an even greater improvement in allograft survival may be achieved in combination with subtherapeutic doses of conventional immunosuppression. Additionally, due to the potential for bleeding complications with inhibition of PSel, further comparative studies are needed with the PSel non-blocking construct. While the NB.PSelscFv-Crry could potentially reduce the risk of bleeding complications, it was not as effective in reducing IRI, but may prove to be useful in combination with subtherapeutic doses of conventional immunosuppression. Ultimately, a multifaceted approach in innate immune suppression may offer the best potential to reduce injury associated with IR, allow for immunosparing regimens, and improve allograft survival. While future studies are needed to exploit the full potential of these novel targeted complement inhibitors, they may pave the way for VCA to become a more clinically viable option.

The materials and methods used in the experiments are now described.

Construction of Expression Plasmids, Protein Expression, and Protein Purification

RNA Isolation

Total RNA was isolated from two different hybridoma cell lines: anti-p-selectin 2.12 (blocking) and anti-p-selectin 2.3 (non-blocking) by using TRIzol Reagent (Invitrogen™) according to the manufacturer's instructions. mRNA was isolated by using the Oligotex mRNA mini kit from Qiagen (Cat No.: 70022).

RT-PCR and Gene Cloning

RT-PCR was performed to acquire cDNA from hybridoma mRNA. Briefly, mRNA samples were heated at 70° C. for 5 minutes and quickly cooled on ice before mixed with RT reaction mixture. RT-PCR was carried out in a 20 l volume containing mRNA, random primer, dNTPs and reverse transcriptase at 42° C. for 90 minutes. cDNA was used to amplify hybridoma VL and VH fragments with a primer mix from Fisher scientific (Catalog No. 69-831-3 MiliporeSigma™ Novagen™ Mouse Ig-Primer Set). VL and VH gene were then cloned into pCR™ 2.1 Vector with TA Cloning™ Kit (Invitrogen™, Catalog number: K204040). The VL and VH were sequenced with Genewiz and the confirmed sequences were synthesized by the Genewiz company with the linker of (Gly4Seri)₃ between the VH and VL fragments. Next, the scFv gene was attached to the Crry gene with the linker of (Gly4Ser₁)₂ and the scFv-Crry fusion gene was cloned into the pEE12.4 vector (Lonza).

Protein Expression and Purification

Expi293 cells (A14527, ThermoFisher) were used to express the proteins. Plasmids were transfected into Expi293 cells. The viability of the cells was analyzed in a cell counter or microscope the day after transfection. To assess protein expression, aliquots of the cell media containing transfected cells were collected at regular intervals to determine the optimal harvesting day. The collected aliquots were spun at 13000 g for 5 min, and the supernatant was analyzed with a dot-blot. Seven days after transfection, the cells were harvested by centrifugation at 2000 g for 15 minutes. The supernatant was then filtered through a 0.22 m filter and loaded on the His60 Nickle column (Clontech, 635664) for purification. The fusion proteins were concentrated to a concentration of 1.0-30.0 mg/ml with Amicon ultra centrifugal filters (Merck Millipore) with a 30 kDa molecular weight cut-off. At the same time, the buffer was exchanged into antibodies working buffer PBS. Fusion proteins were aliquoted into a small tube after functional testing for p-selectin binding and complement inhibition.

In Vitro Characterization of Recombinant Proteins

Binding of fusion proteins to plate bound p-selectin was determined by enzyme-linked immunosorbent assay (ELISA) (Abcam). Non-blocking and blocking fusion proteins (NB.PselscFv-Crry and B.PselscFv-Crry, respectively) were plated at varying doses in addition to varying doses of C3d-Crry as a negative control. Complement inhibition was measured for NB.PselscFv-Crry and B.PselscFv-Crry using flow cytometic analysis of C3 deposition on zymosan A particles (SigmaAldrich) as previously described (Atkinson et al., 2005, Clin Invest, 115(9):2444-2453). Another complement inhibitor that was previously described, CR2-Crry, was used as a positive control (Atkinson et al., 2006, J Immunol, 177(10):7266-7274). The extent of complement inhibition was normalized by the subtraction of post-IRI complement desposition levels from their baseline levels.

Hindlimb IRI

Adult male C57BL/6 mice (The Jackson Laboratory) aged 8-10 weeks and weighing 20-25 g were anesthetized with 7.5 mg/kg ketamine and 10 mg/kg xylazine by i.p. injection. Animal respirations were continuously monitored throughout the experiment and their body heat was maintained with a heating pad. A rubber band was placed around the right hindlimb for 2 hours of ischemia time, then subsequently removed. Immediately upon reperfusion, mice were either injected with 0.1 ml vehicle control (PBS), 0.25 mg NB.PselscFv-Crry, or 0.25 mg B.PselscFv-Crry treatments by i.p. injection. Mice were allowed to recover from anesthesia under a heating lamp. At 24 hours post-reperfusion mice were sacrificed by cervical dislocation under ketamine anesthesia. Blood and hindlimb tissue was collected for analysis. Animal procedures were approved by the Medical University of South Carolina Animal Care and Use Committee (IACUC).

Fluorescent Labeling Biodistribution

The hindlimb IRI model (described in detail above) was used to evaluate biodistribution of both fusion proteins. NB.PselscFv-Crry and B.PselscFv-Crry were fluorescently labeled using the *** kit. Following 2 hours of ischemia time, 0.1 ml vehicle control, 0.25 mg fluorescently labeled NB.PselscFv-Crry, or 0.25 mg fluorescently labeled B.PselscFv-Crry were injected by IP injection into each mouse. At 24 hours post-reperfusion mice were imaged using CRi's near-infrared Meastro in-vivo fluorescent imaging system. Animal procedures were approved by the Medical University of South Carolina Animal Care and Use Committee (IACUC).

Histopathology

Tissue specimens for staining were taken from de-boned hindlimb specimens and either frozen in liquid nitrogen and placed in −80° C. or fixed in 10% formalin at 4° C. overnight and subsequently processed to paraffin. Sections from each of the hindlimbs were stained with H&E and scored for muscle necrosis, neutrophil infiltrate, and edema. A score of 0 was assigned to normal muscle,***. All histological examinations were carried out in a blinded fashion.

Animals

Male C57BL/6 (B6, B6J) mice at 10-12 weeks of age were used as both donor and recipient representing an MHC identical match, were purchased from the Jackson Laboratory (JAX). All animals were housed in the animal facility of the Medical University of South Carolina, under pyrogen-free conditions, with temperature and lighting cycles controlled, and water and commercial mice chow freely available. When applicable, the animals were anesthetized with ketamine and xylazine, and euthanized with cervical dislocation under anesthesia. All experiments were conducted in accordance with the Guide for the Care and Use of Laboratory Animals of the National Institutes of Health and following the Institutional Animal Care and Use Committee (IACUC) protocols authorized by the Medical University of South Carolina, Charleston, S.C., with the authorized protocol number of AR00136.

Orthotopic Hindlimb Vascularized Composite Isograft (VCI) Model

A previously described orthotopic hindlimb osteomyocutaneous VCA transplant mouse model was used, with the exception of a syngeneic transplant being performed in this study. Briefly, VCI harvest in the ketamine/xylazine-anesthetized donor mouse began with a circumferential hindlimb incision along the inguinal ligament and the inguinal fat pad was retracted inferiorly. The femoral artery and vein were carefully dissected from the inguinal ligament superiorly to the bifurcation of the saphenous and popliteal artery while ligating small branches, including the lateral circumflex femoral artery. A vascular clamp was applied across the femoral vessels at the distal margin of dissection for the donor and the proximal margin of dissection for the recipient. The femoral vessels were ligated and transected at the end opposite the vascular clamp. Bipolar electrocautery (Bovie Derm 102, Bovie Medical Corporation) was used to transect the thigh musculature. The femur was transected sharply using scissors and the bone marrow cavity was packed with Surgical Fibrillar (Johnson and Johnson Medical Device Companies) to achieve adequate hemostasis. A 27 g and 25 g polyamine cuff was then secured to the femoral artery and vein, respectively, as previously described (Sucher et al., 2010, Transplantation, 90(12):1374-1380). The graft was then flushed with 3 ml heparinized saline, wrapped in saline gauze and placed on ice. The donor mouse was euthanized by cervical dislocation under anesthesia. In recipient mice, the femoral vessels were isolated in a similar fashion to the donor mouse and the muscle and bone were again transected with the bone marrow packed with Surgical Fibrillar for hemostasis. The VCI was then inset using a 21 g needle cut to lcm length as an intramedullary rod and the muscle was re-approximated with 6-0 polysorb suture. The femoral vessels were re-approximated using the previously described microvascular cuff technique and 10-0 nylon sutures for the microvascular anastomosis. The sciatic nerve was re-approximated by one simple interrupted suture using 10-0 nylon. The circumferential inguinal incision was then closed and the animal was allowed to recover. VCIs were then evaluated daily with macroscopic inspection of the limb, Laser Speckle Perfusion Doppler (moor-FLPI2, Moor Instruments), and conventional Laser Doppler Monitor (moorVMS-LDF, Moor Instruments). Histological changes, neutrophilic, and lymphocytic infiltration were evaluated by microscopy after hematoxylin and eosin (H&E) staining and myeloperoxidase (MPO) staining.

Statistical Analysis

All data are presented as mean±SD. All data were subjected to statistical analysis using Prism software version 8 (GraphPad Software Company). Statistical analyses of the data were interpreted by unpaired t test for comparison of two groups. A p value of less than 0.05 was considered significant.

The experimental results are now described.

PSelscFv-Crry Constructs Bind p-Selectin and Inhibit Complement Activation

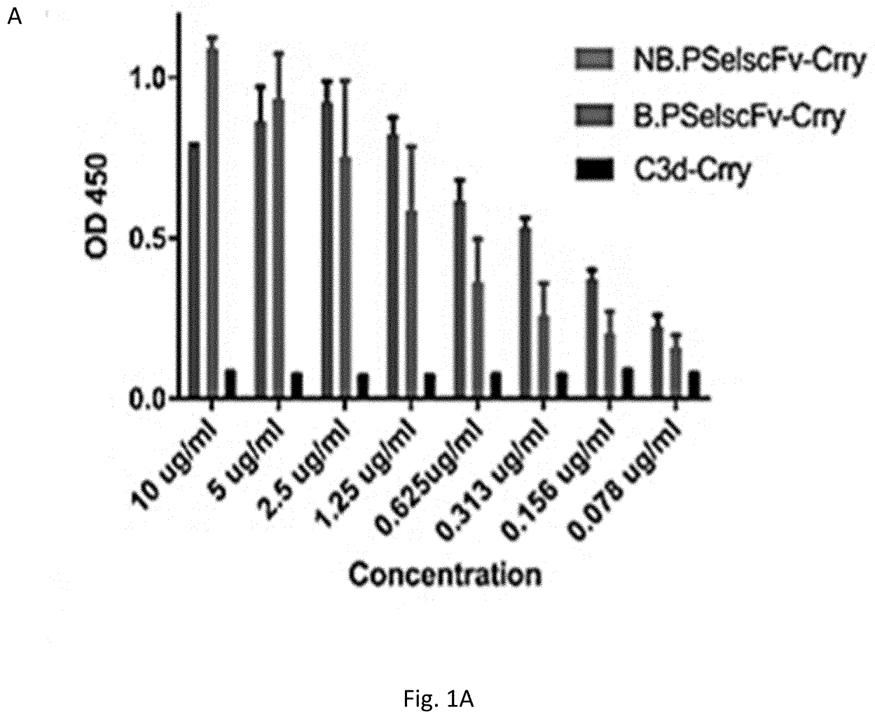

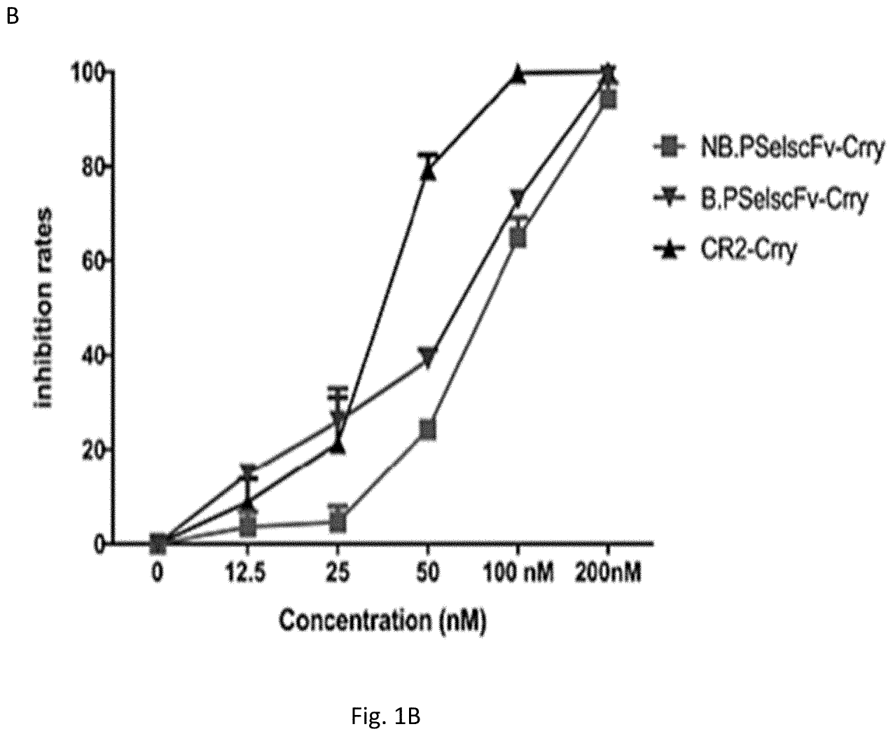

Both NB.PSelscFv-Crry and B.PSelscFv-Crry were characterized in vitro by determining their ability to bind PSel. Both the PSel targeted NB.PSelscFv-Crry and B.PSelscFv-Crry, but not the C3d targeted C3d-Crry, bound to PSel in a dose-dependent fashion (FIG. 1A).

Next, the capability of both NB.PSelscFv-Crry and B.PSelscFv-Crry to inhibit complement activation was evaluated. As previously described (Atkinson et al., 2005, Clin Invest, 115(9):2444-2453), a zymosan assay was performed to measure the activation of the alternative complement pathway by mixing either NB.PSelscFv-Crry or B.PSelscFv-Crry with mouse serum. Inhibition of complement activation by both constructs was observed in a dose-dependent relationship and had similar efficacy to a previously well-characterized complement inhibitor, CR2-Crry (Atkinson et al., 2005, Clin Invest, 115(9):2444-2453). Complement inhibition of 50% or greater was seen at doses of 100 nM or greater for NB.PSel-scFv-Crry and B.PSelscFv-Crry, while only 50 nM CR2-Crry were required for a similar effect (FIG. 1i).

Blocking and Non-Blocking PSelscFv-Crry Reduce IRI In Vivo

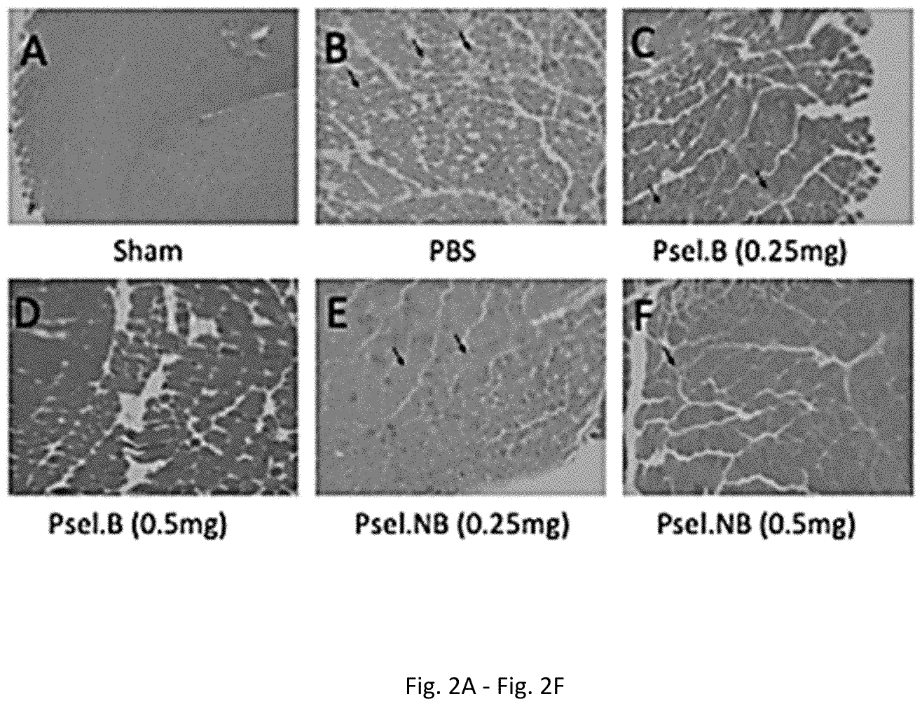

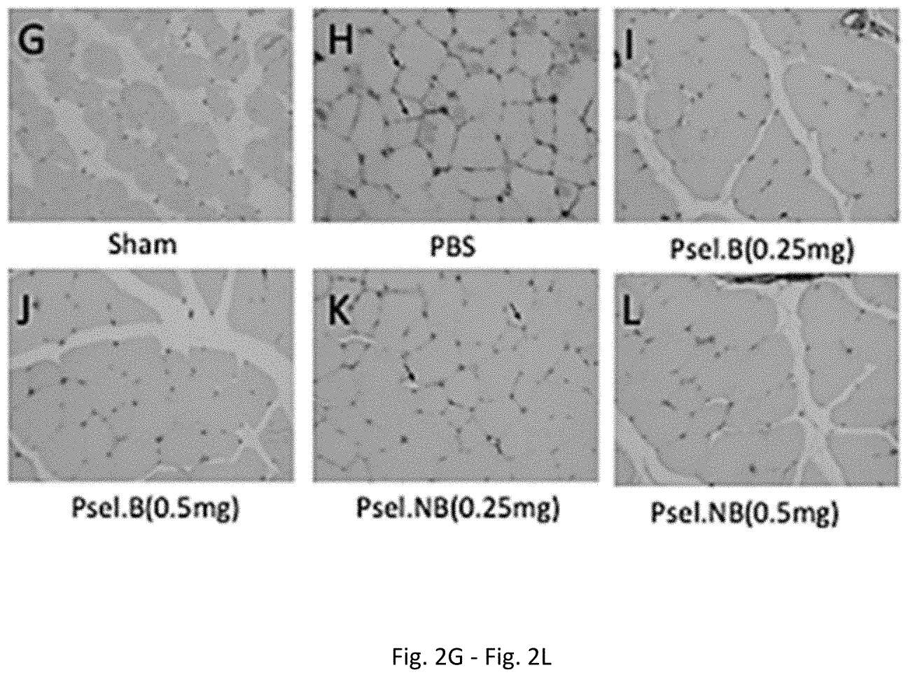

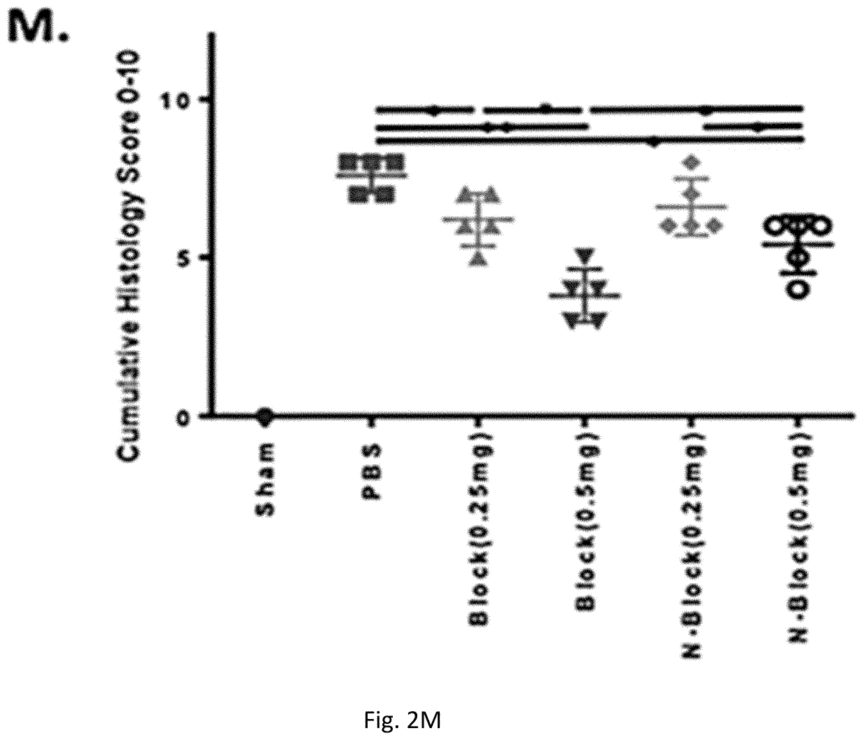

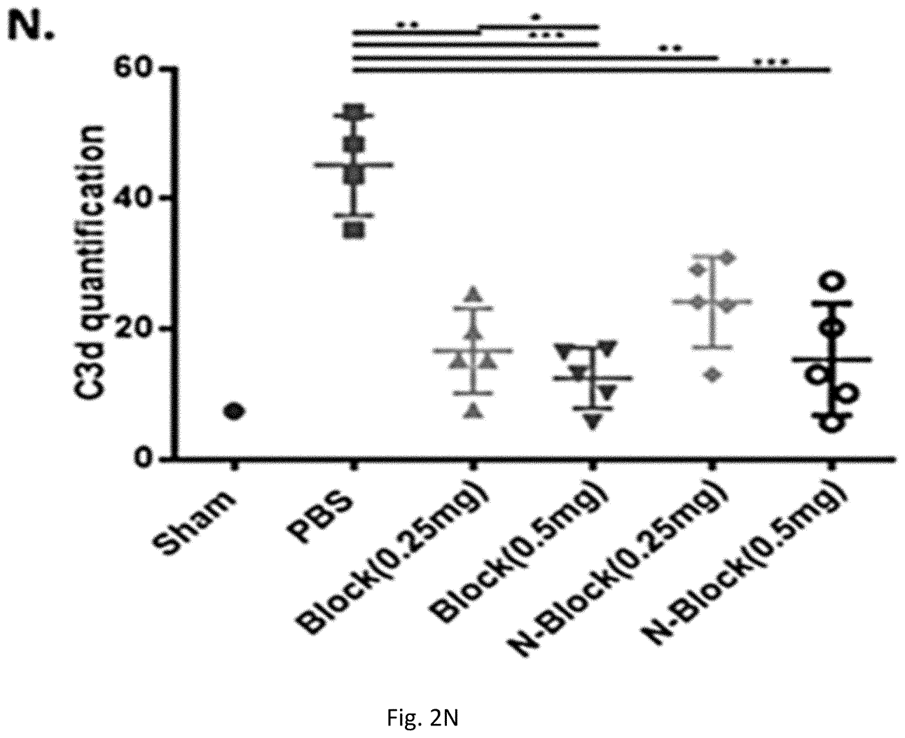

Following ischemia-reperfusion, complement activation is a key initiator of further immune activation and neutrophil trafficking resulting in tissue injury (Ioannou et al., 2011, Clin Immunol, 141(1):3-14). Furthermore, PSel is up-regulated by complement activation products (Atkinson et al., 2006, J Immunol, 177(10):7266-7274). The effect of novel PSel targeted complement inhibitors on tissue injury following ischemia-reperfusion was evaluated using an in vivo hindlimb IRI model. A single dose of 0.25 mg NB.PSelscFv-Crry or B.PSelscFv-Crry was administered via tail vein injection following hindlimb reperfusion. On histologic evaluation, sham-injected mice showed no edema or polymorphonuclear cells (FIG. 2A) in contrast to the vehicle control group (FIG. 2B). Both NB.PSelscFv-Crry and B.PSelscFv-Crry treatment groups (FIG. 2C, FIG. 2D, FIG. 2E, FIG. 2F) showed a reduction in edema and polymorphonuclear infiltrates as compared to the vehicle control group. Furthermore, higher doses of both NB.PSelscFv-Crry and B.PSelscFv-Crry were associated with greater reductions in edema and polymorphonuclear infiltrates (FIG. 2D, FIG. 2F). 0.5 mg B.PSelscFv-Crry was most effective in reducing IRI (FIG. 2D).

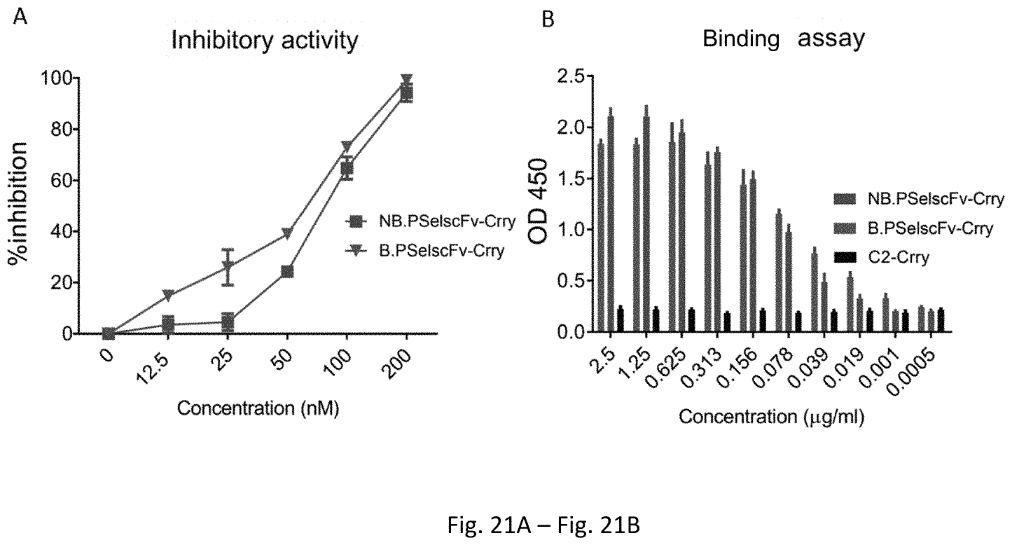

Using immunohistochemistry staining for C3d, complement deposition for each hindlimb section was evaluated. Sham-injected mice showed no C3d deposition (FIG. 2G) in contrast to vehicle control hindlimbs (FIG. 2H), which displayed high amounts of C3d deposition secondary to complement activation. A reduction in C3d deposition was seen following treatment with either 0.25 mg or 0.5 mg of NB.PSelscFv-Crry or B.PSelscFv-Crry (FIG. 21, FIG. 2J, FIG. 2K, FIG. 2L) with 0.5 mg B.PSelscFv-Crry showing the greatest reduction in C3d deposition (FIG. 2J).

In addition, hindlimb injury was quantified by two trained pathologists using a histologic grading scale from 0-5 with scores added for a total of 10 possible points (FIG. 2M, FIG. 2N). No injury, as defined by amount of neutrophilic infiltrates, level of complement C3d deposition, and extent of edema, was seen in the sham-injected mice (average cumulative score of 0 out of 10), whereas high amounts of injury were found in the vehicle control mice (average cumulative score of 7.6 out of 10). Significant reduction in injury was seen in the 0.25 mg NB.PSelscFv-Crry and B.PSelscFv-Crry treatment groups as compared to the vehicle control (p<0.05). Even greater reduction in injury was seen in the 0.5 mg NB.PSelscFv-Crry and B.PSelscFv-Crry treatment groups as compared to the vehicle control (p<0.05). A significant reduction in injury was also observed between the 0.25 mg and 0.5 mg treatment dosages (p<0.05) indicating a dose-dependent relationship for the attenuation of IRI in vivo.

Blocking and Non-Blocking PSelscFv-Crry Exhibit Site-Specific Action In Vivo

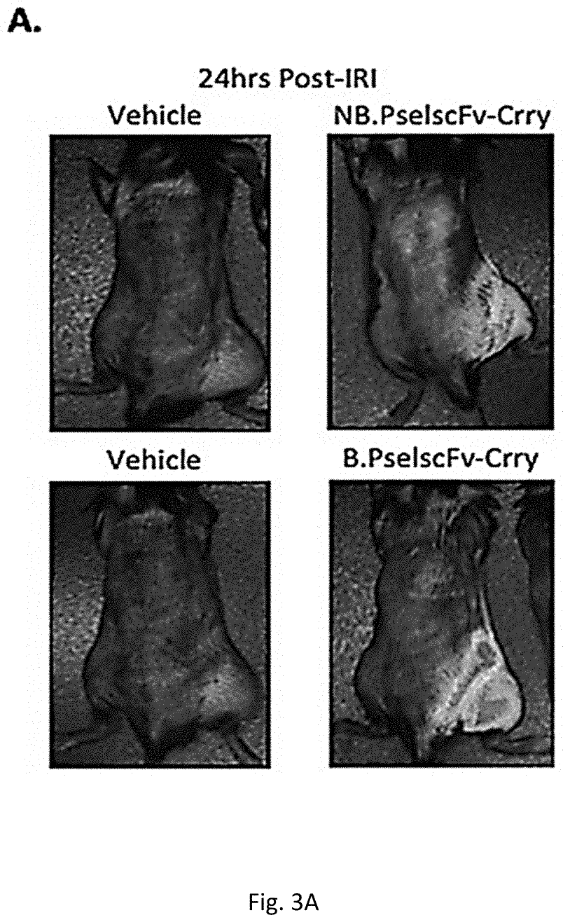

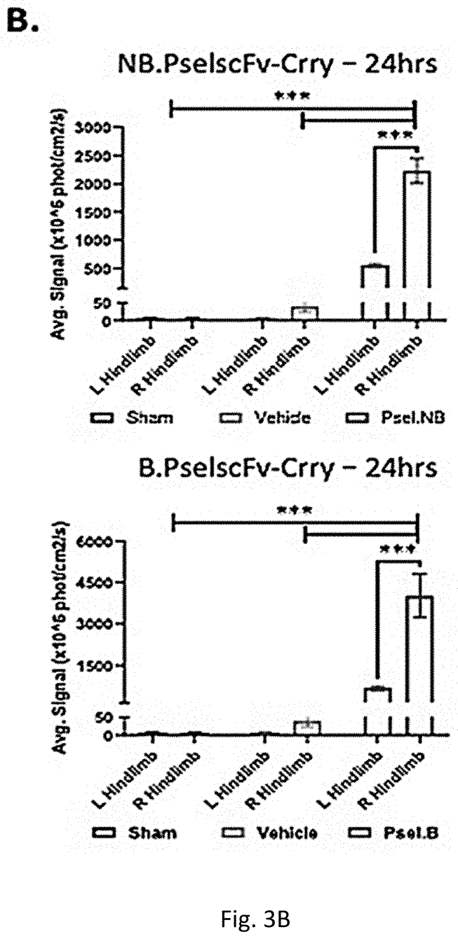

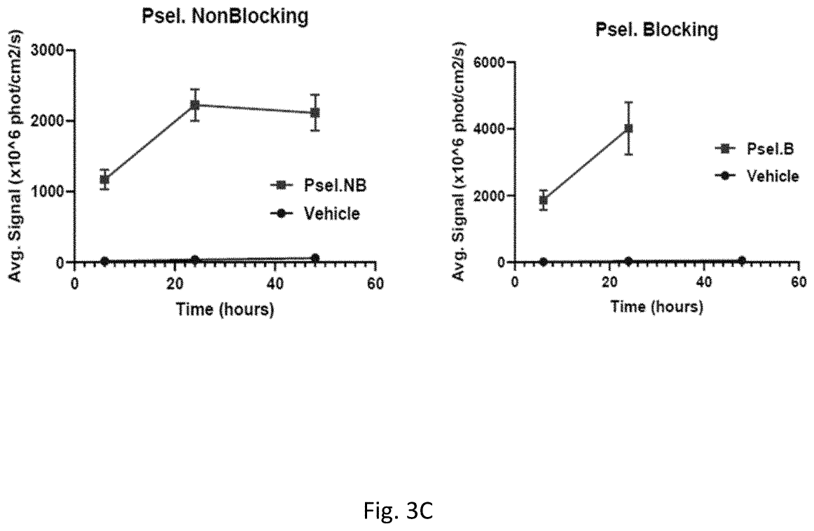

PSel is known to be up-regulated on the surface of endothelial cells following IR (Zhu et al., 2017, Transplantation, 101(4):e75-e85). Therefore, to confirm the targeting specificity of NB.PSelscFv-Crry and B.PSelscFv-Crry following IR, each construct was fluorescently labeled and administered intravenously via tail vein injection to mice upon hindlimb reperfusion following 2 hours of ischemic time. At 24 hours post-reperfusion, Maestro imaging was used to evaluate the biodistribution of each construct in vivo. As compared to vehicle control (PBS), both NB.PSelscFv-Crry and B.PSelscFv-Crry preferentially accumulated at the site of IRI (FIG. 3A). The average fluorescent signal was quantified for each hindlimb in the sham, vehicle control, and treatment groups (FIG. 3B). Average fluorescence was significantly greater in the right hindlimb following IR as compared to the left hindlimb without IR (p<0.001). Additionally, a higher average fluorescence was seen when comparing right hindlimb following IR of the mice treated with NB.PSelscFv-Crry and B.PSelscFv-Crry to hindlimbs of mice in the sham and vehicle control groups (p<0.001), validating protein localization to the injured hindlimb.

Blocking PSelscFv-Crry Improves Hindlimb Perfusion in Vascularized Composite Isograft and Allograft Models

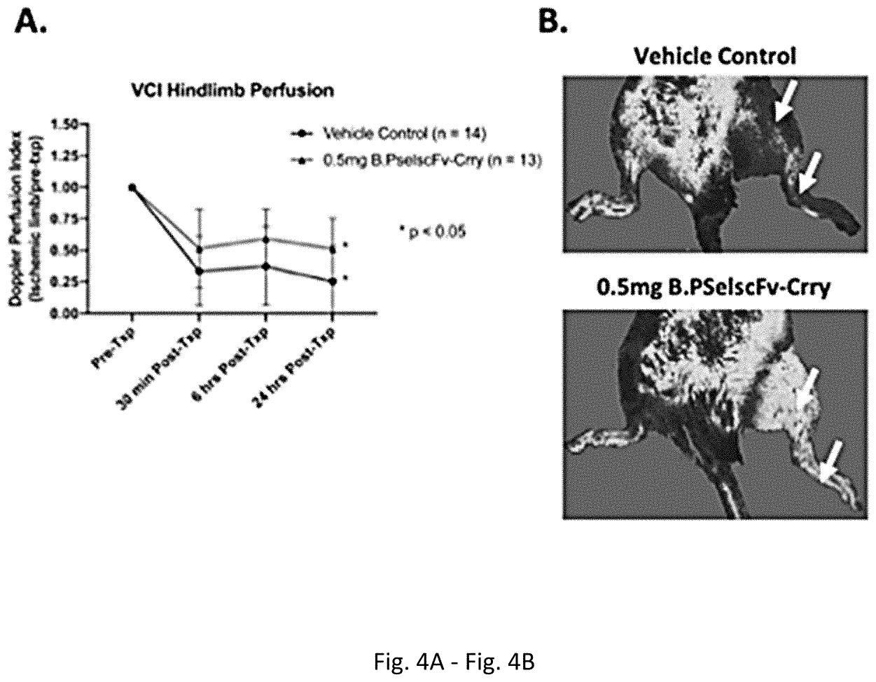

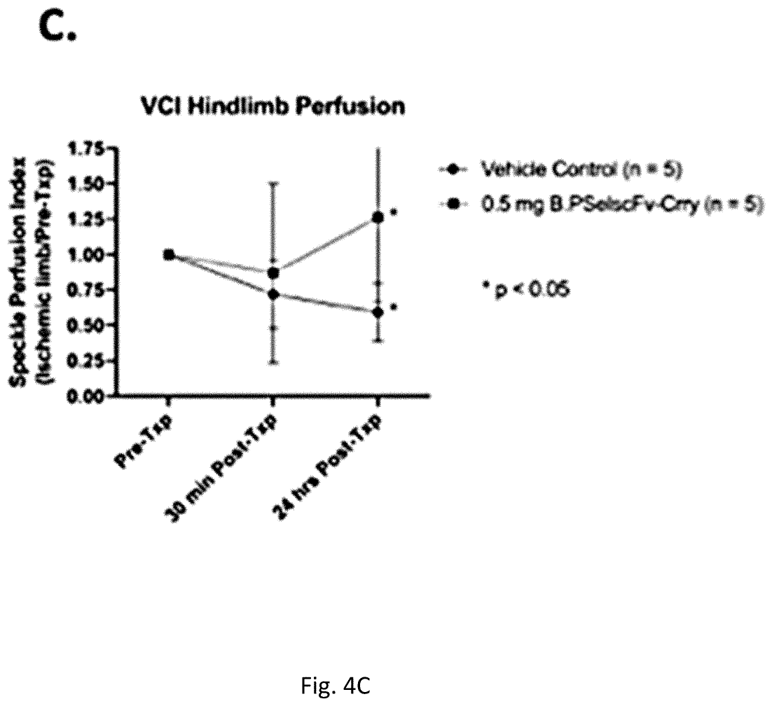

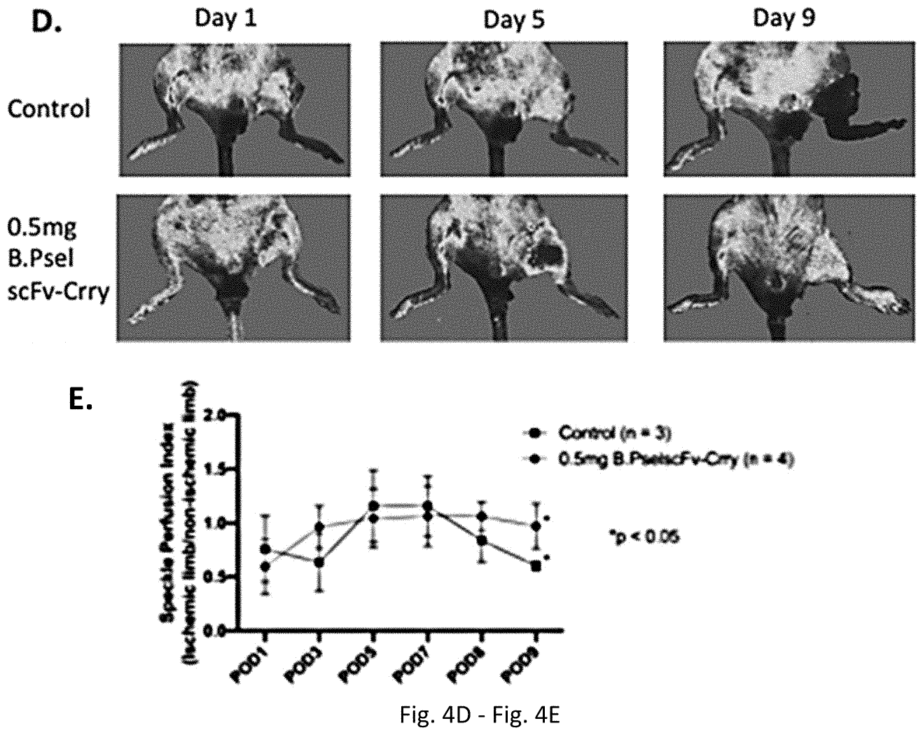

P-selectin antagonism by anti-p-selectin antibodies has been shown to improve microvascular perfusion and attenuate neutrophil accumulation in models of IRI (Klintman et al., 2004, Clin Diagn Lab Immunol, 11(1):56-62; Nagashima et al., 1998, Circulation, 98(19 Suppl):II391-397). Therefore, the effect of B.PSelscFv-Crry was assessed on hindlimb perfusion following transplantation in both isograft and allograft models. Mice in the isograft model treated with a single dose of 0.5 mg B.PSelscFv-Crry immediately post-transplant demonstrated a significant improvement in hindlimb perfusion by 24 hours post-transplant as measured by conventional laser doppler (FIG. 4A) and laser doppler speckle imaging (FIG. 4B, FIG. 4C) (p<0.05). In contrast, B.PSelscFv-Crry treated mice allocated to the allograft model did not show a significant improvement in perfusion at 24 hours post-transplant. However, overall perfusion by postoperative day 9 was significantly improved in the B.PSelscFv-Crry treatment group as compared to the vehicle control shown by laser speckle doppler imaging (FIG. 4D, FIG. 4E) (p<0.05).

Vascularized Composite Isograft IRI is Reduced with B.PSelscFv-Crry Administration

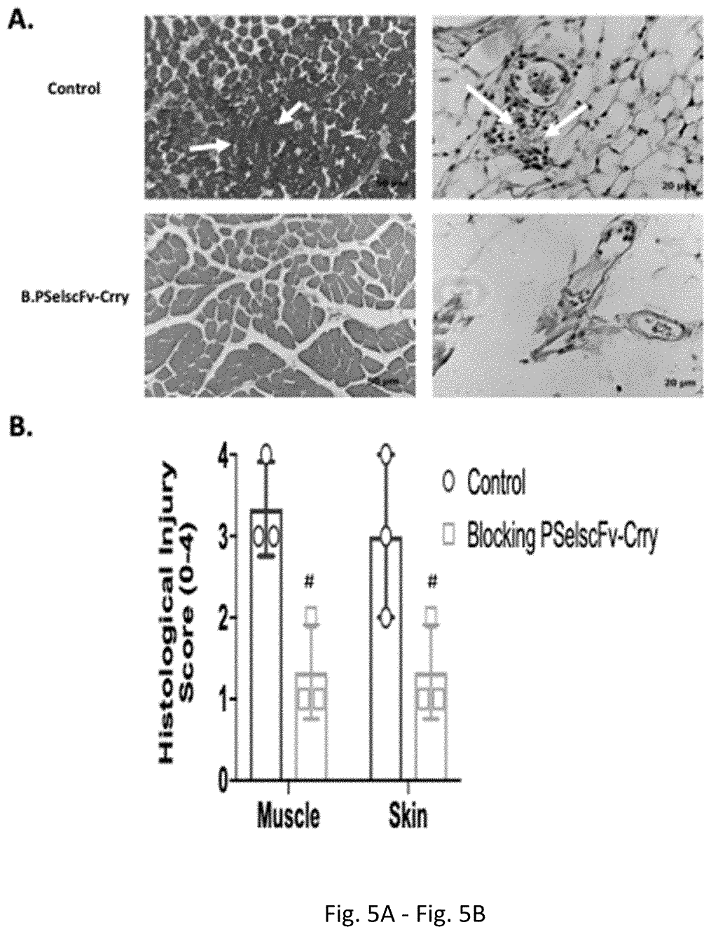

Next, the degree of ensuing injury following reperfusion was evaluated after administration of B.PSelscFv-Crry using a hindlimb VCI model to eliminate any confounding alloimmune response. Increased neutrophilic infiltration and muscle necrosis was observed in the vehicle control group as compared to the B.PSelscFv-Crry treated group at 24 hours post-transplantation (FIG. 5A). The injury was quantified by a trained pathologist using a histopathologic grading system on a scale of 0-4 for both muscle and skin specimens and showed a significant reduction in IRI following B.PSelscFv-Crry treatment (FIG. 5B) (p<0.05).

Blocking PSelscFv-Crry Prolongs Graft Survival of Hindlimb Vascularized Composite Allografts

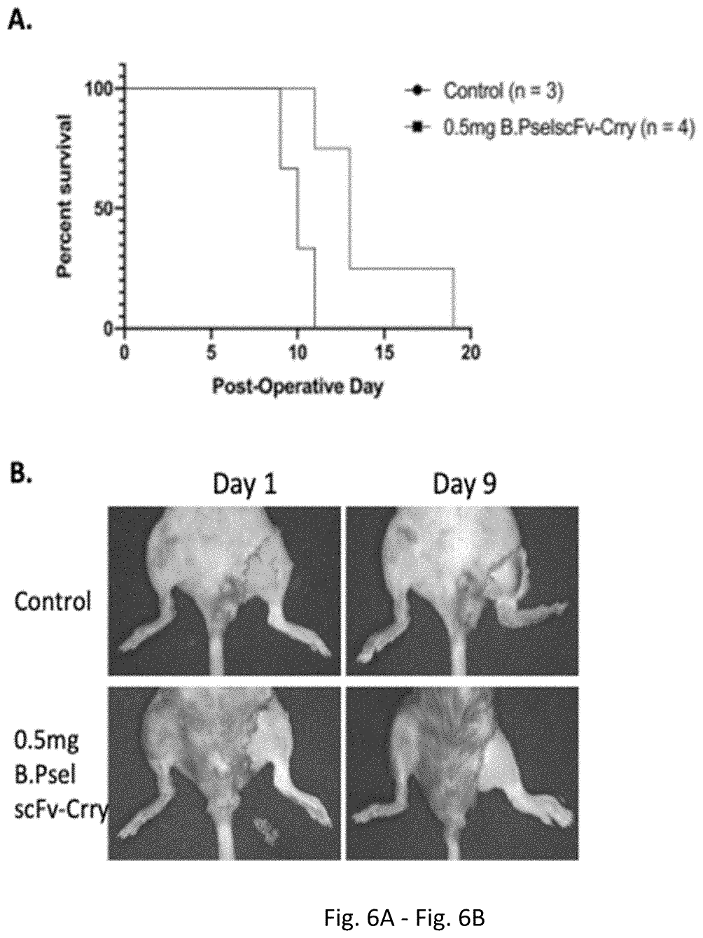

Inhibition of complement-mediated IRI has been shown to reduce the alloimmune response and prolong graft survival in VCA when combined with subtherapeutic doses of conventional immunosuppression (Zhu et al., 2017, Transplantation, 101(4):e75-e85). To evaluate the effect of the novel bi-functional B.PSelscFv-Crry fusion protein on the alloimmune response, allograft survival was examined in a murine orthotopic hindlimb VCA model following early treatment with B.PSelscFv-Crry. The aim was to investigate the sole effect of B.PSelscFv-Crry on allograft survival, and therefore no conventional immunosuppression was administered. Given a single immediate postoperative dose of 0.5 mg B.PSelscFv-Crry a prolonged allograft survival to a mean of 14 days was observed, as compared to a mean allograft survival of 10 days in the vehicle control group (FIG. 6B) (p<0.05). Gross images of representative control and B.PSelscFv-Crry treated groups reveal Banff clinical grade 4 rejection in one control mouse and Banff clinical grade 1 rejection in one treated mouse by day 9 (FIG. 6A).

### Example 2

The experiments presented herein describe an approach to target complement inhibition to sites of inflammation. More specifically, to target a complement inhibitor sites of P-selectin expression. The targeting moiety consists of anti-P-selectin Ab fragments (for eg. scFv, Fab, whole Ab or other derivatives) linked to a complement inhibitor, although other types of therapeutic could similarly be targeted to sites of P-selectin expression. The targeting vehicles described are scFv's derived from mAbs that recognize both mouse and human P-selectin. The P-selectin Abs or derived scFv's may have blocking or non-blocking P-selectin activity (i.e. inhibit or not P-selectin mediated cell binding). The types of construct described have wide potential application for treating injury in general, ischemia reperfusion injury, inflammation, autoimmunity, alloimmunity, coagulopathies, thrombotic disorders, brain and CNS injuries, neurodegenerative conditions, cancer and any disease/condition in which the adhesion molecule P-selectin is expressed or in which blocking the otherwise normal physiological function of P-selectin provides a therapeutic effect. As proof of principle, in vitro data, and data generated in models of hindlimb ischemia reperfusion injury, stroke and traumatic brain injury are provided. The complement inhibitor utilized in these studies described below is murine Crry, but any complement inhibitor of any species could be linked (eg. Human CR1, fH, C4BP, CD59, mAP44, or derivatives thereof).

By adding the therapeutic effect of P-selectin binding and blocking, and because of the novel targeted strategy used, the described inhibitors provide an additional step to the currently investigated technologies and may have higher efficacy in treatment of inflammatory diseases. An additional advantage is that the constructs may prevent P-selectin dependent infiltration of neutrophils to injured tissue, which is a major cause of edema and oxidative stress. For example, in conditions involving the brain, neutrophil infiltration and activation is a key driver of prolonged blood brain barrier dysfunction leading to edema and hemorrhage, which can be fatal. Therefore, targeted inhibition of neutrophil infiltration in addition to complement inhibition may address a new challenge in disease treatment and expand the therapeutically targeted pathophysiological mechanisms.

The 2 scFv's characterized here are 2.12 scFv (Psel2.12, also referred to herein as Psel.B or B.PSelscFv) and P-selectin 2.3 scFv (Psel2.3, also referred to herein as Psel.NB or NB.PSelscFv). The Psel2.12 construct binds to P-selectin and blocks binding of PSGL-expressing cells (eg neutrophils) whereas the Psel2.3 scFv binds to P-selectin without blocking PSGL binding. These mAbs and derived constructs bind mouse and human P-selectin. These different types of scFv are important from both a therapeutic and an investigational standpoint, and allow investigation of whether targeting the complement inhibitor to site of injury is enough/advantageous to provide therapeutic benefit, or additionally blocking the P-selectin binding is an added therapeutic value.

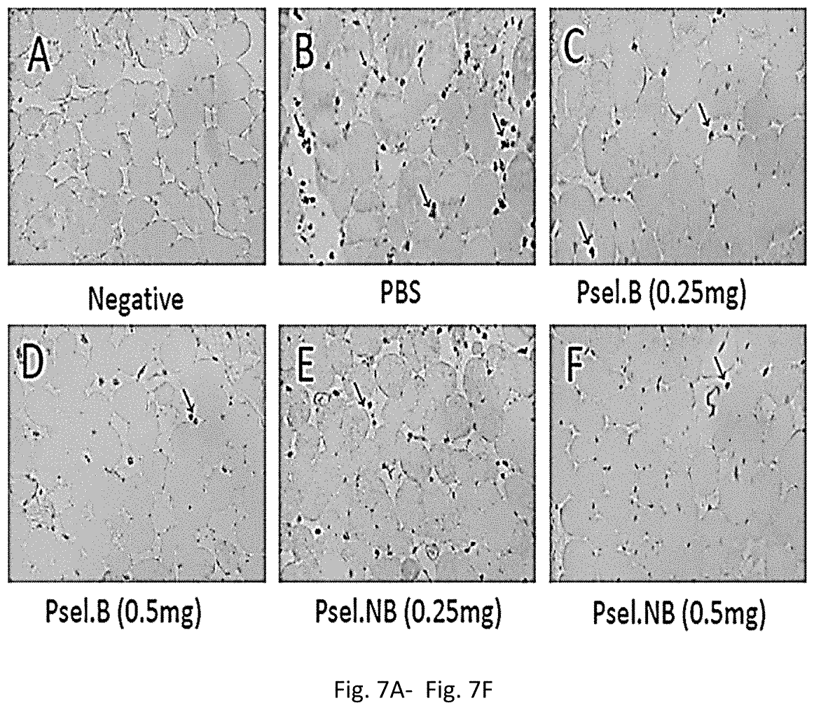

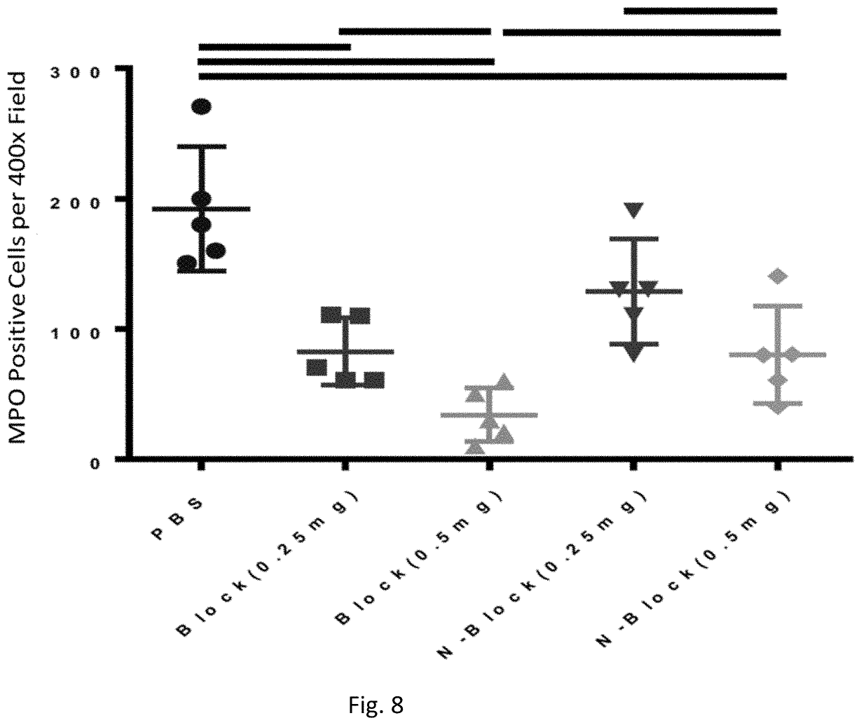

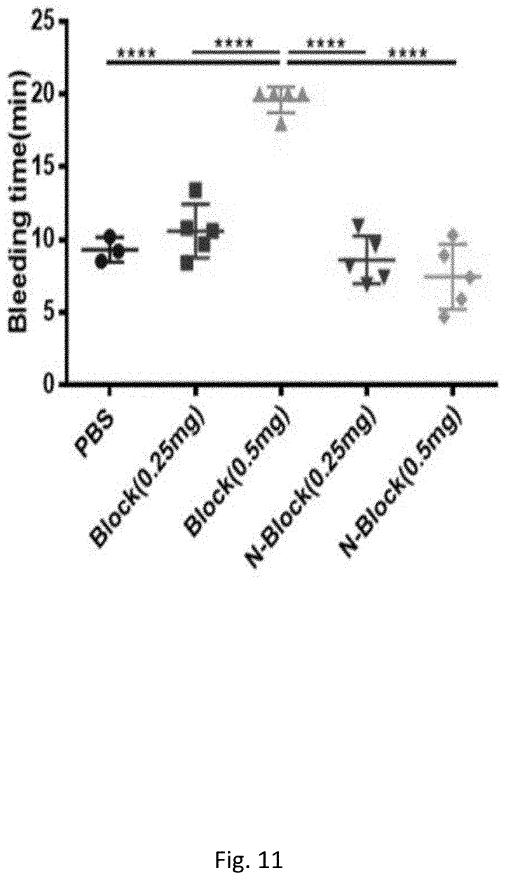

FIGS. 7 and 8 show detection of myeloperoxidase (MPO) in murine hind limb muscle tissue sections after 2 hours of ischemia and 24 hours of reperfusion. No immunostaining was detected in control PBS-treated mice with isotype control Ab (FIG. 7A). Immunostaining of specimens from mice treated with different doses of Psel.B and Psel-NB mice (FIG. 7C, FIG. 7D, FIG. 7E, and FIG. 7F) showed significantly less MPO-positive cells in muscle tissue than from PBS group (FIG. 7B). The black arrows indicate examples of MPO-positive cells. Original magnification×400. FIG. 11 shows a semiquantitative analysis of MPO+ cells. MPO positive cells per 400× field after hindlimb IRI and treatment with different doses of Psel-B and Psel-NB. Pairwise comparisons between hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05), hindlimb IRI+Psel.B (0.25 mg) vs. hindlimb IRI+Psel.B (0.5 mg) (p<0.05), hindlimb IRI+Psel.B (0.5 mg) vs. hindlimb IRI+Psel.NB (0.5 mg) (p<0.05) and hindlimb IRI+Psel.NB (0.25 mg) vs. hindlimb IRI+Psel.NB (0.5 mg) (p<0.05). Differences between hindlimb IRI+Psel.B (0.25 mg) vs. hindlimb IRI+Psel.NB (0.25 mg) and hindlimb IRI+PBS vs. hindlimb IRI+Psel.NB (0.25 mg) were not significant. In vivo analysis shows that the blocking construct reduces infiltration of neutrophils significantly better than non-blocking construct (MPO staining of cells in hindlimb IRI experiment, FIG. 7 and FIG. 8).

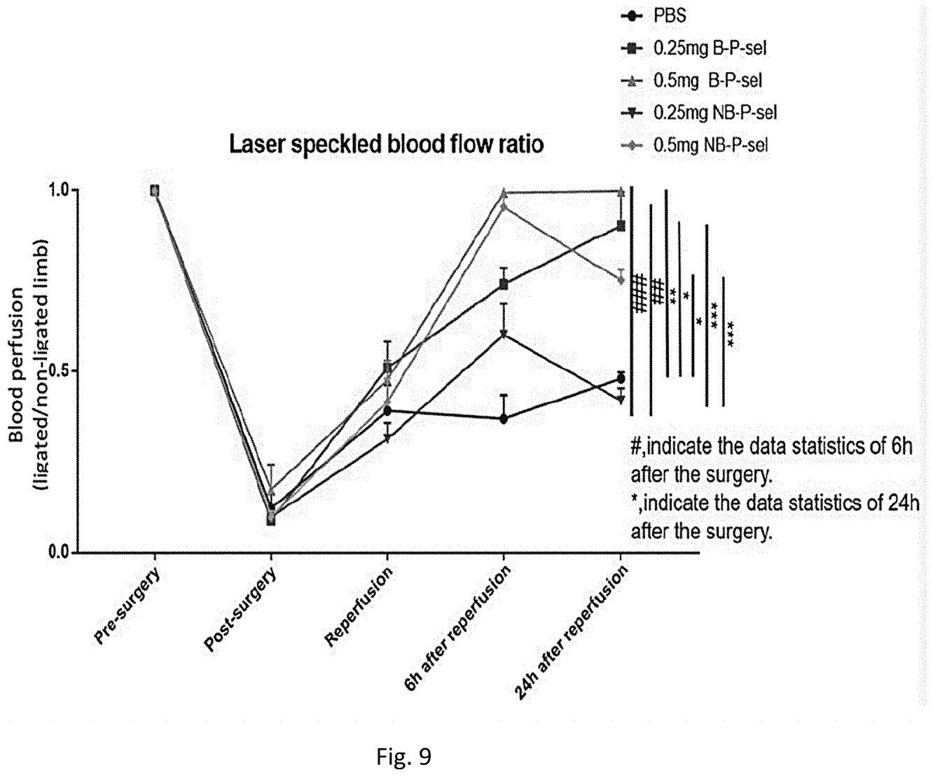

FIG. 9 shows a time course of the recovery of blood flow as the ratio of the ligated to non-ligated hindlimb in hindlimb IRI after treatment with different doses of Psel-B and Psel-NB at different time points after 2 hours of ischemia followed by reperfusion. At 6 hours after reperfusion pairwise comparisons between hindlimb IRI+PBS vs. hindlimb IRI+Psel.B (0.5 mg) (p<0.05) and hindlimb IRI+PBS vs. hindlimb IRI+Psel.NB (0.5 mg) (p<0.05). Differences between the other comparisons were not significant. At 24 hours after reperfusion pairwise comparisons hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05),hindlimb IRI+Psel.NB (0.25 mg) vs. hindlimb IRI+Psel.NB (0.5 mg) (p<0.05) and hindlimb IRI+Psel.B (0.25 mg) vs. hindlimb IRI+Psel.NB (0.25 mg) (p<0.05). Differences between the other comparisons were not significant.

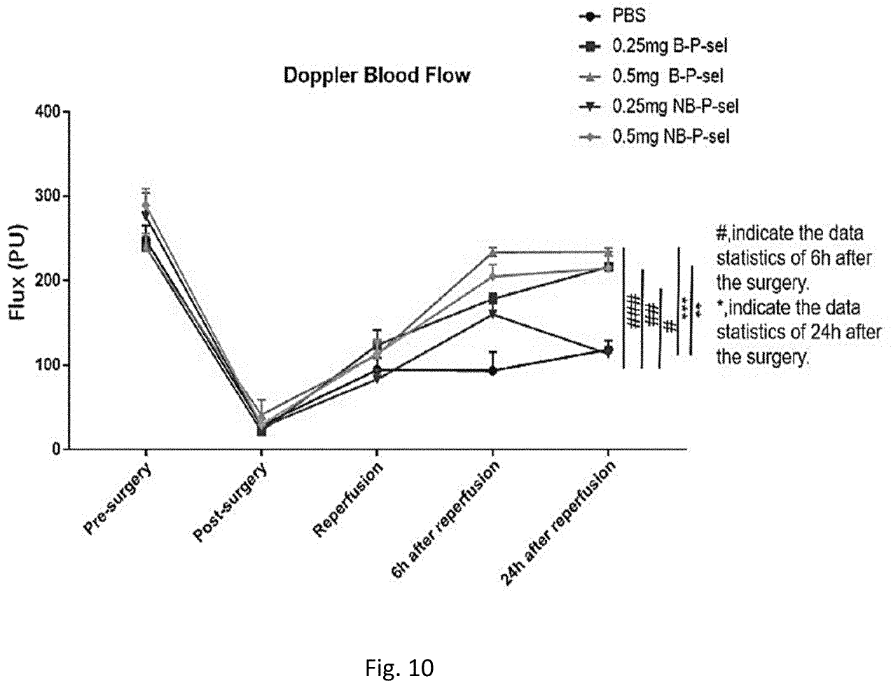

FIG. 10 shows a time course of recovery of blood flow in hindlimb IRI after treatment with different doses of Psel-B and Psel-NB at different time point after 2 hours of ischemia and followed by reperfusion. At 6h after reperfusion pairwise comparisons between hindlimb IRI+PBS vs. hindlimb IRI+Psel.B (0.25 mg) (p<0.05), hindlimb IRI+PBS vs. hindlimb IRI+Psel.B (0.5 mg) (p<0.05) and hindlimb IRI+PBS vs. hindlimb IRI+Psel.NB (0.5 mg) (p<0.05). Differences between the other comparisons were not significant. At 24 hours after reperfusion pairwise comparisons hindlimb IRI+PBS vs. hindlimb IRI+Psel.B (0.25 mg) (p<0.05), hindlimb IRI+PBS vs. hindlimb IRI+Psel.B (0.5 mg) (p<0.05), hindlimb IRI+PBS vs. hindlimb IRI+Psel.NB (0.5 mg) (p<0.05),hindlimb IRI+Psel.NB (0.25 mg) vs. hindlimb IRI+Psel.NB (0.5 mg) (p<0.05) and hindlimb IRI+Psel.B (0.25 mg) vs. hindlimb IRI+Psel.NB (0.25 mg) (p<0.05). Differences between the other comparisons were not significant.

FIG. 11 shows the bleeding time measured in hindlimb IRI after treatment with different doses of Psel-B and Psel-NB following 2 hours of ischemia and 6 hours reperfusion. Pairwise comparisons hindlimb IRI+PBS vs. hindlimb IRI+Psel.B (0.5 mg) (p<0.05), hindlimb IRI+Psel.B (0.25 mg) vs. hindlimb IRI+Psel.B (0.5 mg) (p<0.05) and hindlimb IRI+Psel.B (0.5 mg) vs. hindlimb IRI+Psel.NB (0.5 mg) (p<0.05). Differences between the other comparisons were not significant.

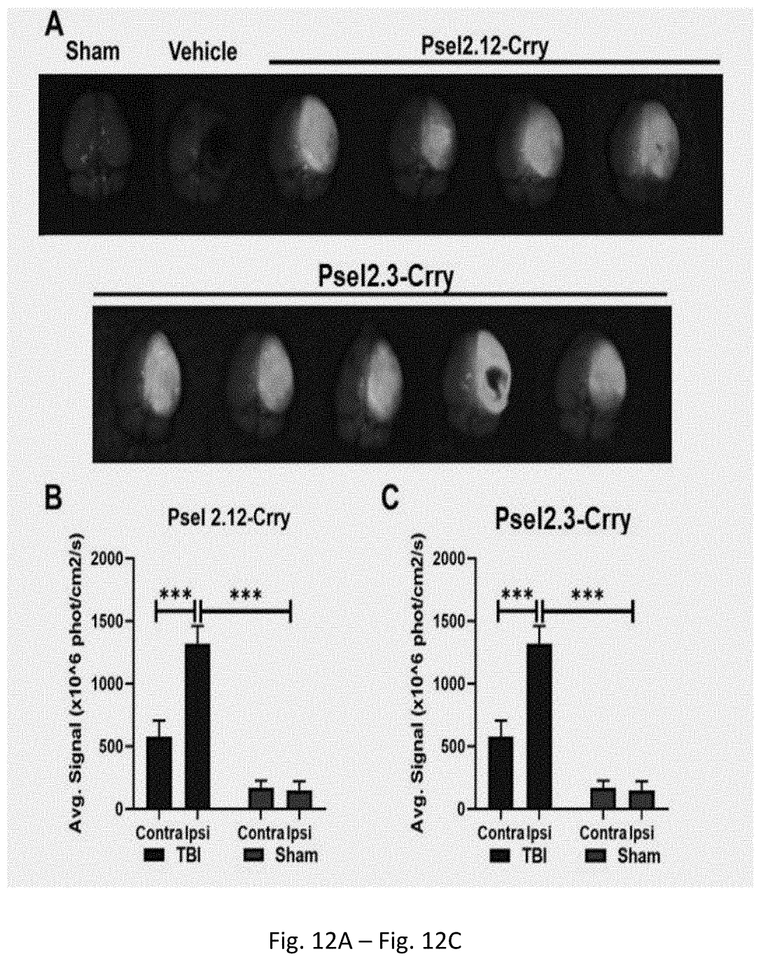

FIG. 12 shows in-vivo binding to brain after traumatic brain injury. Mice were subjected to traumatic brain injury (moderately severe injury) using the controlled cortical impact model involving the right hemisphere. At 2 hours after brain injury, either Psel2.12-Crry or Psel2.3-Crry that are fluorescently labeled were administered via tail-vein injections at 10 mg/kg dose. Brains were extracted at 24 hours and imaged ex-vivo to determine target localization. FIG. 12A depicts heatmaps of ex-vivo brains from different treatment group showing the signal of the construct in hot colors. Both Psel-Crry constructs targeted specifically to the right hemisphere (site of brain trauma) and minimal binding was observed in the contralateral hemisphere or in sham or vehicle animals. FIG. 12B and FIG. 12C are graphs which represent quantification of signal observed in FIG. 12A, and demonstrate significantly higher binding in the ipsilateral hemisphere (right) compared to left in animals subjected to brain trauma, and significantly higher binding in right hemisphere of brain trauma animals compared to sham. Similar pattern was observed for both inhibitors. N=4-5/group. Two-way ANOVA used for comparisons. ***P<0.001. Bars represent mean+/−SEM.

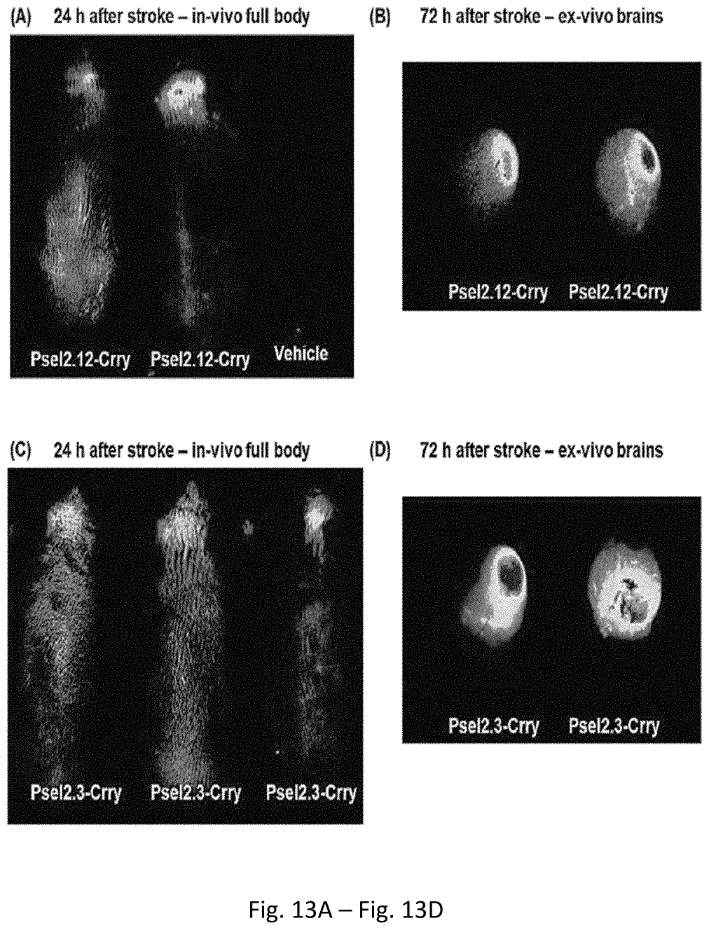

FIG. 13 shows in-vivo binding to brain after stroke. At 2 hours after brain injury, either Psel2.12-Crry or Psel2.3-Crry that are fluorescently labeled were administered via tail-vein injections at 10 mg/kg dose. Live animal in-vivo imaging was performed at 24 hours and brains were extracted and imaged ex-vivo to determine target localization at 72 hours. FIG. 13A and FIG. 13C depict heatmaps of animals from different treatment group showing the signal of the construct in hot colors. Both Psel-Crry constructs targeted specifically to the brain (site of stroke) and minimal binding was observed in the rest of the body or in vehicle-treated animals. FIG. 13B and FIG. 13D depict heatmaps of ex-vivo brains from different treatment group showing the signal of the construct in hot colors. Both Psel-Crry constructs targeted specifically to the right hemisphere (site of brain trauma) and minimal binding was observed in the contralateral hemisphere or in sham or vehicle animals.

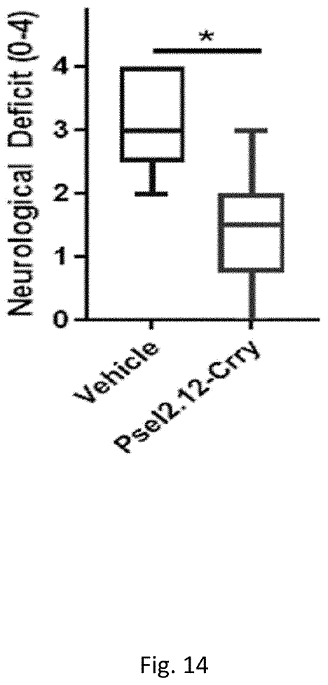

FIG. 14 shows acute neuroprotection by Psel 2.12 following stroke. Animals were assessed for neurological recovery at 24 hours after stroke using the neurological deficit score (0-4) with 4 being the worst score. The score is used to mimic the deficit scores used in human stroke. Animals treated with Psel2.12-Crry had significant reduction in neurological deficit scores compared to vehicle. Mann-Whiteny test used. N=6 (vehicle), 10 (Psel2.12-Crry). *P<0.05. Median and range are shown.

Other targeting vehicles for C inhibition have been investigated, most notably inhibitors that target the C activation product C3d via a CR2 targeting domain. However, there are several significant benefits to the proposed Psel targeted C inhibition approach: First, based on multiple studies investigating Psel blockade, it is expected that the targeting vehicle itself (for the blocking construct) will contribute to therapeutic activity by inhibiting immune cell extravasation. Since Psel can directly activate C, the targeting moiety may also possess additional C inhibitory activity. In addition, the anti-P-selectin domain has potential to modulate the coagulation cascade and platelet function. Second, unlike CR2-mediated targeting, it will not necessarily limit the expression of its ligand. Third, compared to CR2 targeting, the current strategy will likely be more specific for sites of stress/injury, since CR2 also binds other ligands, such as IFNalpha, CD23, DNA containing complexes, Epstein-Barr virus, as well as sites of spontaneous C activation such as kidney tubules. Fourth, although CR2-targeting limits the requirement for systemic inhibition, without being bound by theory, it was predicted that Psel targeting is even less immunosuppressive since CR2 can also bind pathogens marked for destruction by C3 opsonization, and thus inhibit their clearance. And fifth, the use of an Ab fragment provides a much more versatile targeting component. In addition to single chain constructs, there are possibilities for multiple engineered forms such as Fab or F(ab)2 fragments, whole mAbs with engineered Fc regions, multivalent constructs, etc.

### Example 3: P-Selectin-Crry for Complement Inhibition Following Germinal Matrix Hemorrhage

Germinal matrix hemorrhage (GMH) is a disease of infancy that affects neonates who are born premature or underweight. It occurs in a region in the brain near the ventricles called the “subventricular zone” that contains fragile vessels. It is also the site of progenitor cell proliferation; cells like neurons and oligodendrocytes that are essential for neurodevelopment.

Once the germinal matrix hemorrhage occurs, post-hemorrhagic hydrocephalus (PHH) becomes a significant risk. In the acute period, PHH can occur from physical obstruction of the ventricles by blood products. However, after blood products are cleared or broken down, an inflammatory response remains and creates damage to the walls of the ventricles as well as forces increased production of cerebrospinal fluid by the choroid plexus.

Chronically, there is still evidence of cyclic inflammation that results in scar formation, white matter loss, and loss of the ependymal lining within the ventricles. The result is chronic, irreversible hydrocephalus and pathologies that lead to cerebral palsy and severe neurodevelopmental delay.

Here, experiments were designed and conducted to examine the effect of the p-selectin targeted complement inhibitors described herein. The study consists of a novel mouse model that uses collagenase to allow breakdown of blood vessels in the subventricular zone (SVZ). This process mimics that of the GMH that occurs in neonates. The animals are injured at day 4 of life, and are followed until 14 days of life. Some cognitive testing is performed. Weights are measured during the study. At the end of the study, the animals are sacrificed and brains are extracted for histologic analysis.

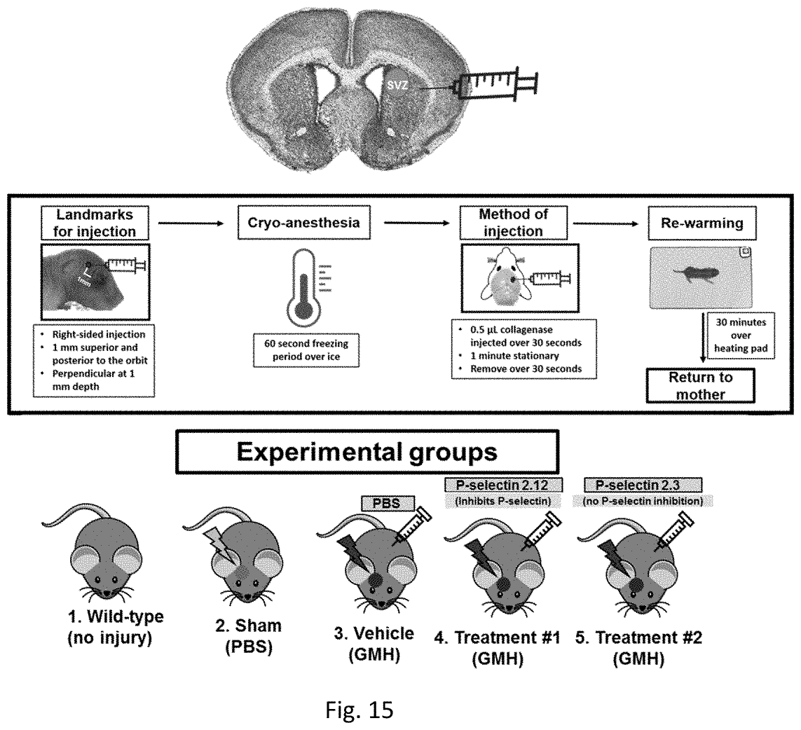

In this study (FIG. 15), 5 animal groups were used:

Wild-type: normal animal, no injury

Sham: The brain injury is caused by injection of PBS, not collagenase. This is to demonstrate that the study is caused purely by collagenase, not needle insertion.

Vehicle: Injury caused by collagenase. The animal is treated with PBS only, intraperitoneally.

Treatment #1: Injury caused by collagenase. The animal is treated with P-selectin 2.12 linked to Crry (C3 convertase inhibitor AND P-selectin inhibitor), intraperitoneally.

Treatment #2: Injury caused by collagenase. The animal is treated with P-selectin 2.3 linked to Crry (C3 convertase inhibitor WITHOUT P-selectin inhibition), intraperitoneally.

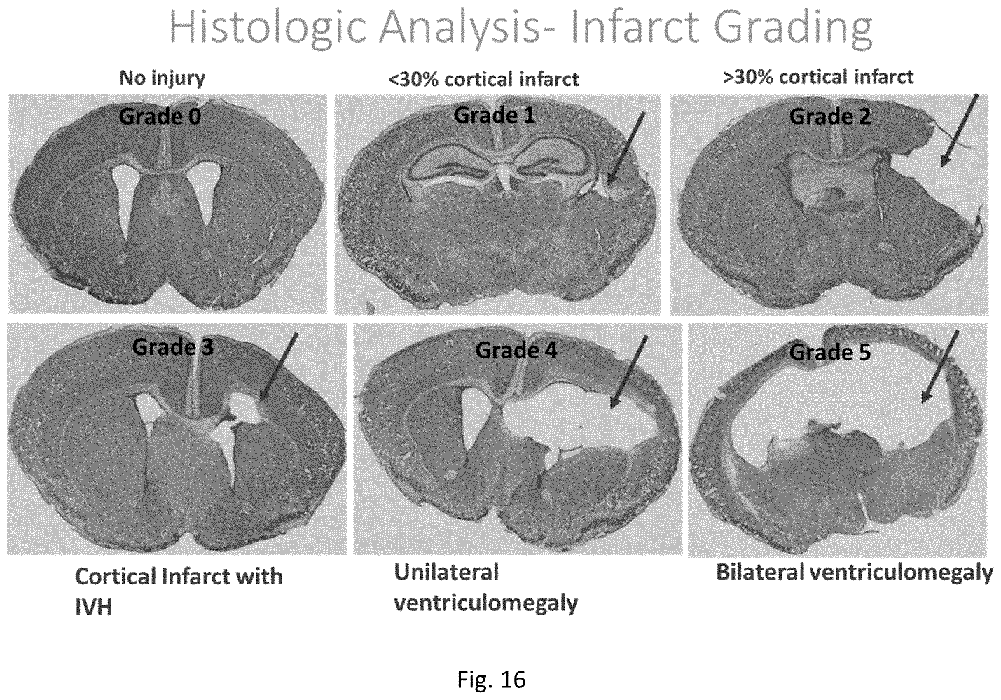

FIG. 16 depicts the grading system for analysis of GMH brains. Grade 0 shows no injury (normal brain). Grades 1 and 2 are infarcts without ventricular involvement. Grade 3 involves the ventricle but does not enlarge any ventricles. Grade 4 involves the ventricle AND enlarges the ipsilateral ventricle. Grade 5 involves ventricles and enlarges BOTH ventricles, causing global ventriculomegaly. Grade 5 is clinical hydrocephalus.

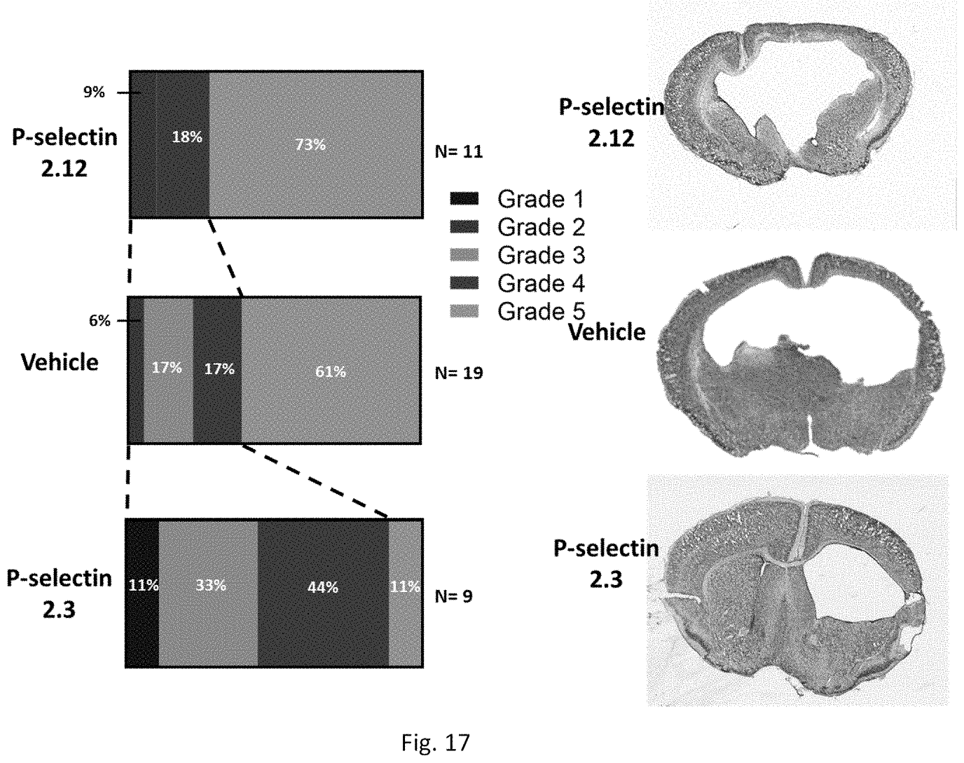

The rate of post-hemorrhagic hydrocephalus was examined in the treated animals, as assessed by Nissl histology (FIG. 17). FIG. 17 (left) demonstrates 61% hydrocephalus rate (grade 5) in vehicle animals; 73% hydrocephalus rate (grade 5) in P-selectin 2.12 animals (has p-selectin inhibition); and 11% hydrocephalus rate (grade 5) in P-2.3 animals (No p-selectin inhibition). Of note, PBS-injected brains had a 0% rate of hydrocephalus. On the right side of FIG. 17, examples are shown of representative brains from each group.

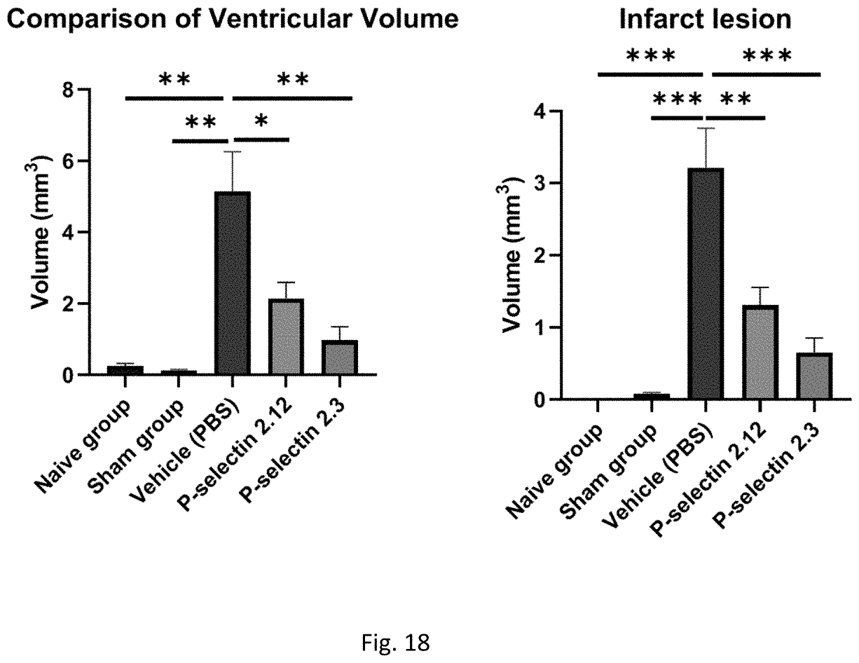

FIG. 18 depicts the Nissl histology results for ventricular volume and infarct lesion. As seen in FIG. 18 (left), ventricular volumes appear to be much lower in P-sel 2.3 compared to P-sel 2.12 and Vehicle. Interestingly, although the rate of hydrocephalus was higher in P-sel 2.12 compared to vehicle, the overall ventricular volume was still lower. In addition, the infarct lesion for P-selectin 2.12 was also lower compared to vehicle (FIG. 18 (right)). Overall, P-selectin 2.3 demonstrates significant improvement of hydrocephalus, ventricle volume, and infarct lesion compared to the vehicle and P-sel 2.12.

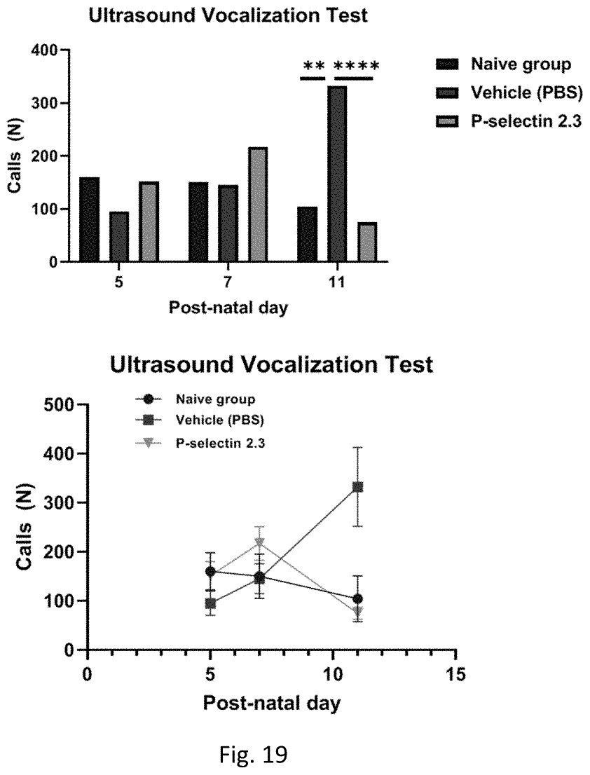

Further experiments were performed using the ultrasound vocalization test (USV). USV is a tool that measures number of “calls” made by infant pups to their mothers when in distress. These calls are typically within the ultrasonic range and thus require a specialized listening device to measure calls. The USV testing was performed on days 5, 7, and 11 of life. Here, a significant increase in distress calls was observed for vehicle animals at day 11 of life (injury was at P4). Animals treated with P-selectin 2.3 showed no difference in calls to naive animals (FIG. 19).

Further experiments are performed using flow cytometry testing to evaluate inflammatory cell recruitment and extravasation following P-selectin inhibition. P-selectin is a surface protein that works as a cell-adhesion molecule as well as propagates the complement system. Evaluation of cell infiltration is important in the context of continued inflammation. Experiments are also performed to evaluate platelet aggregation. Additionally, IF/IHC staining is performed to evaluate p-selectin expression following GMH injury (IHC with p-selectin); surrounding inflammation (C3/Iba-1, GFAP), and neuroprotection (NeuN, Olig-2, Synapses).

### Example 4

Evaluation of the Binding Affinity of Mouse Antigen P-Selectin to B.PselscFv-Crry and NB. PselscFv-Crry.

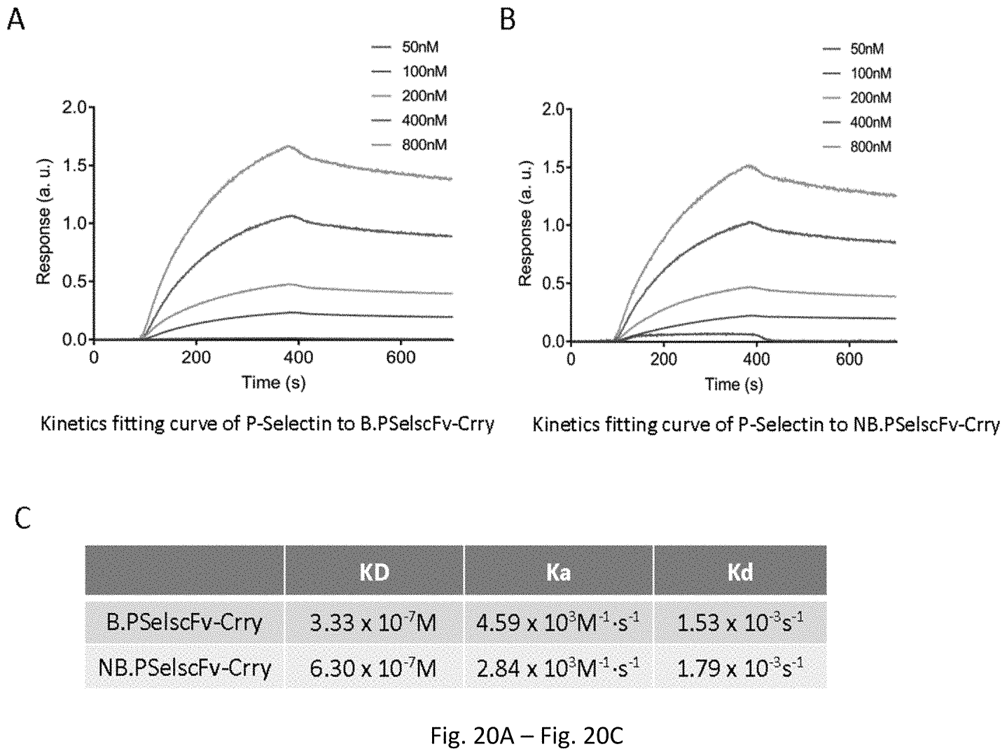

To measure binding affinity, surface plasmon resonance was used with mouse P-selectin as the immobilized ligand and B.PselscFv-Crry (FIG. 20A) and NB. PselscFv-Crry (FIG. 20B) as the analytes. Various concentrations of P-Selectin dissolved in water were manually printed onto the bare gold-coated (thickness 47 nm) PlexArray Nanocapture Sensor Chip (Plexera Bioscience, Seattle, Wash., US) at 40% humidity. Each concentration was printed in replicate, and each spot contained 0.2 μL of sample solution. The chip was incubated in 80% humidity at 4° C. for overnight, and rinsed with 10×PBST for 10 min, 1×PBST for 10 min, and deionized water twice for 10 min. The chip was then blocked with 5% (w/v) non-fat milk in water overnight, and washed with 10×PBST for 10 min, 1×PBST for 10 min, and deionized water twice for 10 min before being dried under a stream of nitrogen prior to use. SPRi measurements were performed with PlexAray HT (Plexera Bioscience, Seattle, Wash., US). Collimated light (660 nm) passes through the coupling prism, reflects off the SPR-active gold surface, and is received by the CCD camera. Buffers and samples were injected by a non-pulsatile piston pump into the 30 μL flowcell that was mounted on the coupling prim. Each measurement cycle contained four steps: washing with PBST running buffer at a constant rate of 2 μL/s to obtain a stable baseline, sample injection at 5 μL/s for binding, surface washing with PBST at 2 μL/s for 300 s, and regeneration with 0.5% (v/v) H3PO4 at 2 μL/s for 300 s. All the measurements were performed at 25° C. The signal changes after binding and washing (in AU) are recorded as the assay value. Selected protein-grafted regions in the SPR images were analyzed, and the average reflectivity variations of the chosen areas were plotted as a function of time. Real-time binding signals were recorded and analyzed by Data Analysis Module (DAM, Plexera Bioscience, Seattle, Wash., US). Kinetic analysis was performed using BIAevaluation 4.1 software (Biacore, Inc.). The calculated binding constants (KD), association rate constants (Ka) and dissociation rate constants (Kd) are shown in FIG. 20C.

B.PSelscFv-Crry and NB.PSelscFv-Crry Inhibit Complement Activation in a Dose-Dependent Manner and Bind Human P-Selectin Antigen.

To evaluate the ability of B.PselscFv-Crry and NB. PselscFv-Crry to inhibit complement activation, a zymosan A bead assay was performed. Briefly, flow cytometic analysis of C3 deposition on zymosan A particles (SigmaAldrich) was performed and the extent of complement inhibition was normalized by the subtraction of post-IRI complement desposition levels from their baseline levels. Both B.PSelscFv-Crry and NB.PSelscFv-Crry constructs inhibit complement activation in a dose-dependent manner (FIG. 21A). Further, to evaluate the binding of B.PselscFv-Crry and NB. PselscFv-Crry to human P-selectin, an ELISA was performed. Briefly, non-blocking and blocking fusion proteins (NB.PselscFv-Crry and B.PselscFv-Crry, respectively) were plated at varying doses in addition to varying doses of C2-Crry as a negative control. As expected, C2-Crry did not exhibit binding, but both of B.PselscFv-Crry and NB. PselscFv-Crry exhibited a dose-dependent increase in binding to human P-selectin.

B.PSelscFv-Crry (B.PSel) and NB.PSelscFv-Crry (NB.PSel) Reduces Injury Associated with Ischemia-Reperfusion In Vivo.

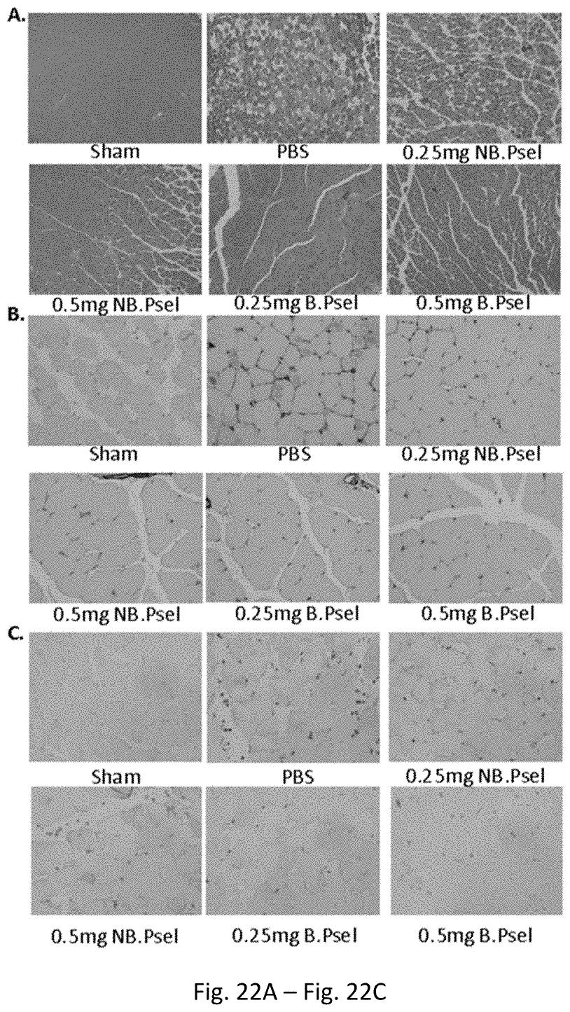

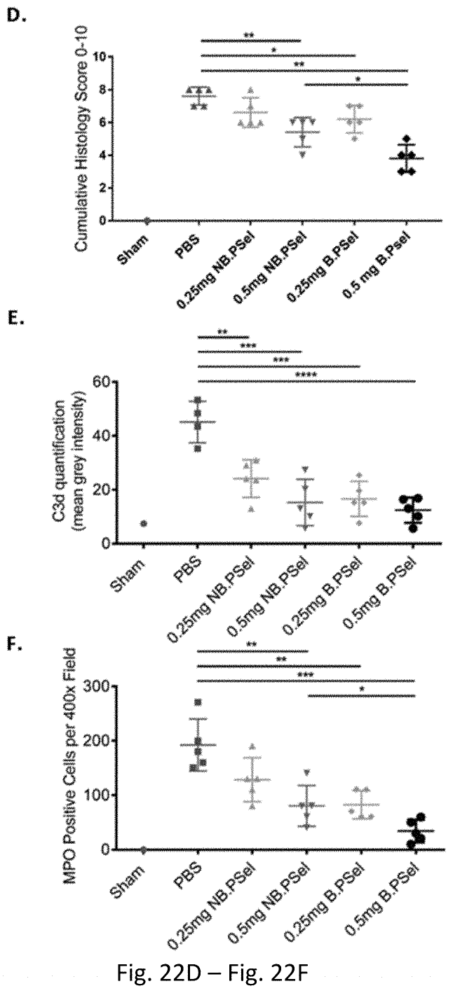

To test the effects of B.PselscFv-Crry (B.PSel) and NB. PselscFv-Crry (NB.PSel) on ischemia reperfusion injury (IRI), a murine hindlimb IRI model was used with two separate doses of 0.25 mg and 0.5 mg each. Histopathological scoring was performed on H&E stained tissue sections (FIG. 22A) and a significant, dose-dependent reduction in injury was observed for all except the lowest dose (0.25 mg) of NB.PSel (FIG. 22D). Histopathology sections were also stained for C3d (FIG. 22B) and significant reduction in complement deposition was observed for all treatment groups (FIG. 22E). Finally, myeloperoxidase (MPO) immunohistochemical (IHC) staining was performed on tissue sections (FIG. 22C) and a significant reduction in MPO was observed for all except the lowest dose (0.25 mg) of NB.PSel (FIG. 22F).

B.PSel Reduces P-Selectin Recruitment and Increases Bleeding Time Following Hindlimb IRI

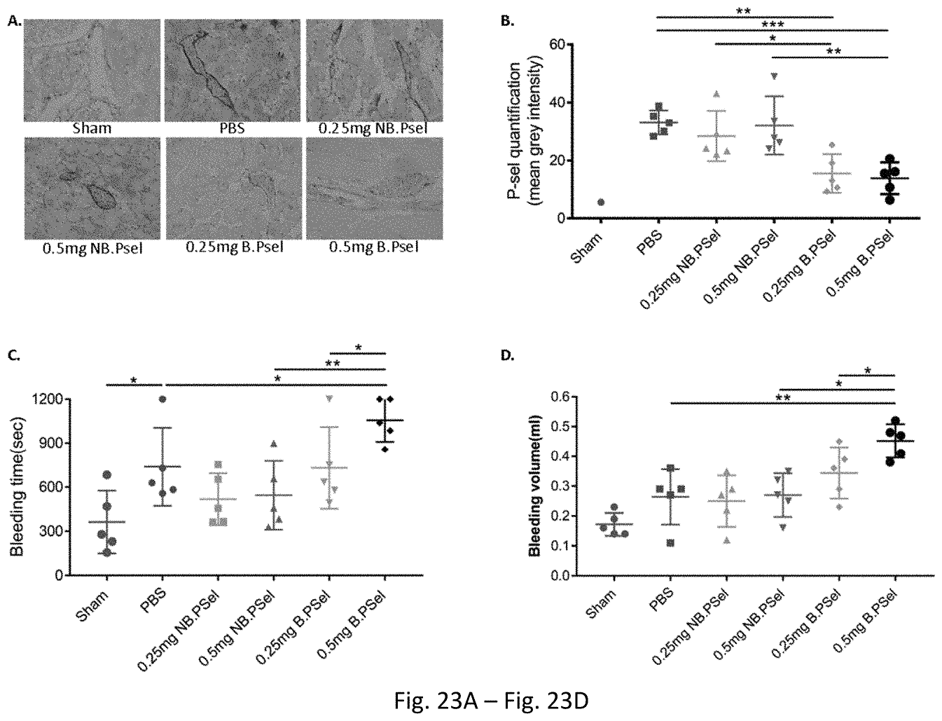

To assess the effects of B.PSel and NB.PSel on P-selectin recruitment, a murine hindlimb IRI model was used with two separate doses of 0.25 mg and 0.5 mg each. Histpathology sections were stained for P-selectin by IHC (FIG. 23A). As expected, NB.PSel did not block P-selectin recruitment, while B.PSel resulted in a significant reduction in P-selectin recruitment at both doses (FIG. 23B). Further, the effect of B.PSel and NB.PSel on bleeding was measured 2 hours after injection following hindlimb IRI. Animals were anesthetized with a mixture of ketamine and xylazine (100 and 10 mg/kg, respectively) after body weight (mg) was obtained. Animals were placed in prone position and a distal 5 mm segment of the tail was amputated with a scalpel. The tail was immediately immersed in a 50 mL conical tube containing pre-warmed isotonic saline at 37° C. The position of the tail was vertical with the tip positioned about 2 cm below the body horizon. Each animal was monitored for 20 minutes even if bleeding ceased in order to detect any re-bleeding. Bleeding time was determined using a stop clock. If bleeding on/off cycles occurred, the sum of bleeding times within the 20-minute period was used. The experiment was terminated at the end of 20 minutes to avoid lethality during the experiment as required by the local animal ethics committee. Body weight, including the tail tip, was measured again, and the volume of blood loss during the experimental period was estimated from the reduction in body weight. At the end of experiment, animals were euthanized by anesthetic overdose. Only the highest does of B.PSel resulted in a significant increase in bleeding time (FIG. 23C) and bleeding volume (FIG. 23D) relative to the PBS control.

B.PSel and NB.PSel Improve Perfusion in Hindlimb IRI Model In Vivo.

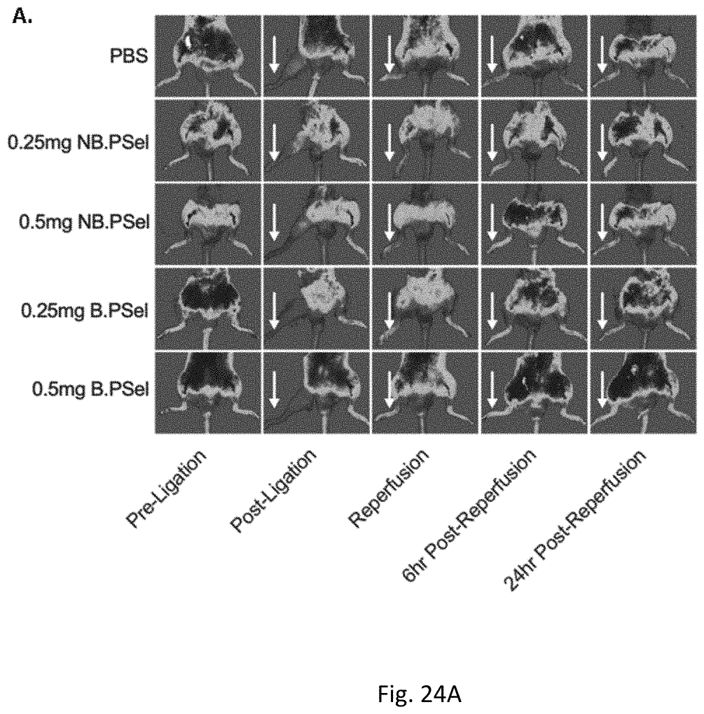

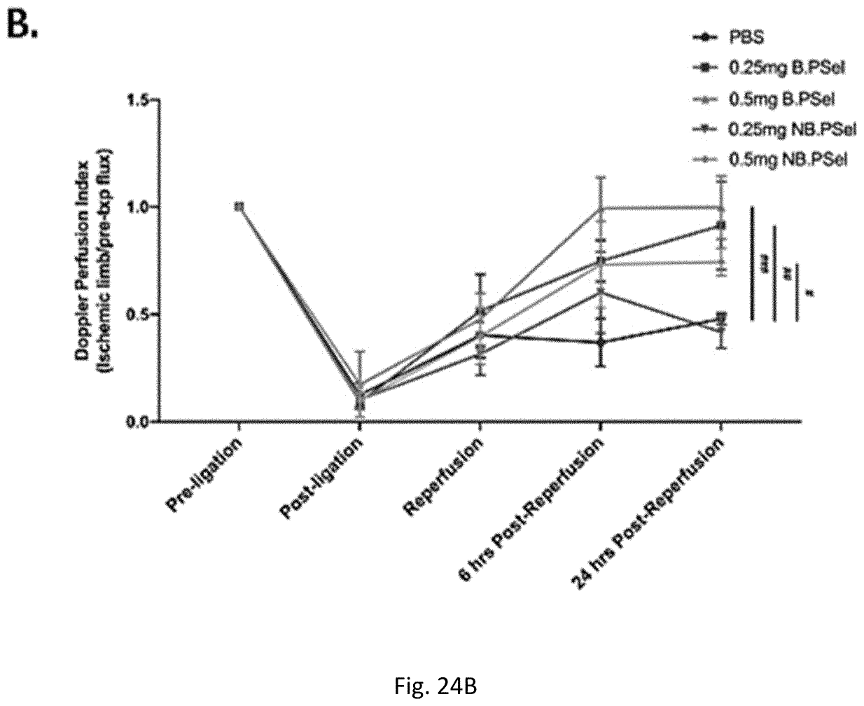

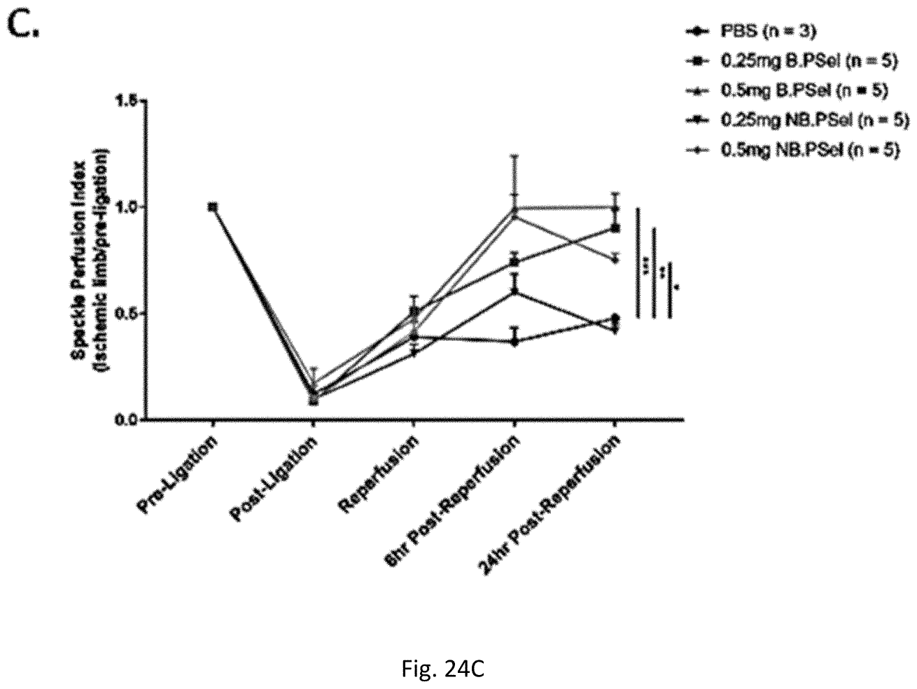

To determine the effects of B.PSel and NB.PSel on reperfusion, a murine hindlimb ligation IRI model was used with two separate doses of 0.25 mg and 0.5 mg each. FIG. 24A depicts representative laser speckle doppler images for each group, with the ligated limb identified with an arrow. Quantification of doppler measurements normalized to pre-ligation revealed significant improvements 6 hours post-reperfusion for all treatment groups using conventional doppler measurements ((p<0.05; FIG. 24B) and with administration of 0.5 mg of NB.PSel (p<0.05), 0.25 mg B.PSel (p<0.01), and 0.5 mg B.PSel (p<0.01) using laser speckle doppler measurements (FIG. 24C). At 24 hours post-reperfusion, a significant improvement in perfusion was seen following administration with 0.5 mg NB.PSel (#p<0.05), 0.25 mg B.PSel (##p<0.01), and 0.5 mg B.PSel (###p<0.01) using conventional doppler while treatments of 0.25 mg B.PSel (**p<0.01) and 0.5 mg B.PSel (***p<0.01) resulted in significant improvement in perfusion with laser speckle doppler.

Serum Circulatory Half-Life of B.PSel and NB.PSel.

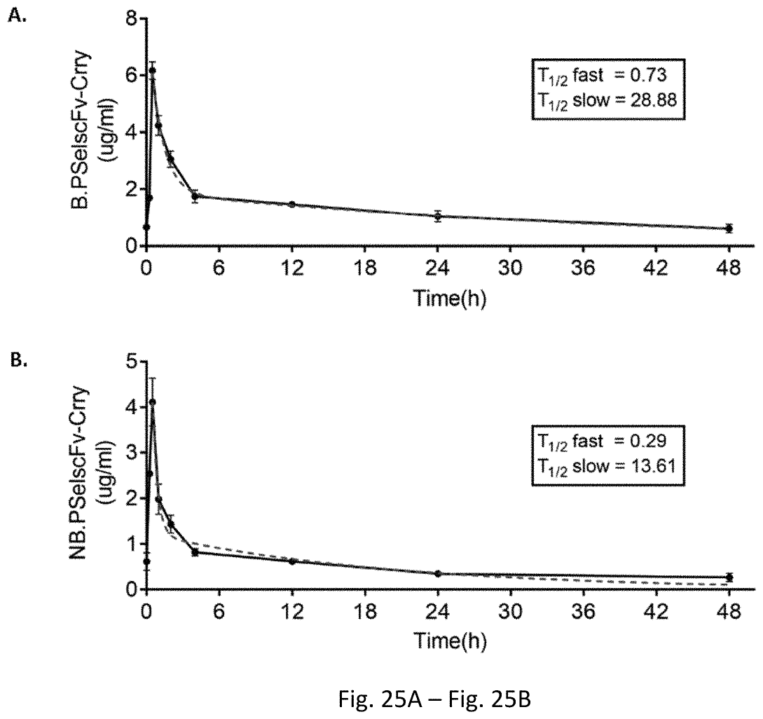

To evaluate the serum circulatory half-life of B.PSel and NB.PSel, a dose of 0.5 mg was administered i.v., and blood samples collected at indicated times for analysis of protein construct levels by anti-P-selectin ELISA. Fitting of the exponential decay curves and calculation of the pharmacokinetic parameters revealed a fast half-life of 0.73 h and slow half-life of 28.88 h for B.PSel (FIG. 25A) and a fast half-life of 0.29 h and slow half-life of 13.61 h for NB.PSel (FIG. 25B).

B.PSel and NB.PSel Specifically Traffic to Site of IRI In Vivo.

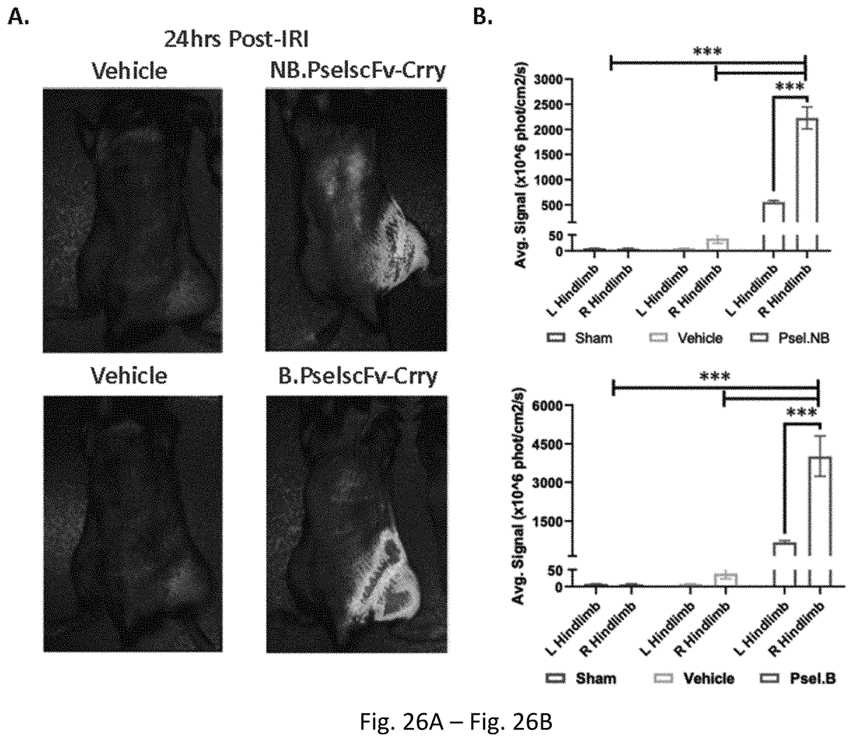

To determine where B.PSel and NB.PSel traffic in vivo, a biodistribution analysis was performed in a mouse hindlimb IRI model. Following 2 hours of ischemia time, 0.1 ml vehicle control, 0.25 mg fluorescently labeled NB.PSel, or 0.25 mg fluorescently labeled B.PSel were injected i.p. into each mouse. At 24 hrs post-reperfusion mice were imaged using a near-infrared Maestro in-vivo fluorescent imaging system. Animal procedures were approved by the Medical University of South Carolina Animal Care and Use Committee (IACUC). FIG. 26A depicts representative fluorescence images of vehicle and B.PSel or NB.PSel-treated mice 24 hours post-IRI. Quantification of the biodistribution to unaffected or ligated hindlimb shows that both NB.PSel (FIG. 26B, top) and B.PSel (FIG. 26B, bottom) preferentially traffic to the injured hindlimb.

B.PSel Reduces Graft Injury, MPO, and C3d Deposition Following Syngeneic Hindlimb Transplantation at 6 and 24 Hours Post-Transplantation of Vascularized Composite Isografts (VCI).

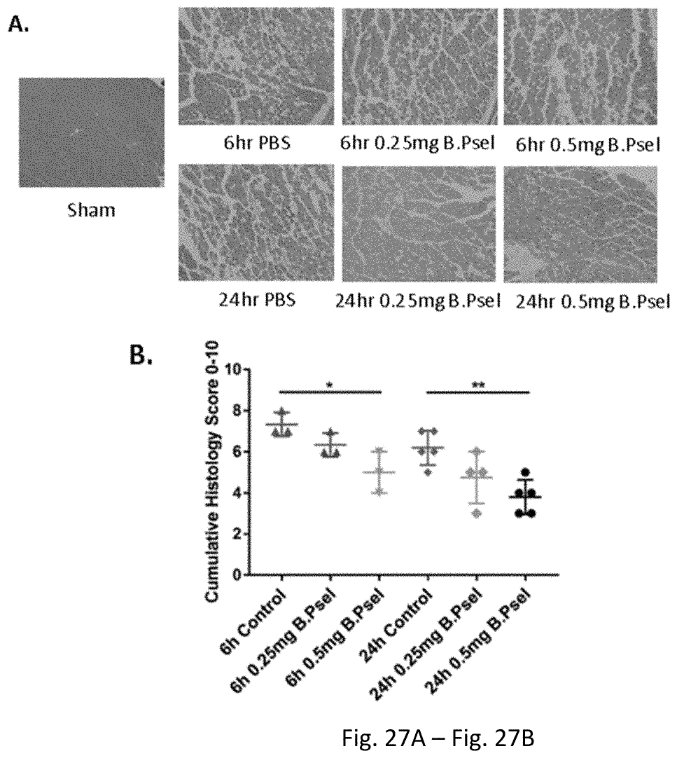

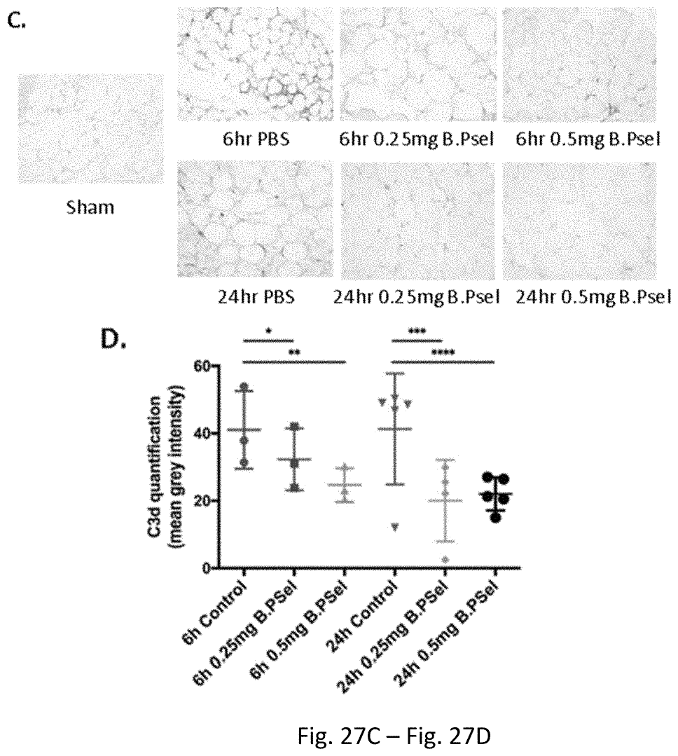

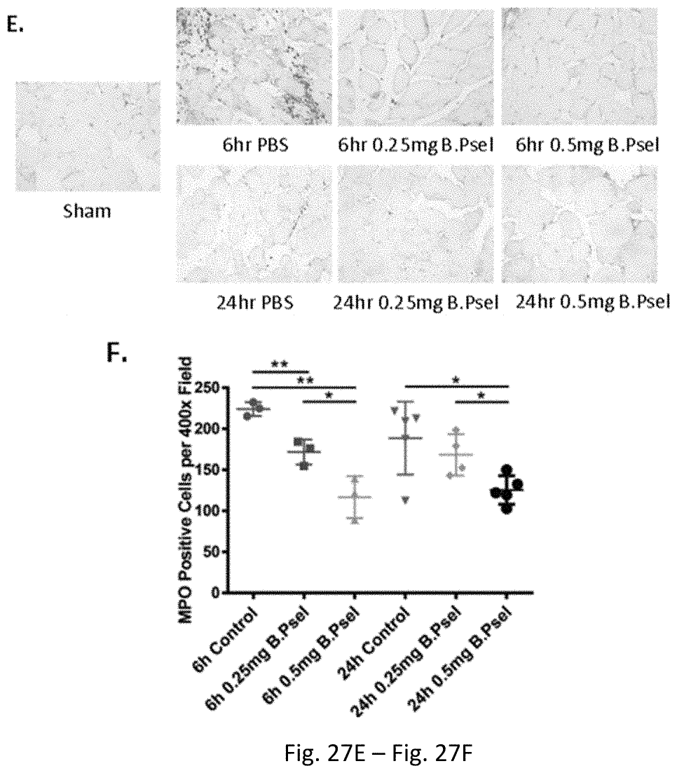

To test the effects of B.PSel on tissue transplantation from a genetically identical donor, tissue sections from the thigh muscle of each mouse hindlimb were taken at either 6 hours or 24 hours post-transplant in a VCI mouse model. H&E staining of tissue sections shows necrosis and neutrophilic infiltrates of controls, which is largely absent in B.PSel treated animals (FIG. 27A). Quantification using a histology score reveals significant reduction in injury for B.PSel relative to controls 6 hours and 24 hours post-transplant (FIG. 27B). Quantification of C3d IHC stained tissue sections (representative images depicted in FIG. 27C) revealed a dose dependent decrease in C3d deposition 6 hours and 24 hours post-transplantation, reaching statistical significance for 0.5 mg B.PSel (FIG. 27D). Finally, quantification of MPO IHC stained tissue sections (representative images depicted in FIG. 27E) shows a dose dependent and significant decrease in MPO for all treatments except 0.25 mg B.PSel 24 hours post-transplantation.

B.PSel Improves Hindlimb Perfusion 24 Hours Post-Transplantation of VCI.

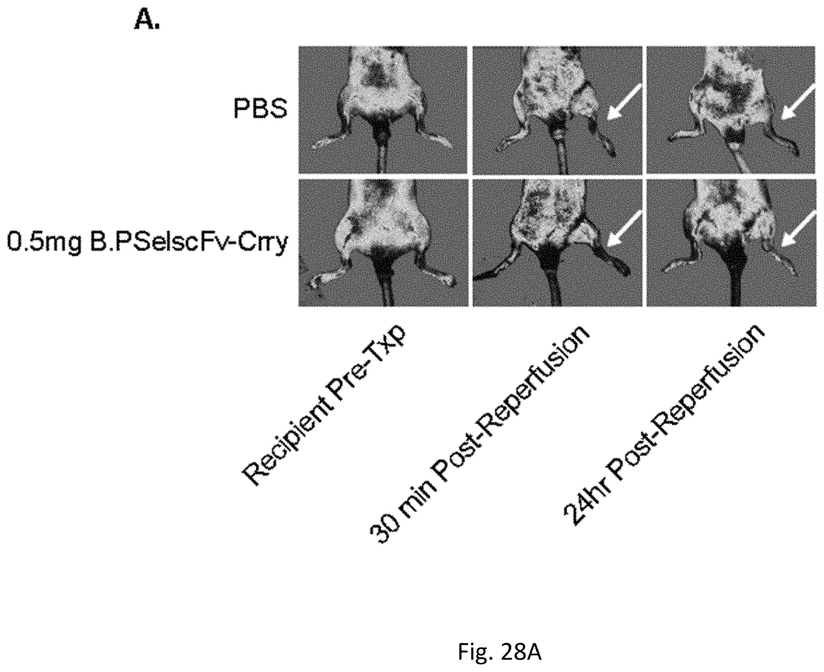

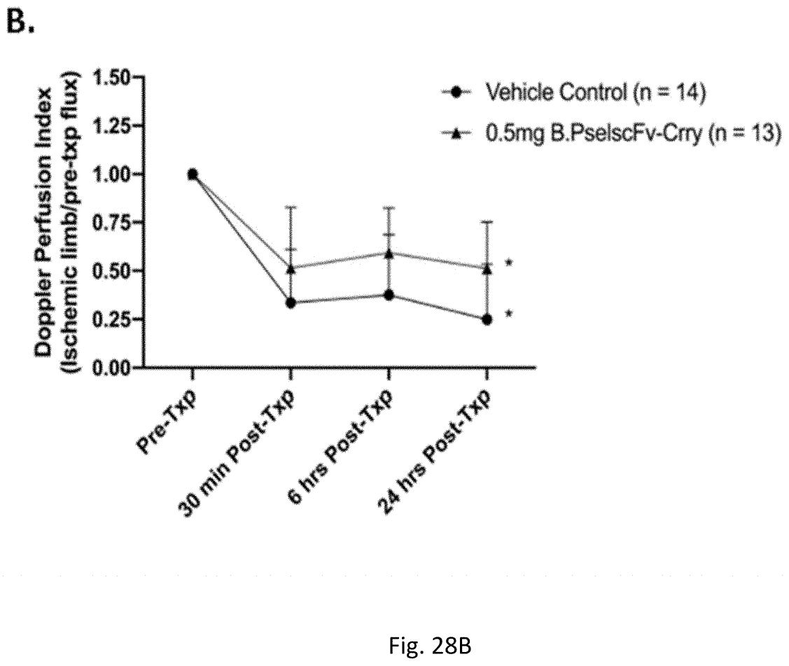

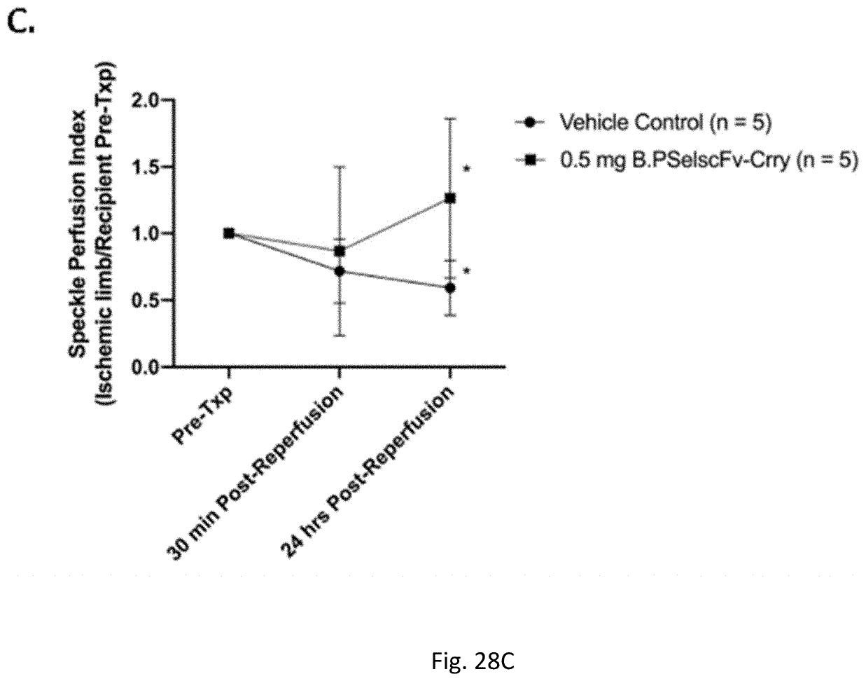

To evaluate the effect of B.PSel on perfusion following tissue transplantation from a genetically identical donor, conventional and laser speckle doppler imaging was performed in a hindlimb VCI model. FIG. 28A depicts representative laser speckle doppler images pre-transplantation, and 30 min and 24 hours post-reperfusion with vehicle or 0.5 mg B.PSel. Quantification by conventional doppler (FIG. 28B) and laser speckle doppler (FIG. 28C) revealed a significant improvement in perfusion 24 hours post-transplantation.

B.PSel Improves Perfusion in Vascularized Composite Allografts (VCA) Hindlimb Transplantation.

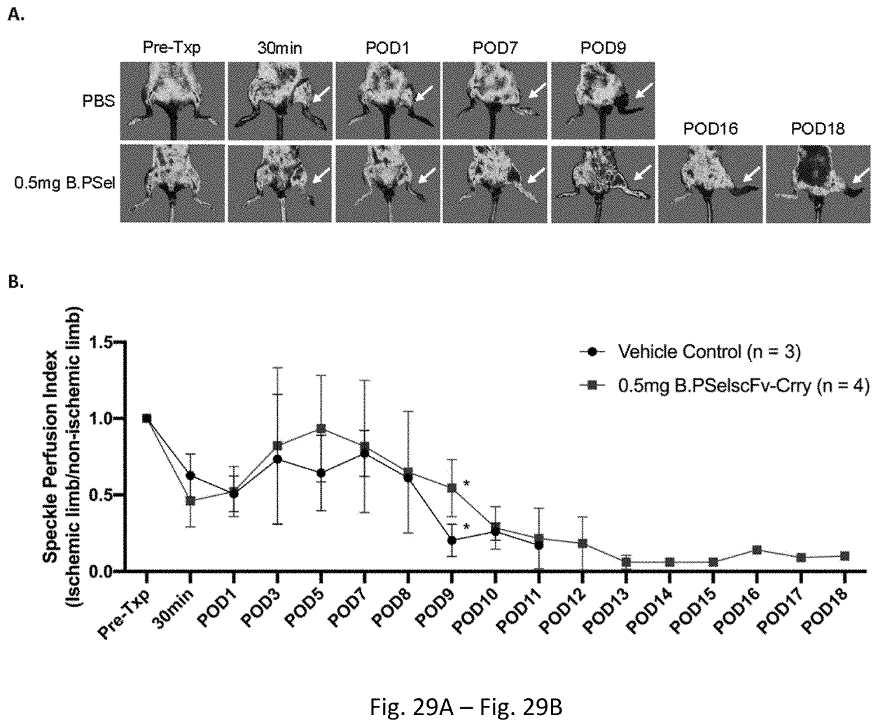

To evaluate the effect of B.PSel on perfusion following tissue transplantation from a genetically distinct donor of the same species, laser speckle doppler imaging was performed in a hindlimb VCA model. FIG. 29A depicts representative laser speckle doppler images pre-transplantation, 30 min post-reperfusion, and at post-operative days 1, 7, 9, 16 and 18. While a single postoperative dose of 0.5 mg B.PSel significantly improved hindlimb perfusion measured at day 9 post-transplantation (*p<0.05), no other time points resulted in a significant improvement in perfusion (FIG. 29B).

B.PSel Improves Survival of Hindlimb VCA.

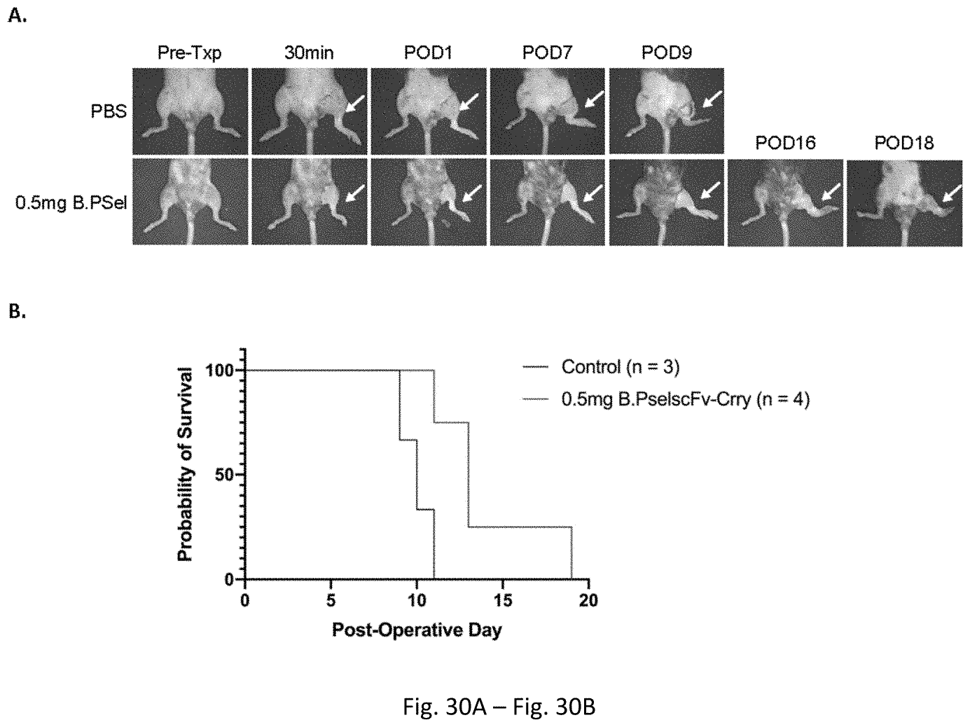

To evaluate the effect of B.PSel on allograft survival following tissue transplantation from a genetically distinct donor of the same species, a hindlimb VCA model was used and classified as days until Banff clinical grade 4 rejection was reached. FIG. 30A depicts representative gross images of hindlimb VCA transplanted mice are shown at pre-transplantation of the recipient (Pre-Txp), 30 minutes post-reperfusion, and at postoperative days 1, 7, 9, 16, and 18. FIG. 30B demonstrates a significant improvement in hindlimb allograft survival (p<0.05) following a single postoperative dose of 0.5 mg B.PSel.

### Sequences:

TABLE 2
CDR Sequences

| | SEQ ID | Amino Acid | SEQ ID | |
|---|---|---|---|---|
| Name | NO: | Sequence | NO: | Nucleotide Sequence |
| 2.3scFv HC | 13 | GYTFTTYG | 14 | GGCTACACCTTCACC |
| CDR1 | | | | ACCTACGGC |
| 2.3scFv HC | 15 | INTSSGVP | 16 | ATCAACACCAGCTCC |
| CDR2 | | | | GGCGTGCCT |
| 2.3scFv HC | 17 | ARGGGYYGAYYF | 18 | GCTCGTGGCGGAGGC |
| CDR3 | | YY | | TACTACGGAGCCTAC |
| | | | | TACTTCTACTAT |
| 2.3scFv LC | 19 | DNINSY | 20 | GATAACATCAACAGC |
| CDR1 | | | | TAT |
| 2.3scFv LC | 21 | NAK | 22 | AACGCCAAG |
| CDR2 | | | | |
| 2.3scFv LC | 23 | QHHYGPPPT | 24 | CAGCACCACTACGGC |
| CDR3 | | | | CCTCCCCCCACA |
| 2.7scFv HC | 13 | GYTFTTYG | 25 | GGGTATACCTTCACA |
| CDR1 | | | | ACCTATGGA |
| 2.7scFv HC | 15 | INTSSGVP | 26 | ATAAACACCTCCTCT |
| CDR2 | | | | GGAGTGCCA |
| 2.7scFv HC | 17 | ARGGGYYGAYYF | 27 | GCAAGAGGGGGGGG |
| CDR3 | | YY | | CTACTATGGTGCCTA |
| | | | | CTACTTTTACTAC |
| 2.7scFv LC | 28 | KSVSTSGYSY | 29 | AAAAGTGTCAGTACA |
| CDR1 | | | | TCTGGCTATAGTTAT |
| 2.7scFv LC | 30 | LVS | 31 | CTTGTATCC |
| CDR2 | | | | |
| 2.7scFv LC | 32 | QHIRELTRSEGGPS | 33 | CAGCACATTAGGGAG |
| CDR3 | | WK | | CTTACACGTTCGGAG |
| | | | | GGGGGACCAAGCTGG |
| | | | | AAA |
| 2.12scFv HC | 34 | GFTFSDYY | 35 | GGCTTCACCTTCTCCG |
| CDR1 | | | | ACTACTAC |
| 2.12scFv HC | 36 | INYDGSSA | 37 | ATCAATTACGACGGC |
| CDR2 | | | | AGCAGCGCC |
| 2.12scFv HC | 38 | ARGDWFVY | 39 | GCCAGGGGCGACTGG |
| CDR3 | | | | TTCGTCTAC |
| 2.12scFv LC | 40 | QDINSY | 41 | CAGGACATCAACAGC |
| CDR1 | | | | TAC |
| 2.12scFv LC | 42 | RAN | 43 | AGGGCCAAC |
| CDR2 | | | | |
| 2.12scFv LC | 44 | LQYAEFPFT | 45 | CTCCAGTATGCCGAG |
| CDR3 | | | | TTCCCCTTCACC |

SEQ ID NO:1—pCold2.3scFv (non-blocking), also referred to herein as Psel.NB or NB.PSelscFv, CDR sequences:

SEQ ID NO:14, SEQ ID NO:16, SEQ ID NO:18, SEQ ID NO:20, SEQ ID NO:22 and SEQ ID NO:24

SEQ ID NO:2—pCold2.3scFv (non-blocking), CDR sequences:

SEQ ID NO:13, SEQ ID NO:15, SEQ ID NO:17, SEQ ID NO:19, SEQ ID NO:21 and SEQ ID NO:23

SEQ ID NO:3—pEE12.4/anti-pselectin 2.3scFv-Crry (non-blocking), CDR sequences:

SEQ ID NO:14, SEQ ID NO:16, SEQ ID NO:18, SEQ ID NO:20, SEQ ID NO:22 and SEQ ID NO:24

SEQ ID NO:4—pEE12.4/anti-pselectin 2.3scFv-Crry (non-blocking), CDR sequences:

SEQ ID NO:13, SEQ ID NO:15, SEQ ID NO:17, SEQ ID NO:19, SEQ ID NO:21 and SEQ ID NO:23

SEQ ID NO:5—Pselectin 2.7scFv (non-blocking), CDR sequences:

SEQ ID NO:25, SEQ ID NO:26, SEQ ID NO:27, SEQ ID NO:29, SEQ ID NO:31 and SEQ ID NO:33

SEQ ID NO:6—Pselectin 2.7scFv (non-blocking), CDR sequences:

SEQ ID NO:13, SEQ ID NO:15, SEQ ID NO:17, SEQ ID NO:28, SEQ ID NO:30 and SEQ ID NO:32

SEQ ID NO:7—PEE12.4/Pselectin 2.7scFv-Crry (non-blocking), CDR sequences:

SEQ ID NO:25, SEQ ID NO:26, SEQ ID NO:27, SEQ ID NO:29, SEQ ID NO:31 and SEQ ID NO:33

SEQ ID NO:8—PEE12.4/Pselectin 2.7scFv-Crry (non-blocking), CDR sequences:

SEQ ID NO:13, SEQ ID NO:15, SEQ ID NO:17, SEQ ID NO:28, SEQ ID NO:30 and SEQ ID NO:32

SEQ ID NO:9—pCold2.12scFv (blocking) also referred to herein as Psel.B or B.PSelscFv, CDR sequences:

SEQ ID NO:35, SEQ ID NO:37, SEQ ID NO:39, SEQ ID NO:41, SEQ ID NO:43 and SEQ ID NO:45

SEQ ID NO:10—pCold2.12scFv (blocking), CDR sequences in bold

SEQ ID NO:34, SEQ ID NO:36, SEQ ID NO:38, SEQ ID NO:40, SEQ ID NO:42, SEQ ID NO:44

SEQ ID NO:11—pEE12.4/anti-pselectin scFv2.12-Crry (blocking), CDR sequences:

SEQ ID NO:35, SEQ ID NO:37, SEQ ID NO:39, SEQ ID NO:41, SEQ ID NO:43 and SEQ ID NO:45

SEQ ID NO:12—pEE12.4/anti-pselectin scFv2.12-Crry (blocking), CDR sequences:

SEQ ID NO:34, SEQ ID NO:36, SEQ ID NO:38, SEQ ID NO:40, SEQ ID NO:42, SEQ ID NO:44

SEQ ID NO:46—Pselectin2.12scFv-CR1(1-10), comprising CDRs:

SEQ ID NO:35, SEQ ID NO:37, SEQ ID NO:39, SEQ ID NO:41, SEQ ID NO:43 and SEQ ID NO:45

SEQ ID NO:47—Pselectin2.12scFv-CR1(1-10), comprising CDRs:

SEQ ID NO:34, SEQ ID NO:36, SEQ ID NO:38, SEQ ID NO:40, SEQ ID NO:42 and SEQ ID NO:44

SEQ ID NO:48—Pselectin2.12scFv-CR1(1-17), comprising CDRs:

SEQ ID NO:35, SEQ ID NO:37, SEQ ID NO:39, SEQ ID NO:41, SEQ ID NO:43 and SEQ ID NO:45

SEQ ID NO:49—Pselectin2.12scFv-CR1(1-17) Protein seq (SEQ ID NO:49) comprising CDRs:

SEQ ID NO:34, SEQ ID NO:36, SEQ ID NO:38, SEQ ID NO:40, SEQ ID NO:42 and SEQ ID NO:44

SEQ ID NO:50—Pselectin2.3scFv-CR1(1-10), comprising CDRs:

SEQ ID NO:14, SEQ ID NO:16, SEQ ID NO:18, SEQ ID NO:20, SEQ ID NO:22 and SEQ ID NO:24

SEQ ID NO:51—Pselectin2.3scFv-CR1(1-10), comprising CDRs:

SEQ ID NO:13, SEQ ID NO:15, SEQ ID NO:17, SEQ ID NO:19, SEQ ID NO:21 and SEQ ID NO:23

SEQ ID NO:52—Pselectin2.3scFv-CR1(1-17), comprising CDRs:

SEQ ID NO:14, SEQ ID NO:16, SEQ ID NO:18, SEQ ID NO:20, SEQ ID NO:22 and SEQ ID NO:24

SEQ ID NO:53—Pselectin2.3scFv-CR1(1-17), comprising CDRs:

SEQ ID NO:13, SEQ ID NO:15, SEQ ID NO:17, SEQ ID NO:19, SEQ ID NO:21 and SEQ ID NO:23

What is claimed is:

1. An antibody or fragment thereof comprising a p-selectin binding domain that specifically binds to p-selectin.

2. The antibody or fragment thereof of claim 1, wherein the antibody is selected from the group consisting of a non-blocking anti-p-selectin binding antibody, and an anti-p-selectin blocking antibody.

3. The antibody or fragment thereof of claim 2, wherein the antibody comprises at least one selected from the group consisting of:
a) at least one CDR selected from the group consisting of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO: 23;
b) at least one CDR selected from the group consisting of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32;
c) at least one CDR selected from the group consisting of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44;
d) an amino acid sequence selected from the group consisting of SEQ ID NO:2, SEQ ID NO:6 and SEQ ID NO:10;
e) a sequence having at least 95% identity to an amino acid sequence selected from the group consisting of SEQ ID NO:2, SEQ ID NO:6 and SEQ ID NO:10; and
f) a fragment comprising at least 80% of the full-length sequence of an amino acid sequence selected from the group consisting of SEQ ID NO:2, SEQ ID NO:6 and SEQ ID NO:10.

4. A nucleic acid molecule encoding an antibody or fragment thereof of claim 1.

5. The nucleic acid molecule of claim 4, wherein the nucleic acid molecule comprises at least one nucleotide sequence encoding at least one CDR selected from the group consisting of:
a) at least one nucleotide sequence selected from the group consisting of a nucleotide sequence of SEQ ID NO:14 encoding a HC CDR1, a nucleotide sequence of SEQ ID NO:16 encoding a HC CDR2, a nucleotide sequence of SEQ ID NO:18 encoding a HC CDR3, a nucleotide sequence of SEQ ID NO:20 encoding a LC CDR1, a nucleotide sequence of SEQ ID NO:22 encoding a LC CDR2, and a nucleotide sequence of SEQ ID NO:24 encoding a LC CDR3;
b) at least one nucleotide sequence selected from the group consisting of a nucleotide sequence of SEQ ID NO:25 encoding a HC CDR1, a nucleotide sequence of SEQ ID NO:26 encoding a HC CDR2, a nucleotide sequence of SEQ ID NO:27 encoding a HC CDR3, a nucleotide sequence of SEQ ID NO:29 encoding a LC CDR1, a nucleotide sequence of SEQ ID NO:31 encoding a LC CDR2, and a nucleotide sequence of SEQ ID NO:33 encoding a LC CDR3;
c) at least one nucleotide sequence selected from the group consisting of a nucleotide sequence of SEQ ID NO:35 encoding a HC CDR1, a nucleotide sequence of SEQ ID NO:37 encoding a HC CDR2, a nucleotide sequence of SEQ ID NO:39 encoding a HC CDR3, a nucleotide sequence of SEQ ID NO:41 encoding a LC CDR1, a nucleotide sequence of SEQ ID NO:43 encoding a LC CDR2, and a nucleotide sequence of SEQ ID NO:45 encoding a LC CDR3;
d) at least one nucleotide sequence selected from the group consisting of SEQ ID NO:1, SEQ ID NO:5 and SEQ ID NO:9;
e) a sequence having at least 95% identity to at least one nucleotide sequence selected from the group consisting of SEQ ID NO:1, SEQ ID NO:5 and SEQ ID NO:9; and
f) a fragment comprising at least 80% of the full-length sequence of at least one nucleotide sequence selected from the group consisting of SEQ ID NO:1, SEQ ID NO:5 and SEQ ID NO:9.

6. A fusion molecule comprising a p-selectin binding domain comprising a molecule that specifically binds to p-selectin fused to a cargo domain comprising a complement inhibitor.

7. The fusion molecule of claim 6, wherein the molecule that specifically binds to p-selectin is selected from the group consisting of a non-blocking anti-p-selectin binding antibody, and an anti-p-selectin blocking antibody.

8. The fusion molecule of claim 6, wherein the molecule that specifically binds to p-selectin comprises at least one selected from the group consisting of:
a) at least one CDR selected from the group consisting of a heavy chain (HC) CDR1 sequence of SEQ ID NO:13, a HC CDR2 sequence of SEQ ID NO:15, a HC CDR3 sequence of SEQ ID NO:17, a light chain (LC) CDR1 sequence of SEQ ID NO:19, a LC CDR2 sequence of SEQ ID NO:21, and a LC CDR3 sequence of SEQ ID NO:23;
b) at least one CDR selected from the group consisting of a HC CDR1 sequence of SEQ ID NO:13, a HC CDR2 sequence of SEQ ID NO:15, a HC CDR3 sequence of SEQ ID NO:17, a LC CDR1 sequence of SEQ ID NO:28, a LC CDR2 sequence of SEQ ID NO:30, and a LC CDR3 sequence of SEQ ID NO:32;
c) at least one CDR selected from the group consisting of a HC CDR1 sequence of SEQ ID NO:34, a HC CDR2 sequence of SEQ ID NO:36, a HC CDR3 sequence of SEQ ID NO:38, a LC CDR1 sequence of SEQ ID NO:40, a LC CDR2 sequence of SEQ ID NO:42, and a LC CDR3 sequence of SEQ ID NO:44;
d) a sequence selected from the group consisting of SEQ ID NO:2, SEQ ID NO:6, and SEQ ID NO:10;
e) a sequence having at least 95% identity to a sequence selected from the group consisting of SEQ ID NO:2, SEQ ID NO:6, and SEQ ID NO:10; and
f) a fragment comprising at least 80% of a full-length sequence selected from the group consisting of SEQ ID NO:2, SEQ ID NO:6, and SEQ ID NO:10.

9. The fusion molecule of claim 6, wherein the complement inhibitory domain inhibits at least one classical complement pathway, alternative complement pathway or lectin pathway protein selected from the group consisting of C1, manna binding lectin protease, C3 convertase, C5 convertase, and the membrane attack complex.

10. The fusion molecule of claim 6, wherein the complement inhibitor is selected from a protein, a peptide, a small molecule, a nucleic acid molecule, an antibody and an antibody fragment.

11. The fusion molecule of claim 6, wherein the complement inhibitory domain comprises at least one selected from the group consisting of Factor H (FH), Decay Accelerating Factor (DAF or CD55), Membrane Cofactor Protein (MCP or CD46), Protectin (CD59), Crry (murine equivalent of MCP), Mannose-binding lectin-associated protein of 44 kDa (MAp44), Complement C3b/C4b Receptor 1 (CR1 or CD35), Complement Regulator of the Immunoglobulin Superfamily (CRIg), C4-Binding Protein (C4 bp), OMS721, Eculizumab, Ravulizumab, Coversin, CCX168, IFX 1, CCX168, AMY-101, APL-2, ACH 4471, LPN023, Cemdisiran, C1INH, LFG-316, and plasma serine proteinase inhibitor serpin or a fragment thereof.

12. The fusion molecule of claim 6, wherein the complement inhibitory domain comprises CR1.

13. The fusion molecule of claim 6, wherein the fusion molecule comprises an amino acid sequence selected from the group consisting of
a) a sequence selected from the group consisting of SEQ ID NO:47, SEQ ID NO:49, SEQ ID NO:51, and SEQ ID NO:53;
b) a sequence having at least 95% identity to a sequence selected from the group consisting of SEQ ID NO:47, SEQ ID NO:49, SEQ ID NO:51, and SEQ ID NO:53; and
c) a fragment comprising at least 80% of a full-length sequence selected from the group consisting of SEQ ID NO:47, SEQ ID NO:49, SEQ ID NO:51, and SEQ ID NO:53.

14. A nucleic acid molecule encoding a fusion molecule of claim 6.

15. The nucleic acid molecule of claim 14, comprising a nucleotide sequence selected from the group consisting of:
a) at least one nucleotide sequence selected from the group consisting of a nucleotide sequence of SEQ ID NO:14 encoding a HC CDR1, a nucleotide sequence of SEQ ID NO:16 encoding a HC CDR2, a nucleotide sequence of SEQ ID NO:18 encoding a HC CDR3, a nucleotide sequence of SEQ ID NO:20 encoding a LC CDR1, a nucleotide sequence of SEQ ID NO:22 encoding a LC CDR2, and a nucleotide sequence of SEQ ID NO:24 encoding a LC CDR3;
b) at least one nucleotide sequence selected from the group consisting of a nucleotide sequence of SEQ ID NO:25 encoding a HC CDR1, a nucleotide sequence of SEQ ID NO:26 encoding a HC CDR2, a nucleotide sequence of SEQ ID NO:27 encoding a HC CDR3, a nucleotide sequence of SEQ ID NO:29 encoding a LC CDR1, a nucleotide sequence of SEQ ID NO:31 encoding a LC CDR2, and a nucleotide sequence of SEQ ID NO:33 encoding a LC CDR3;
c) at least one nucleotide sequence selected from the group consisting of a nucleotide sequence of SEQ ID NO:35 encoding a HC CDR1, a nucleotide sequence of SEQ ID NO:37 encoding a HC CDR2, a nucleotide sequence of SEQ ID NO:39 encoding a HC CDR3, a nucleotide sequence of SEQ ID NO:41 encoding a LC CDR1, a nucleotide sequence of SEQ ID NO:43 encoding a LC CDR2, and a nucleotide sequence of SEQ ID NO:45 encoding a LC CDR3;
d) a nucleotide sequence selected from the group consisting of SEQ ID NO:1, SEQ ID NO:5, SEQ ID NO:9, SEQ ID NO:46, SEQ ID NO:48, SEQ ID NO: 50 and SEQ ID NO: 52;
e) a sequence having at least 95% identity to a nucleotide sequence selected from the group consisting of SEQ ID NO:1, SEQ ID NO:5, SEQ ID NO:9, SEQ ID NO:46, SEQ ID NO:48, SEQ ID NO: 50 and SEQ ID NO:52; and
f) a fragment comprising at least 60% of the full length sequence of a nucleotide sequence selected from the group consisting of SEQ ID NO:1, SEQ ID NO:5, SEQ ID NO:9, SEQ ID NO:46, SEQ ID NO:48, SEQ ID NO: 50 and SEQ ID NO:52.

16. A composition comprising an antibody, or fragment thereof of claim 1, a nucleic acid molecule encoding the same, a fusion molecule comprising a p-selectin binding domain comprising a molecule that specifically binds to p-selectin fused to a cargo domain comprising a complement inhibitor, or a nucleic acid molecule encoding a fusion molecule comprising a p-selectin binding domain comprising a molecule that specifically binds to p-selectin fused to a cargo domain comprising a complement inhibitor.

17. The composition of claim 16, further comprising a pharmaceutically acceptable excipient.

18. A method for treating a disease or disorder associated with at least one of p-selectin activity and complement signaling in a subject comprising administering to the subject a therapeutically effective amount of a composition of claim 16.

19. The method of claim 18, wherein the disease or disorder is selected from the group consisting of ischemia, reperfusion injury, traumatic brain injury, intracranial hemorrhage, including germinal matrix hemorrhage (GMH) and intraventricular hemorrhage (IVH), post-hemorrhagic hydrocephalus (PHH), coronary artery disease, acute myocardial infarction, stroke, and peripheral artery diseases, allergy, asthma, any autoimmune diseases, celiac disease, glomerulonephritis, hepatitis, inflammatory bowel disease, transplant rejection, coagulopathies, thrombotic disorders, CNS injury, diseases of the CNS and peripheral nervous system, neurodegenerative disorders, ocular disorders, including glaucoma and age-related macular degeneration, infectious disease and pathologies of infectious disease (including but not limited to viral and bacterial infections, systemic organ involvement), blood and clotting disorders and inflammatory diseases and disorders.
